(19) United States
(12) Patent Application Publication
(10) Pub. No.: US 2023/0301719 A1
(43) Pub. Date: Sep. 28, 2023

(54) SYSTEMS AND METHODS FOR PLANNING SCREW LENGTHS AND GUIDING SCREW TRAJECTORIES DURING SURGERY

(71) Applicant: Stephen B. Murphy, M.D., Winchester, MA (US)

(72) Inventor: Stephen B. Murphy, M.D., Winchester, MA (US)

(21) Appl. No.: 18/126,634

(22) Filed: Mar. 27, 2023

Related U.S. Application Data
(60) Provisional application No. 63/323,839, filed on Mar. 25, 2022.

(51) Int. Cl. A61B 34/10; A61B 34/20

(52) U.S. Cl. CPC A61B 34/10; A61B 34/20; A61B 2034/2055

(57) ABSTRACT

Systems and methods plan the trajectories and lengths of screws used to secure an acetabular cup component within the acetabulum of a patient's pelvis and guide a surgeon, during the surgical procedure, to install the screws of the planned lengths along the planned trajectories. The screw trajectories and lengths may be determined based on the placement of the acetabular shell within the acetabulum.

### CROSS-REFERENCE TO RELATED APPLICATIONS

The present application claims the benefit of U.S. Provisional Patent Application Ser. No. 63/323,839 filed Mar. 25, 2022, which application is hereby incorporated by reference in its entirety.

### BACKGROUND

### Background Information

Traditional surgical navigation can be broken down into the type of tracking technology used and the type of imaging used, if any. Currently, the most common tracking technologies used for surgical navigation are either infrared stereoscopic optical tracking or inertial tracking. Electromagnetic tracking can be used as well but much less frequently so now. Infrared stereoscopic optical tracking has the limitation that the camera needs its own line of site to the surgical field and it can only track specific objects that have reflective spheres that reflect infrared light or have active light emitting diodes (LEDs) that emit infrared light. Such tracking is incapable of seeing, recognizing, and spatially tracking objects.

With respect to imaging, the basic types of navigation are image-based and image-free. Image-based navigation typically involves using Computed Tomography (CT), Magnetic Resonance (MR) imaging, or 3D Ultrasound and may include the pre-operative or intra-operative development of three-dimensional (3D) models of a patient's anatomy. This computer model of the patient's anatomy is then matched to the actual patient's anatomy through a registration process during surgery after a tracker is affixed to the patient's anatomy. Similarly, navigation analogous to image-based navigation involves substituting 3D models from patient-specific imaging with predictive models of the patient, such as statistical shaped models. For example, a predicted 3D model may be generated for a patient—as opposed to an actual 3D model for the patient—based on 2D X-rays of the patient and information from a large data set of patient statistics and/or statistic shaped models.

For image-free registration, a tracker is similarly affixed to the patient's anatomy but the anatomy is not registered to a 3D model derived from imaging. For example, in the case of image-free navigation for hip arthroplasty, measuring prosthetic acetabular cup orientation and calculating leg length change using image-free navigation techniques involves affixing a tracker to the pelvis. Using one image-free method, the pelvis is then “squared-up”, and that position is set to be the starting functional coordinate system for the pelvis. Other instruments are navigated relative to that.

With a second, more typical image-free prosthetic cup and leg length navigation, a skeletal reference frame (tracker) is affixed to the pelvis and a coordinate system such as the Anterior Pelvic (AP) Plane coordinate system is defined relative to the tracker. The AP Plane coordinate system is defined using a digitizer and entering the two superior spine points and the pubic symphysis to instruct the system as to where the tracker is located in space relative to the digitized coordinate system.

For image-based registration, after a pelvic tracker is affixed to the pelvis, a digitizer is used to digitize various points on the pelvic bone surface to achieve spatial registration between the computer model of the patient's pelvis and patient's actual pelvis.

Similarly, the HipXpert® tool from Surgical Planning Associates, Inc. of Medford, MA can be used as a registration device, after a pelvic tracker is affixed, by digitizing the three divots on the tool after the tool is predictably docked to the patient's pelvis. In some embodiments, registration of the pelvis as determined by the HipXpert tool with an image tracker mounted on the tool may be transferred to a tracker randomly attached to the patient's pelvis, for example without having to digitize points on the tool. In other embodiments, which may be referred to as virtual registration, a patient's pelvis may be registered by randomly attaching a tracker to the patient's pelvis and digitizing the points at which the HipXpert tool, as planned, would dock to the patient's pelvis, but free from docking a physical HipXpert tool to the patient's pelvis. The HipXpert tool is described in U.S. Pat. No. 8,267,938 for a Method and Apparatus for Determining Acetabular Component Positioning, which is hereby incorporated by reference in its entirety.

Mechanical fixation devices are often used in orthopedic surgical procedures. For example, fixation devices are used to secure bone fragments following fractures. Fixation devices are also used to secure implants to bone structures, such as during joint reconstruction and other surgeries. Exemplary mechanical fixation devices include screws, pins, staples, nails, and wires.

In total hip arthroplasty, for example, an acetabular cup component may be implanted in the acetabulum of a patient's pelvis. The acetabular cup component may include a shell component that is secured to the acetabulum and a liner that is secured inside the shell. The shell, which may be metal, may be secured to the acetabulum using fixation devices, e.g., screws. During the procedure, the surgeon may choose the orientation of the shell within the acetabulum. Considering a vector extending along a center axis of a shell that extends perpendicular to an opening face of the shell, orientation may refer to the relation of this vector to the acetabulum. The surgeon may also choose a rotation of the shell within the acetabulum. Rotation may refer to the shell's rotation about the vector extending along the center axis of the, e.g., while the orientation of the shell within the acetabulum is held constant. The selected orientation and rotation of shell may determine the entry points into the pelvis for the screws used to secure the shell. That is, the shell can be both oriented and rotated within the acetabulum, thus changing the points at which the screws enter the pelvis. Choices to the shell's orientation during planning and surgery may be limited or constrained, e.g., by the shape or structure of the acetabulum. Rotation, on the other hand, can typically be freely selected during planning and surgery. That is, the shell may be widely or broadly rotated within the acetabulum. The surgeon can also choose the lengths of the screws to be used. The surgeon may choose an orientation for the shell within the acetabulum and screw lengths that are as long as possible to firmly attach the shell to the pelvis. The screws, however, should not be so long as to extend beyond the interior surface of the pelvis as doing so can damage patient tissue.

The thickness of the pelvis at the acetabulum, however, can vary, sometimes significantly. In addition, the surgeon typically may not have information on the thickness of the pelvis during surgery. For example, the surgeon may not have access to the area behind the acetabulum during surgery. Because of these conditions, it can be difficult for a surgeon to choose the optimal orientation and/or rotation of the shell and the lengths of the screws. A surgeon can attempt to determine the length of a screw by using a drill bit and figuring out how deep the drill bit had gone in before the drill bit went through the far cortex. Alternatively, the surgeon may use a depth gauge with a hook to physically measure how long a screw should be. Often, a surgeon will place the shell in a position, e.g., in terms of x,y,z coordinates, an orientation, and a rotation within the patient's acetabulum before considering screw lengths. This preplacement can force the surgeon into using screws that are shorter than they otherwise might be, for example if the shell were to be placed in a different position, orientation, and/or rotation. In some cases, a surgeon may choose screws whose lengths are shorter than might otherwise be possible to ensure that the screws do not extend beyond the pelvis and into the patient's tissue. Using screws that are too short, however, can result in the acetabular cup component failing post-surgery given the high loads imposed on the hip joint. A surgeon may also consider bone quality, bone strength and the intrinsic fit between the shell and the bone, when deciding on screw length. Some surgeons may decide whether to use screws at all depending upon the feel of the purchase between the shell and pelvis.

Accordingly, a need exists for systems and methods to assist surgeons in determining optimum placement of acetabular cup components, including shells, and optimum screw lengths.

### SUMMARY

Briefly, the present disclosure relates to systems and methods for utilizing mixed reality (MR) also referred to as Augmented Reality (AR) devices to perform registration and/or navigation during surgical procedures. In some embodiments, the MR device may include processors, memory, sensors, and one or more projection systems for displaying virtual images to the user of the MR device, among other elements. Exemplary sensors include photo/video cameras, depth cameras, light sensors, and microphones, among others. Exemplary images include holograms, e.g., objects made from light and sound.

As described, a patient-specific surgical plan may be developed in which the locations of surgical tools and/or implants are planned so as to achive one or more goals of the surgery. The planned locations may be determined relative to a coordinate system associated with a portion of the patient's anatomy, such as the patient's pelvis, femur, tibia, heart, lung, etc. The planned locations also may be translated to be relative to the coordinate system associated with a registration and tracking device that may be affixed to the patient or the planned locations may be originally determined relative to the coordinate system associated with the registration and tracking device. The systems and methods may generate virtual images, such as holograms, of the registration and tracking device, as custom configured for the patient, and of the surgical tools and/or implants at the planned locations. Virtual images of the patient's anatomy or portions thereof may also be generated. During surgery, with the patient in the operating room, patient registration is performed. In some embodiments, patient registration is performed using the registration and tracking device. For example, the hologram of the registration and tracking device may be presented and co-located, e.g., aligned, with the physical registration and tracking device affixed to the patient in the planned manner, for example manually by the surgeon, automatically by the systems and methods, and/or a combination of manual and automatic techniques. In other embodiments, patient registration may be performed based on object recognition by the systems and methods of a portion of the patient's anatomy, such as recognition of the patient's femoral condyles, the tibial plateau or the acetabulum as exposed during surgery, among other anatomical structures. Holograms of the surgical tools and/or implants in the planned locations may then be presented, and the physical surgical tools and/or implants may be manipulated, e.g., by the surgeon, to co-locate with the holograms, thereby achieving the one or more goals of the surgery. In some embodiments, the surgeon may manually mainpulate the hologram of the registration and tracking device and/or the physical registration and tracking device or the patient until the two are co-located. In other embodiments, the registration tracking device may include a recognizable image, for example one or more Quick Response (QR) or other codes. The systems and methods may detect that image, e.g., the one or more QR codes, and automatically co-locate and anchor the hologram of the registration and tracking device with the physical registration and tracking device. In some embodiments, the systems and methods may recognize the registration and tracking device as configured for the patient and docked to the patient's anatomy, some portion of the patient's anatomy, such as a bone surface visisble through an incision, and/or some combination of QR codes, registration and tracking device, and patient anatomy. The systems and methods may continuously detect the spatial position and orientation of the image, the registration and tracking device, and/or the patient anatomy during surgery in order to keep the hologram co-located with the physical registration and tracking device.

As noted, in some embodiments, the systems and methods may recognize one or more objects during surgery. For example, the system and methods may recognize some portion of the patient's specific bony anatomy for patient registration and/or to anchor or co-locate one or more virtual images, e.g., holograms. In some embodiments, registration of the patient may be transferred from the registration and tracking device to another device, e.g., a tracking or anchoring device, allowing removal of the registration and tracking device. As noted, the registration and tracking device may be docked to the patient's anatomy. The tracking or anchoring device may be an implant following implantation, such as a prosthetic cup component implanted in the patient's acetabulum.

Shape data for one or more objects, such of which may be patient-specific objects may be generated pre-operatively. Exemplary objects include anatomical structures, such as the patient's pelvis, acetabulum, femur, tibia, etc., and surgical tools or devices some of which may be customized for the patient, such as tools or devices adjusted based on the patient's anatomy and templates fabricated to interfit with the patient's anatomy. The shape data may be in the form of one or more two-dimensional (2D), three-dimensional (3D), or 2D-3D models of the patient-specific object. In some embodiments, the models may be surface models while in other embodiments the models may be solid models. One or more coordinate systems may be defined pre-operatively, for example during a planning phase, based on the patient-specific object. Exemplary coordinate systems include a pelvic coordinate system, a femoral coordinate system, and/or a tibial coordinate system. The coordinate systems may be defined automatically, e.g., by a planning tool, manually by a planning surgeon or surgeon's trained associate, or through a combination of automated and manual steps. In addition, the location of one or more prosthetic components, such as a cup component and/or a femoral stem component, may be planned relative to the one or more coordinate systems. The term location may refer to six parameters determining the position and orientation of an object in space.

During a planning phase, three-dimensional (3D) models of anatomical structures, such as the pelvis, and devices and tools, such as the HipXpert pelvic registration and tracking device may be generated and used to plan the surgery for a patient. For example, specific prosthetic components may be selected and their locations within the patient's body determined, e.g., to meet one or more goals of the surgery. 3D models of surgical tools, such as reamers and cup impactors, may be generated and their locations for preparing the bone and for implanting the selected components at the desired locations planned. The desired locations may be final locations, e.g., of a particular tool, or a sequence of locations, e.g., a tool path, from a starting point of a tool to its final location. At least some of the 3D models may be exported into a form that may be used by the head-mounted MR device to generate respective virtual images. During the surgical procedure, the surgeon may wear the MR device, which may be an MR head-mounted device (HMD). The MR device may be configured to include or have access to a navigation system. The navigation system may cooperate with the MR device to generate one or more virtual images, which may be projected onto one or both of the lenses of the MR device, to assist in the surgical procedure. The one or more virtual images may be in the form of holograms of objects, and the holograms may appear from the surgeon's perspective to be in the surgical scene. As used herein, a hologram may be a 2D or 3D image that may be formed of light. In some embodiments, the surgeon may operate user interface controls to manually resize and move the holograms so that they are co-located with corresponding physical objects in the surgical scene. Once co-located by the surgeon, the holograms may be anchored at those locations. The surgeon may then operate one or more physical tools until the physical tools are co-located with holograms of the respective tools. With the physical tools co-located with the holograms of the respective tools, anatomical structures may be prepared to receive the prosthetic components as planned, and the selected components may be implanted at the planned locations.

As noted, in some embodiments, a recognizable image, e.g., a QR code, may be affixed to the registration and tracking device in a predetermined location. The systems and methods may detect and recognize this image, e.g., the QR code. Based on the recognition of the QR code, the systems and methods may co-locate the hologram of the registration and tracking device to the physical registration and tracking device. Holograms of the surgical tools at the planned locations may then be presented. In some embodiments, the systems and methods may omit presenting a hologram of the registration and tracking device and instead, having recognized the QR code on the physical registration and tracking device, merely present the holograms of the surgical tools at the planned locations. In some embodiments, multiple QR codes may be used. For example, different QR codes may be placed on the faces of a cube mounted to the registration and tracking device. Each QR code may expose a spatial coordinate system aligned with the QR code, for example at the top left corner of the finder pattern. The MR device may detect the spatial coordinate system associated with one or more of these QR codes. The systems and methods may detect the QR code and/or the spatial coordinate system repeatedly during the surgery, e.g., at some frequency such as 60 Hz, and thus continuously keep the hologram co-located with the physical registration and tracking device. For example, the MR device may detect the spatial position and orientation of the image, e.g., QR code(s), the registration and tracking device, and/or the patient anatomy at least periodically over some duration of the surgery, such as 60 Hz or some other frequency, intermittently, continuously, and/or occasionally. The systems and methods may also use an inertial measurement unit (IMU) to keep the hologram co-located with the physical registration and tracking device, for example if line of sight to the registration and tracking device and/or the QR code is lost at any point during the surgery. In some embodiments, the systems or methods may issue one or more alerts and/or warnings if line of sight to the registration and tracking device and/or QR code has been lost for long enough to risk loss of accurate co-location so that re-anchoring is recommended, which may be a predetermined time. For example, presentation of the hologram of the registration and tracking device or any other objects or tools may be stopped or suspended until re-anchoring is performed.

At least a portion of the registration and tracking device including the one or more QR codes may be disposed outside of the patient's body. As a result, the registration and tracking device including the one or more QR codes may be readily detected by the MR device. Nonetheless, virtual images, e.g., holograms, anchored based on the detection of the registration and tracking device may be presented to appear as though they extend into or are entirely disposed inside the patient's body.

In some embodiments, data from the surgical scene as captured by one or more sensors of the MR device may be processed by the navigation system that utilizes the pre-operatively obtained and/or determined shape data for an object, such as a patient-specific object, to detect the object in the surgical scene. This may be referred to as an object recognition mode in which the systems and methods create shape data for an object, such as a patient-specific object, preoperatively and then use object recognition techniques to anchor a virtual image to the real object. It should be understood that only a portion of the actual object may be observable in the data captured by the MR device. Nonetheless, the navigation system may detect the object and determine its location. The navigation system may next register the object, e.g., relative to the one or more pre-operatively determined coordinate systems based on the detection of the object and its determined location. In addition to registering to a coordinate system, the system, once recognizing and co-locating an object, may display a virtual image of any other object or tool onto the surgical scene in the planned location relative to the recognized object. The navigation system may also track the object during the surgical procedure. In some embodiments, registration and tracking of the object may be transferred to a second object, such as a tracker placed on the patient, thereby allowing the surgeon to alter the object originally recognized without losing the tracking ability, as some procedures may include modifying the object.

The navigation system may generate one or more virtual images, e.g., holograms, which may be projected onto the lenses of the MR device, to assist in the surgical procedure. For example, while only a small portion of the patient's pelvis may be visible through the incision, a hologram of the entire pelvis may be rendered by the MR device and the hologram may be co-located with the patient's physical pelvis. In other embodiments, holograms of the entire femur and/or tibia may be rendered and co-located with the patient's femur or tibia, as examples. Additionally or alternatively, holograms of the one or more coordinate systems and/or guides for implanting one or more prosthetic components at the planned locations may be rendered by the MR device and appear as though they are in the surgical field in order to assist the surgeon in placing the prosthetic components. In some embodiments, the locations of the prosthetic components may be changed during the surgical procedure, and the guides presented to the surgeon by the MR device may be updated to conform to these changes. This may be referred to as a live holography mode in which the systems and methods incrementally or continuously in real time update the holograms to reflect the work performed by the surgical tools, whether directed by the surgeon or by a robot.

The following outline presents one or more embodiments of the present disclosure. It should be understood that different combinations of these features may be implemented in different embodiments of the present disclosure.
1. Image or object recognition for registration and tracking of a registration and tracking device, such as the HipXpert tool, on a patient specific basis. This also registers the pelvis. Image or object recognition may include at least periodically detecting and/or recognizing an image or object over some duration of time during the surgical procedure, such as intermittently, continuously, and/or occasionally over the duration of time.
   a. augmented reality display of a virtual pelvis superimposed on the patient's pelvis from the surgeon's real-time perspective.
   b. transferring the pelvic registration to another recognizable tracking object so that the registration and tracking tool, e.g., the HipXpert tool, can be removed from the surgical field.
2. Automated registration of the pelvis based on a view of the acetabulum.
3. Combined registration using 1 and 2 to improve the accuracy of registration. An error in registration can appear visibly as double vision. Improving the accuracy may reduce or eliminate such double vision.
4. Virtual registration of the pelvis by randomly attaching a tracker and digitizing the three points at which the legs of the HipXpert tool are planned to contact the pelvis.
5. Prepare the acetabulum for total hip arthroplasty (THR), for example by lining up a physical cup impactor with a hologram of the cup impactor, perform periacetabular osteotomy, biopsy a lesion, and/or perform other surgical procedure.
   a. Track one or more tools used during the procedure and update the 3D models and/or holograms of the pelvis, femur, etc. based on what has happened so far in real time.
   b. Compare three structures during surgery: the original anatomical structure, the anatomical structure as modified thus far in the surgery, and the final goal of how the surgeon wants the anatomical structure to be modified.
6. Embodiments of the present disclosure may transfer registration from tracking the shape of the end of a bone (patient-specific object recognition) to another object, such as a tracker, so that the surgeon can start to modify the bone surfaces without losing tracking ability.
7. Example of endoscopic applications. Using an endoscopic camera that has stereoscopic vision and/or a depth camera, e.g., Time of Flight (ToF) sensors, embodiments of the present disclosure can register an object using an automated object recognition as matched to a 3D model of the same object. Then, if the MR device worn by the surgeon or a stereoscopic tracking system separate from the MR device located in the operating room, such as an Infra Red (IR) tracking system, can see a part of the external portion of the endoscope, the relative location of the MR device to the endoscope's point of view would allow the present disclosure to project virtual 3D objects onto the actual objects from the surgeon's exact point of view. For example, this may be:
   a. An endoscopic camera identifies the 3D location of a human body part using stereoscopy and or a combination of sensors to achieve automated 3D (object recognition) surface registration.
   b. Then, the back end of the endoscopic camera which exits the person's body can be registered and tracked by the present disclosure including the MR device and/or the IR tracking system, among others.
   c. The MR device may then present virtual images, e.g., holograms, of anatomical structures or objects. This allows the surgeon to “see” through the body and “see” the structures or objects virtually through the skin or any other opaque object in between the surgeon and the object. Optimal locations of ligament placement may be calculated and presented, e.g., by the MR device, as can optimal tunnel locations for accessing the calculated ligament placement locations.

In some embodiments, the present disclosure relates to computer-based systems and methods for creating a preoperative plan of a surgical procedure and creating one or more holograms that can be presented, for example during the surgical procedure. The systems and methods include one or more of a surgical planning system, an Augmented Reality Head-Mounted Display (MR-HMD) configured as a surgical guidance system, and one or more registration and tracking devices. The surgical planning system may be utilized to develop a patient-specific surgical plan in which the locations of one or more surgical tools, implants, cutting planes, drilling axes, etc. may be determined preoperatively so as to achieve one or more goals of the surgical procedure. Additionally or alternatively, the surgical plan may further include planned modifications to an anatomical structure, e.g., reshaping a bone surface. The surgical planning system may generate one or more computer-generated models of a portion of a patient's anatomy, such as surface models, based on shape data for the patient from an imaging study. The surgical planning system may establish one or more coordinate systems. The locations of the surgical tools, implants, cutting planes and/or drilling axes and the modifications to the anatomical structures may be planned relative to the one or more coordinate systems. In some embodiments, a location of the registration and tracking device(s) may also be determined relative to the portion of the patient's anatomy and to the one or more coordinate systems. In some embodiments, the locations of the surgical tools, implants, cutting planes and/or drilling axes and the modifications to the anatomical structures may be translated to a coordinate system for the registration and tracking device(s). The planning system may generate images of various combinations of one or more of the patient's anatomy, the registration and tracking device(s), the surgical tools, the implants, the cutting planes and/or the drilling axes at the planned locations, and the anatomical structures as modified. The planning system may convert the images into a format for presentation as holograms by the MR-HMD.

The MR-HMD may utilize image and/or object recognition to recognize the registration and tracking device(s), an image associated with the registration and tracking device(s), and/or a portion of the patient's anatomy to register the patient to the preoperatively generated holograms. For example, with the patient on an operating table in the operating room, the registration and tracking device(s) may be docked to the patient in the planned location (or affixed in a random location). The MR-HMD may detect and track the registration and tracking device(s) during at least a portion of the surgical procedure. The MR-HMD may present the holograms and anchor them to the patient based on the coordinate system for the registration and tracking device(s). The surgeon may utilize the holograms as visual guides during the surgical procedure. For example, the holograms may be called up and presented in a sequence that follows the steps of the surgical procedure. One or more holograms may present a surgical tool in a planned location, such as a drill bit for an optimal screw placement. The surgeon may manually position the physical surgical tool to be aligned with the surgical tool of the hologram. One or more holograms may present an anatomical structure modified in a planned manner. The surgeon may modify the physical anatomical structure to match the holograms. By using the holograms as guides for operating surgical tools, modifying anatomical structures and/or inserting implants, the surgeon may achieve the one or more goals of the surgical procedure.

In some embodiments, the systems and methods do not perform intraoperative imaging of the patient and do not track surgical tools or implants during the surgical procedure. In other embodiments, the systems and methods may additionally track one or more surgical tools or implants during the surgical procedure.

The present disclosure provides systems and methods for planning the trajectories of screws used to secure an acetabular cup component within the acetabulum of a patient's pelvis and for maximizing the lengths of the screws. The systems and methods also guide a surgeon, during the surgical procedure, to install the screws of the planned lengths along the planned trajectories. In some embodiments, the trajectories and lengths of the screws may be determined based on the placement of the acetabular cup component within the acetabulum. There may be several variables to consider when implanting the shell portion of an acetabular cup component, such as the position of the shell within the acetabulum, the orientation of the central axis of the shell, and the rotation of the shell about its central axis. For example, the cup may have a central axis, and this axis may be oriented relative to a coordinate frame, such as the Anterior Pelvic Plane (AP Plane), the Anterior Posterior Plane, etc. An exemplary default orientation of a cup may be 29 degrees of operative anteversion and 40 degrees of operative inclination relative to the AP Plane. The cup may then be rotated about, e.g., around, its central axis, without changing its orientation relative to the AP Plane. The cup also may have an x,y,z position within the acetabulum. For example, the x,y,z position of the center of the sphere defined by the cup may be moved toward the midline of the patient's medial-lateral axis, e.g., to get better bony coverage. In other cases, the cup's x,y,z, position may be raised within the native acetabulum, for example to make sure there is good contact between the cup and the bone superiorly. Other times, the cup's x,y,z position may be moved down within the native acetabulum to bring the cup into a more normal position. The cup's x,y,z position may be calculated relative to the raw coordinate system of the CT study. In other embodiments, the cup's x,y,z position may be calculated relative to the AP Plane coordinate system or any other coordinate system such as a functional coordinate system related to how the patient holds their pelvis in a standing or lying position for example. With respect to the cup's orientation, the relationship between the spine and the pelvis of the patient may be considered such that overall cup orientation relative to the AP Plane coordinate system may be individualized, e.g., it may be patient-specific.

The orientation and x,y,z position components of cup placement may be determined by factors independent of those for optimizing screw location, screw orientation and screw length. The cup's rotation component, however, may be changed without altering the overall orientation and x,y,z, position of the cup component. Changing the cup's rotation, moreover, can result in optimization of screw fixation, such as maximizing screw length as compared to leaving the rotation component unchanged.

### BRIEF DESCRIPTION OF THE DRAWINGS

The description below refers to the accompanying drawings, of which:

FIG. 1 is a schematic illustration of an operating room in accordance with one or more embodiments;

FIG. 2 is a schematic illustration of a Mixed Reality (MR) device in accordance with one or more embodiments;

FIG. 3 is a pictorial, perspective, exploded view of an MR device in accordance with one or more embodiments;

FIG. 4 is a pictorial representation of a surgical procedure showing a registration and tracking device docked on a patient in accordance with one or more embodiments;

FIG. 5 is an illustration of a 3D surface model of a pelvis with a model of the registration and tracking device docked thereto in accordance with one or more embodiments;

FIG. 6 is a schematic illustration of an image projected by an MR device showing a virtual image of the patient's pelvis underneath the skin from the exact same perspective as the surgeon at that moment in accordance with one or more embodiments;

FIG. 7 is a pictorial representation of the view into the acetabulum of a patient through an incision during surgery in accordance with one or more embodiments;

FIG. 8 is an illustration of a 3D surface model of the patient's pelvis from the same perspective as FIG. 7 in accordance with one or more embodiments;

FIG. 9 is a schematic illustration of an image projected by an MR device showing a virtual image of the patient's pelvis underneath the skin from the exact same perspective as the surgeon at that moment in accordance with one or more embodiments;

FIG. 10 is a schematic, functional illustration of an example navigation system in accordance with one or more embodiments;

FIG. 11 is a schematic illustration of an example surgical planning system in accordance with one or more embodiments;

FIG. 12 is an illustration of a planning window in accordance with one or more embodiments;

FIG. 13 is an illustration of a planning window in accordance with one or more embodiments;

FIG. 14 is a pictorial representation of a hologram in accordance with one or more embodiments;

FIG. 15 is a pictorial representation of a portion of a registration and tracking tool in accordance with one or more embodiments;

FIG. 16 is a perspective view of a portion of a 3D model of a tool in accordance with one or more embodiments;

FIG. 17 is an illustration of a planning window in accordance with one or more embodiments;

FIG. 18 is a pictorial representation of a hologram co-located with a physical object in accordance with one or more embodiments;

FIG. 19 is a pictorial representation of a hologram in accordance with one or more embodiments;

FIG. 20 is a pictorial representation of a hologram in accordance with one or more embodiments;

FIG. 21 is a pictorial representation of a hologram in accordance with one or more embodiments;

FIG. 22 is a pictorial representation of a hologram in accordance with one or more embodiments;

FIG. 23 is a pictorial representation of a hologram in accordance with one or more embodiments;

FIG. 24 is an illustration of an example planning window for a portion of a surgical plan in accordance with one or more embodiments;

FIG. 25 is an illustration of a planning window in accordance with one or more embodiments;

FIG. 26 is an illustration of a planning window in accordance with one or more embodiments;

FIG. 27 is an illustration of a planning window in accordance with one or more embodiments;

FIG. 28 is an illustration of a planning window in accordance with one or more embodiments;

FIG. 29 is an illustration of a planning window in accordance with one or more embodiments;

FIG. 30 is a pictorial representation of an example 2D CT image set of a patient's pelvis in accordance with one or more embodiments;

FIG. 31 is a pictorial representation of an example 2D CT image set of a patient's pelvis in accordance with one or more embodiments;

FIG. 32 is a pictorial representation of an example 2D CT image set of a patient's pelvis in accordance with one or more embodiments;

FIG. 33 is a partial side view of a patient's acetabulum with a custom fitted template in accordance with one or more embodiments;

FIG. 34 is a perspective view of a portion of a registration and tracking tool in accordance with one or more embodiments;

FIG. 35 is an illustration of a surface model of a pelvis with three cut planes in accordance with one or more embodiments;

FIG. 36 is an illustration of a surface model of a pelvis with three cut planes in accordance with one or more embodiments;

FIG. 37 is a pictorial representation of an image generated and projected by an MR device in accordance with one or more embodiments;

FIG. 38 is an illustration of a surface model of a pelvis illustrating viewpoints of a surgeon in accordance with one or more embodiments;

FIG. 39 is a pictorial representation of an image generated and projected by an MR device in accordance with one or more embodiments;

FIG. 40 is a pictorial representation of an image generated and projected by an MR device in accordance with one or more embodiments;

FIG. 41 is a schematic illustration of an operating room in accordance with one or more embodiments;

FIG. 42 is a pictorial representation of a surgical scene as viewed through an MR device in accordance with one or more embodiments;

FIG. 43 is a schematic illustration of a front view of a pelvis in accordance with one or more embodiments;

FIG. 44 is an illustration of an example planning window that may be generated by the surgical planning system and presented on the display in accordance with one or more embodiments;

FIG. 45 is an illustration of an example planning window that may be generated by the surgical planning system and presented on the display in accordance with one or more embodiments;

FIG. 46 is an illustration of an example planning window that may be generated by the surgical planning system and presented on the display in accordance with one or more embodiments;

FIG. 47 is an illustration of an example planning window that may be generated by the surgical planning system and presented on the display in accordance with one or more embodiments;

FIG. 48 is an illustration of an example planning window that may be generated by the surgical planning system and presented on the display in accordance with one or more embodiments;

FIG. 49 is an illustration of an example planning window that may be generated by the surgical planning system and presented on the display in accordance with one or more embodiments;

FIG. 50 is a pictorial representation of an example hologram in accordance with one or more embodiments;

FIG. 51 is a pictorial representation of another example hologram in accordance with one or more embodiments;

FIG. 52 is a pictorial representation of a surgical scene as viewed by a surgeon through an MR device, such as a see-through HMD, in accordance with one or more embodiments;

FIG. 53 is another pictorial representation of a surgical scene as viewed by a surgeon through an MR device, such as a see-through HMD, in accordance with one or more embodiments;

FIG. 54 is yet another a pictorial representation of a surgical scene as viewed by a surgeon through an MR device, such as a see-through HMD, in accordance with one or more embodiments;

FIG. 55 is an illustration of an example planning window that may be generated by the surgical planning system and presented on the display in accordance with one or more embodiments;

FIG. 56 is an illustration of an example planning window that may be generated by the surgical planning system and presented on the display in accordance with one or more embodiments;

FIG. 57 is an illustration of an example sequence of images representing at least part of a surgical plan in accordance with one or more embodiments; and

FIG. 58 is an illustration of an example sequence of images representing at least part of a surgical plan in accordance with one or more embodiments.

### DETAILED DESCRIPTION OF ILLUSTRATIVE EMBODIMENTS

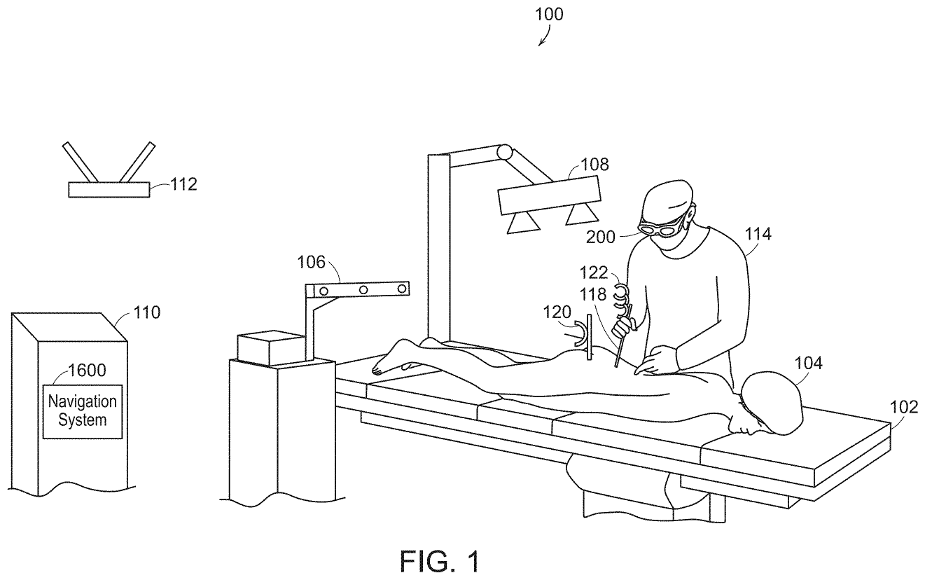

FIG. 1 is a schematic illustration of an operating room 100 in accordance with one or more embodiments. Disposed in the operating room 100 is an operating table 102 on which a patient 104 is positioned for a surgical procedure. Also disposed in the operating room 100 are a tracking system 106, a data processing device 110, and a network device, such as a wireless router 112. A surgeon 114 may be in the operating room. The surgeon 114 may be wearing a mixed reality (MR) device 200, such as a head mounted device (HMD). Optionally, a three-dimensional (3D) detection system 108 may be disposed in the operating room. Exemplary 3D detection systems include stereoscopic camera systems, Structured Light imaging systems, and Continuous-Wave (CW) Time of Flight (ToF) imaging systems, such as the Azure Kinect Developer Kit (DK) from Microsoft Corp. of Redmond, WA, which includes an integrated depth camera, color photo/video camera, inertial measurement unit (IMU), and microphone array. The tracking system 106 may implement infrared, inertial, or other tracking techniques. The 3D detection system 108 may capture images or reflections from objects in the visible or invisible light range. Images generated by the 3D detection system 108 may be used in embodiments when the MR device 200 includes only a single camera or no cameras. The surgeon 114 may manipulate one or more surgical tools, such as surgical tool 118. In some cases, one or more trackers, such as tracker 120, may be attached to anatomical points of the patient 104. Another tracker 122 may be attached to the surgical tool 118. In some embodiments, the data processing device 110 may host and run some or all of the components of a navigation system 1000. In some embodiments, some or all of the components of the navigation system 1000 may be run by the MR device 200.

In some embodiments, other persons in the operating room 100 may be wearing MR devices and holograms presented on the MR device 200 may be presented on these other MR devices. In some embodiments, one or more display devices may be included in the operating room 100. Images captured by the MR device 200 as well as holograms presented by the MR device 200 may be presented on these display devices and watched by others in the operating room 100 and/or by others observing the surgery.

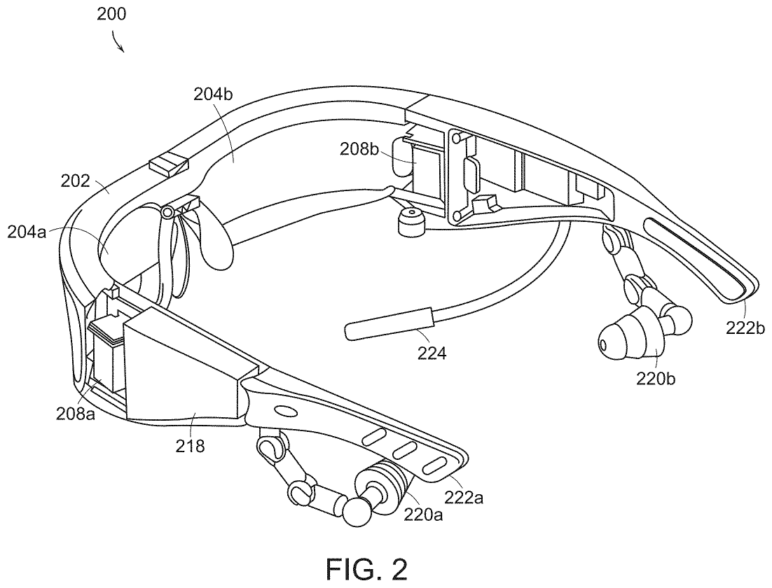

FIG. 2 is a schematic illustration of an example MR device 200 in accordance with one or more embodiments. The MR device 200 may include projection optics suitable to project a virtual image onto a see-through or translucent lens, enabling the surgeon 114 to view the surrounding environment, such as a surgical field, as well as the displayed virtual image. The MR device 200 may include a frame 202 having two lenses 204a and 204b, two arms 222a and 222b, and projectors 208a and 208b, which may be disposed on the front of the MR device 200 or in the arms 222a and 222b, among other places. The projectors 208a and 208b may project virtual images, e.g., holograms, to the user, for example on the lenses 204a and 204b and/or on the user's eyes. The projectors 208a and 208b may be nanoprojectors, picoprojectors, microprojectors, femtoprojectors, LASER-based projectors, or holographic projectors, among others. As noted, the two lenses 204a and 204b are see-through or translucent, although in other embodiments only one lens, e.g., lens 204a may be translucent while the other lens 204b may be opaque or missing. In some embodiments, the MR device 200 may also include two articulating ear buds 220a and 220b, a radio transceiver 218, and a microphone 224. In some embodiments, the MR device 200 may present one or more sounds associated with holograms and may accept voice commands from the user.

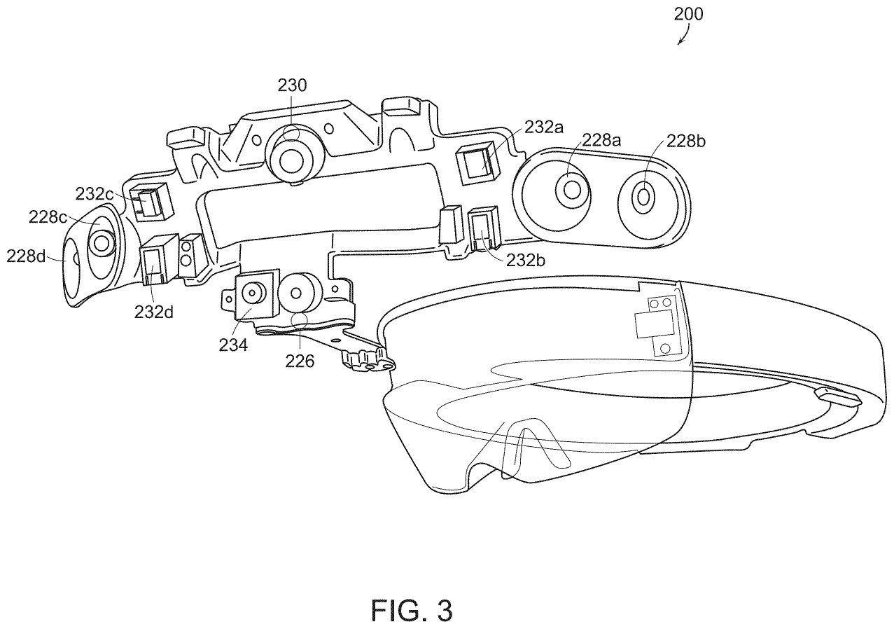

FIG. 3 is a pictorial, perspective, exploded view of the MR device 200 in accordance with one or more embodiments. The MR device 200 may further include a plurality of cameras and/or sensors. For example, in some embodiments, the MR device 200 may include a color video camera 226, four gray-scale cameras 228a-d, and one or more depth cameras or sensors, such as a depth camera 230. The MR device 200 also may include one or more infrared (IR) emitters 232a-d that work together with the depth camera 230 as a Continuous-Wave (CW) Time of Flight (ToF) emitter/receiver. The MR device 200 also may include one or more sensors, such as a light sensor 234. It should be understood that the MR device 200 may include other sensors, such as accelerometers, gyroscopes, resistive sensors, current sensors, piezoelectric sensors, voltage sensors, capacitive sensors, global positioning satellite receivers, compasses, altimeters, rangefinders, thermometers, chemical sensors, eye tracking cameras or sensors, and/or moisture sensors. In some embodiments, one or more of the sensors may sense movement of the surgeon 114, such as when and by how much the surgeon 114 moves, tilts and/or swivels his or her head. For example, a set of sensors may be organized as an Inertial Measurement Unit (IMU).

In some embodiments, 3D information of the wearer's environment may be generated from data output by various combinations of the cameras 226, 228a-d, and 230. For example, various combinations of the cameras 226, 228a-d, and 230 may be configured as stereoscopic cameras, a Structured Light emitter/receiver, or the Continuous-Wave (CW) Time of Flight (ToF) emitter/receiver, among others. Various combinations of the cameras 226, 228a-d, and 230 may be referred to as a spatial detection system.

As described, data output by various combinations of the cameras 226, 228a-d, and 230 included on the MR device 200 may be used to perform registration and/or navigation during one or more surgical procedures. In other embodiments, the MR device 200 may include an infrared stereoscopic tracker. In this case, the MR device 200 may be used to perform infrared stereoscopic tracking of one or more trackers, such as the tracker 120 and/or tracker 122, among others. Additionally, an augmented reality viewpoint may be projected onto the MR device 200.

Suitable MR devices include the HoloLens series of mixed reality devices from Microsoft Corp., the Magic Leap One device from Magic Leap, Inc. of Plantation, FL, and the Blade smart glasses from Vuzix Corp. of West Henrietta, NY, among others, and are described in U.S. Patent Publication No. 2019/0025587 for MR Glasses with Event and User Action Control of External Applications to Microsoft Corp. and U.S. Patent Publication No. 2019/0285897 for Display Device to Apple Inc., which are hereby incorporated by reference in their entireties.

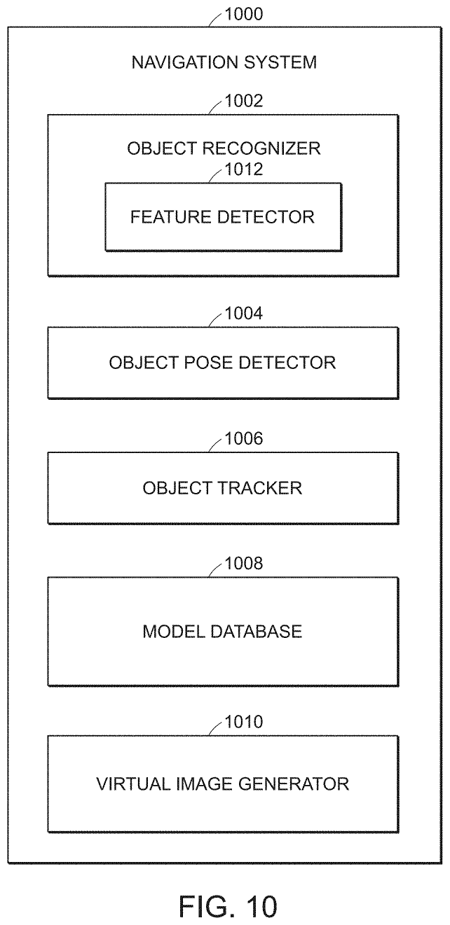

FIG. 10 is a schematic, functional illustration of the navigation system 1000 in accordance with one or more embodiments. The navigation system 1000 may include an object recognizer 1002, an object pose detector 1004, an object tracker 1006, a model database 1008, and a virtual image generator 1010. The object recognizer 1002 may include a feature detector 1012.

It should be understood that the navigation system 1000 is for illustrative purposes only and that the navigation system 1000 may take other forms including additional and/or other components.

One or more of the components of the navigation system 1000 may be implemented using computer vision techniques. Alternatively or additionally, one or more of the components may be implemented using machine learning, such as artificial intelligence (AI), techniques.

In other embodiments, some or all of the components of the navigation system 1000 may be run on the MR device 200, which as noted may include one or more processors and memories. In other embodiments, some or all of the components of the navigation system 1000 may be implemented as a cloud-based service accessible by a client running on the data processing device 110 and/or on the MR device 200. It should be understood that the components of the navigation system 1000 may be implemented in other ways.

Automated Recognition and Registration of Tools and Anatomical Structures: Example: The HipXpert Tool

A patient may be diagnosed with a medical condition that requires surgery. In preparation for the surgical procedure, one or more data gathering procedures may be performed. For example, one or more digital images, such as Computed Tomography (CT), Magnetic Resonance Imaging (MRI), conventional radiographs (X-rays), or ultrasonic images, may be taken of the patient. Specifically, images may be taken of that portion of the patient's anatomy on which the surgery is to be performed. It should be understood that any diagnostic test or measurement, particularly one that improves dimensional understanding about the specific portion of the patient's anatomy to be operated upon, may be performed and used for patient-specific planning.

For example, a patient may be diagnosed with hip joint failure, and may require total hip replacement (THR) surgery either on the left hip, the right hip, or both hips. In this case, one or more CT scans of the patient's hip may be taken. The one or more digital images (CT, radiographic, ultrasonic, magnetic, etc.) may be taken on the day of the patient's preoperative visit, at any time prior to surgery, or even during surgery. The one or more digital images may provide three-dimensional information regarding the surface and/or structure of the patient's hip and associated or adjacent structures.

A surgical planner, such as an experienced surgeon or other person, may utilize a 3D modeling tool of a planning tool to create one or more computer-generated, three-dimensional (3D) models of the patient's anatomy, such as the patient's hip, based on the one more digital images taken of the patient, e.g., CT, MR, or other digital images. Additionally or alternatively to generating a model based on CT, MR, or other digital images, a patient-specific model may be created using predictive modeling, e.g., based on patient-specific characteristics. That is, a statistical shaped model or other predictive model may be created on a patient-specific data input, such as a digital x-ray or a combination of minimum datasets.

The surgical planner may utilize the planning tool to create a surgical plan for the surgical procedure that is to be performed on the patient. For example, the surgical planner may create a plan for implanting one or more prosthetic or surgical components, such as an acetabular cup component, into the patient's hip during THR surgery, using one or more surgical tools. The surgical planner may utilize the planning tool to establish one or more coordinate systems, such as the anterior pelvic (AP) plane coordinate system, based on the 3D computer-generated model of the pelvis. Other patient-specific coordinate systems, for example, for use by the one or more surgical tools, may also be established, for example, by selecting three points on the 3D model of the patient's pelvis, such as an ipsilateral hemipelvic plane coordinate system. Further, “functional” coordinate systems may be established based on the position of a body part in a functional position. For example, a functional coordinate system of the pelvis may be established simply by knowing and accepting the position that the patient's pelvis was in while the imaging was acquired.

In some embodiments, the surgical planner may utilize the planning tool to calculate one or more inputs and/or adjustments to be made on the one or more surgical tools, such as the adjustable HipXpert® tool. The inputs and/or adjustments may be based, at least in part, on information, such as spatial information, derived from the 3D model of the pelvis that was created, on some or all of the patient-specific information, and/or on statistical information known to or accessible by the surgical planner. For example, the inputs and/or adjustments may be used to customize the HipXpert tool to fit, e.g., dock, to the patient's pelvis, such that the predicted docking location of the HipXpert tool would be known relative to any other coordinate system of the pelvis, e.g., the AP plane coordinate system. The surgical planner also may choose particular prosthetic hip components, and may plan their location within the 3D model of the pelvis in order to accomplish a particular goal for the surgery, such as optimizing the changes in leg length, offset, and/or AP position. In some cases, optimizing the changes may mean minimizing changes to leg length, offset, and/or AP position. In other cases, it may mean achieving intended changes to leg length, offset, and/or AP position.

The surgical planner may plan the locations of the selected prosthetic components to achieve the goals. For example, the location of a selected acetabular cup component within the acetabulum may be determined. The location may include the depth of the cup component in the acetabulum and the planning phase may include determining how the acetabulum should be prepared, e.g., shaped, in order to receive the cup component at the planned location. For example, the plan may specify the depth and/or shape of the cup bed of the acetabulum. The location may include the orientation of an axis, e.g., a central axis, of the cup component relative to the AP plane coordinate system.

A version of the 3D model of the pelvis may be generated with the acetabulum prepared to receive the cup component. For example, a 3D model of the cup bed may be generated. Furthermore, in some embodiments, 3D models of the prosthetic components may be included in and/or available to the planning tool. The surgical planner may place a 3D model of the cup component at the planned location in the 3D model of the pelvis. Similarly, a 3D model of a selected femoral stem may be placed at the planned location in the 3D model of the hip.

In some embodiments, the HipXpert tool may include a guide, such as a rod. The surgical planner may determine one or more adjustments to the HipXpert tool so that, when it is docketed to the patient's pelvis, the guide will point in the direction of acetabular cup orientation, as planned.

The surgical plan may thus include instructions for setting up and using one or more surgical tools during the procedure. In other embodiments, the surgical plan may be or may include machine instructions, such as executable code, for operating one or more tools or devices, such as a surgical tool or a machine, to assist during the surgical procedure. In some embodiments, the surgical plan may include machine instructions to be executed by a robotic surgical tool that will perform all or part of the procedure. In addition to controlling a surgical robot, the surgical plan may provide instructions for controlling a free-hand surgical device, such as a rotating tool, to turn on when it is in a location where cutting is to be performed and either turn off or disable cutting, e.g., through deployment of a protective sheath, when it is in a location where cutting should not take place.

Exemplary surgical robots include the surgeon-controlled robotic arms from Mako Surgical Corp. of Fort Lauderdale, FL. Exemplary free-hand tools include the freehand sculptor from Blue Belt Technologies, Inc. of Pittsburgh, PA.

Nonetheless, it should also be understood that in some embodiments the surgical plan may be developed and/or revised during the surgical procedure while in other embodiments no explicit surgical plan may be created.

Manual Registration of Holograms: Example: The HipXpert Tool

As described, during a planning stage, an AP Plane coordinate system may be defined for a 3D surface model of a patient's pelvis or portion thereof. In some embodiments, a first 3D surface model may include a portion of one or more of the patient's femurs including the femoral heads in the hip joints. A second 3D surface model may omit the patient's femurs and only include the pelvis or a portion thereof. In some embodiments, a femoral coordinate system and/or a tibial coordinate system may also be defined in addition to the AP Plane coordinate system.

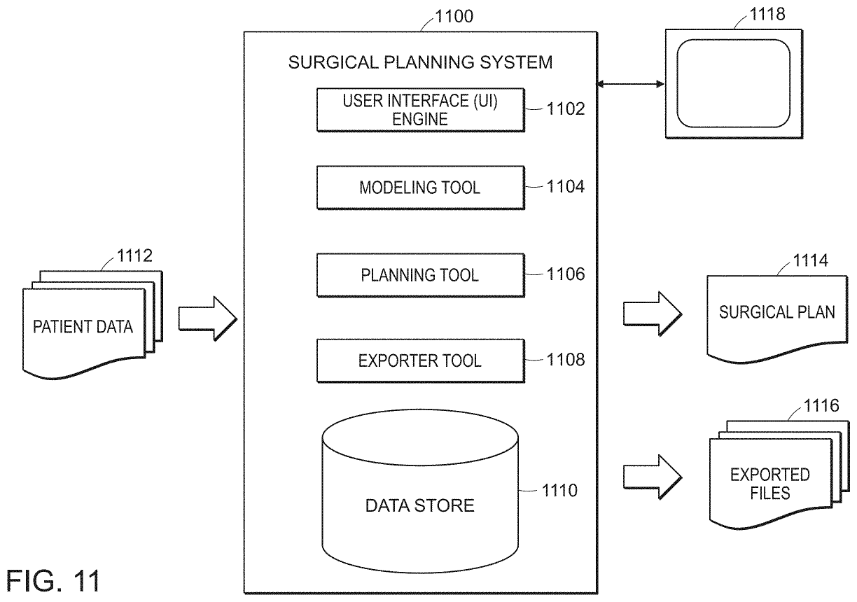

FIG. 11 is a schematic illustration of an example surgical planning system 1100 in accordance with one or more embodiments. The surgical planning system 1100 may include a user interface (UI) engine 1102, a modeling tool 1104, a planning tool 1106, an exporter tool 1108, and a data store 1110. The surgical planning system 1100 may receive patient data, as indicated at 1112, which may include volume or shape data in the form of magnetic resonance imaging (MRI) data, computed tomography (CT) data, simultaneous biplanar radiography data, conventional plain radiograph data, ultrasonic data, and/or other data of a patient's hip or other anatomical structure. The surgical planning system 1100 may create one or more electronic surgical plans, such as plan 1114, for the hip surgery, and may export one or more files, e.g., for generating holograms, as indicated at 1116. The surgical planning system 1100 may include or have access to a display 1118.

Suitable tools for generating 2D and/or 3D displays of anatomical structures from volume or shape data include the OsiriX image processing software from Pixmeo SARL of Bernex Switzerland, the TraumaCad pre-operative planning system, the MAKOplasty Total Hip Application pre-operative and intra-operative planning system, and the HipXpert Navigation System Application 1.4.0. Nonetheless, those skilled in the art will understand that other image processing software may be used.

One or more of the patient data 1112, the surgical plan 1114, and the exported files 1116 may be implemented through one or more data structures, such as files, objects, etc., stored in the electronic memory of a data processing device, such as the data store 1110.

As noted, the surgical planner may select one or more prosthetic components to be used in a surgical procedure, such as a prosthetic cup component and/or a femoral stem component and plan their placement in the patient's body. The plan for the prosthetic cup component may include a planned location, including a depth and an orientation within the acetabulum. The plan may also include the shape of the cup bed to receive the cup component. For the femoral stem component, the plan may define the location of the femoral stem component within the femur and its orientation relative to the femoral coordinate system and/or tibial coordinate system.

In some embodiments, the plan may incorporate 3D models of one or more other tools, such as the HipXpert tool, acetabular reamers and cup impactors, among others.

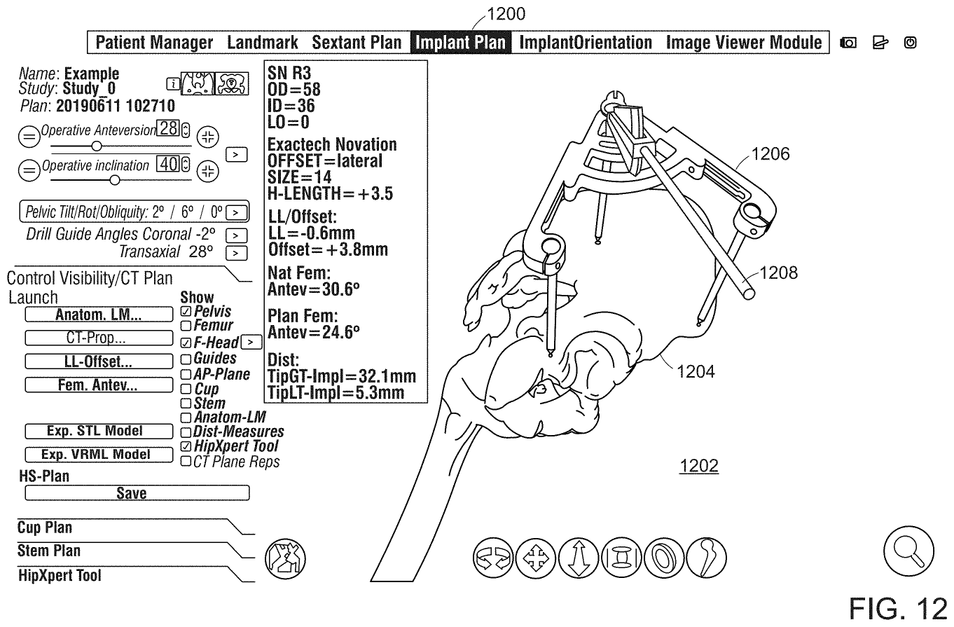

FIG. 12 is an illustration of a planning window 1200 generated by the surgical planning system 1100 and presented on the display 1118 in accordance with one or more embodiments. The planning window 1200 includes a model pane 1202 presenting a 3D model of the patient's pelvis 1204. Docked to the model of the pelvis 1204 is a 3D model of the HipXpert tool 1206. As noted, the model of the HipXpert tool 1206 may include a guide, such as a rod 1208. If utilized, the planner may determine one or more adjustments to the HipXpert tool so that when it is docked to the patient's pelvis the rod 1208 points in the direction of acetabular cup orientation, as planned.

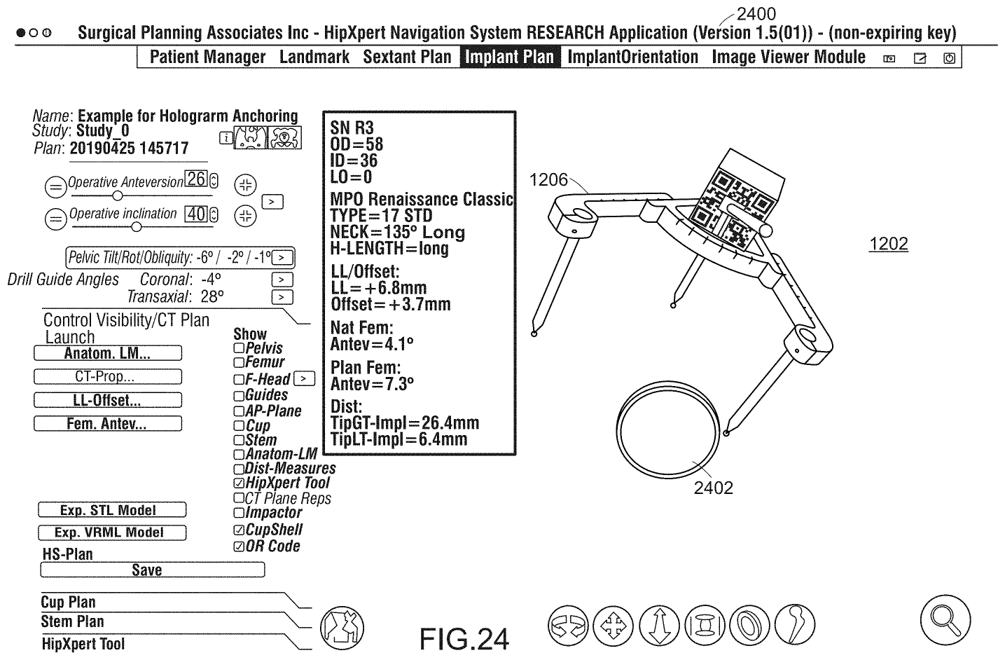

The surgical planner may plan the position, shape and orientation of the cup bed to receive the prosthetic cup component. FIG. 24 is an illustration of an example planning window 2400 for a portion of a surgical plan in accordance with one or more embodiments. The planning window 2400 also includes the model pane 1202 presenting the 3D model of the HipXpert tool 1206. A 3D model of a cup bed 2402 as planned may also be presented in the model pane 1202. The 3D model of the patient's pelvis appearing in other planning windows may be omitted in the planning window 2400 for the cup bed 2402. The surgical planner may plan the position, shape and orientation of the cup bed 2402 to achieve the goals of the surgery. The cup bed refers to the ideal surgically created bone surface to receive the prosthetic cup component in the planned location.

In some embodiments, the surgical planner may determine the location of the acetabular reamer at the 3D model of the pelvis, e.g., relative to the AP Plane coordinate system, to prepare the cup bed as planned. For example, the acetabular reamer may have a handle defining a longitudinal axis. The surgical planner may position a 3D model of the acetabular reamer so that the cutting basket of the reamer is positioned in the acetabulum to prepare the cup bed as planned in position and orientation.

The surgical planner also may determine the location of the cup impactor at the 3D model of the pelvis, e.g., relative to the AP Plane coordinate system, to implant the cup component in the cup bed as planned. For example, the cup impactor may have a handle defining a longitudinal axis. The surgical planner may position a 3D model of the cup impactor so that the longitudinal axis defined by the handle positions the cup component at the end of the cup impactor in the cup bed as planned.

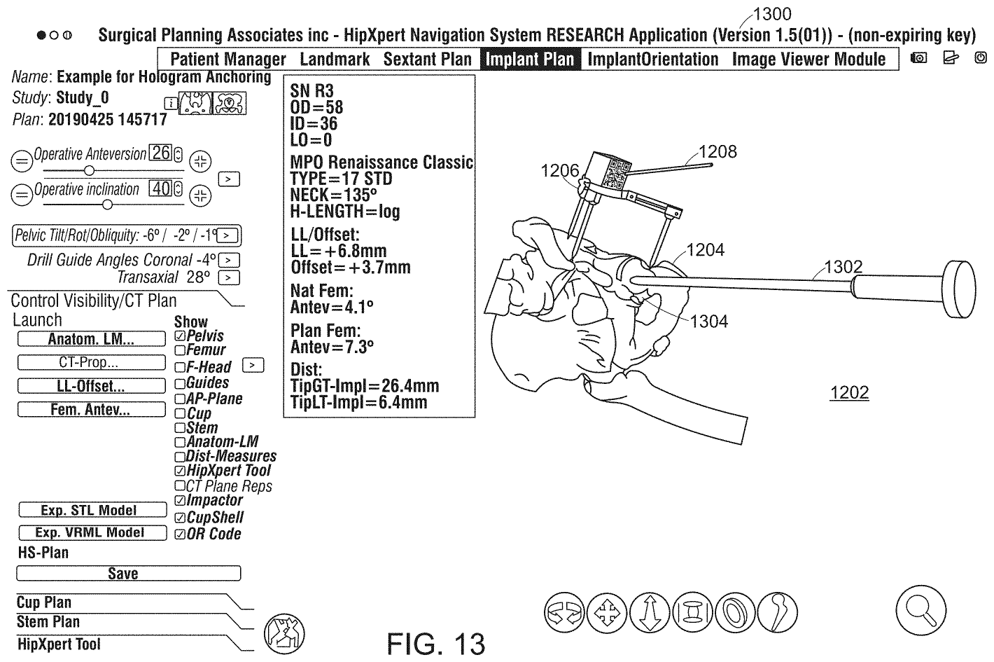

FIG. 13 is an illustration of an example planning window 1300 generated by the surgical planning system 1100 for a portion of a surgical plan and presented on the display 1118 in accordance with one or more embodiments. The planning window 1300 also includes the model pane 1202 presenting the 3D model of the patient's pelvis 1204 and the 3D model of the HipXpert tool 1206. A 3D model of a cup impactor 1302 and a 3D model of a prosthetic cup component 1304 may also be presented in the model pane 1202. The surgical planner may position the model of the cup component 1304 seated in the cup bed at the planned location and orientation. In addition, the surgical planner may position the model of the cup impactor 1302 at the location for implanting the cup component 1304 at the planned position and orientation.

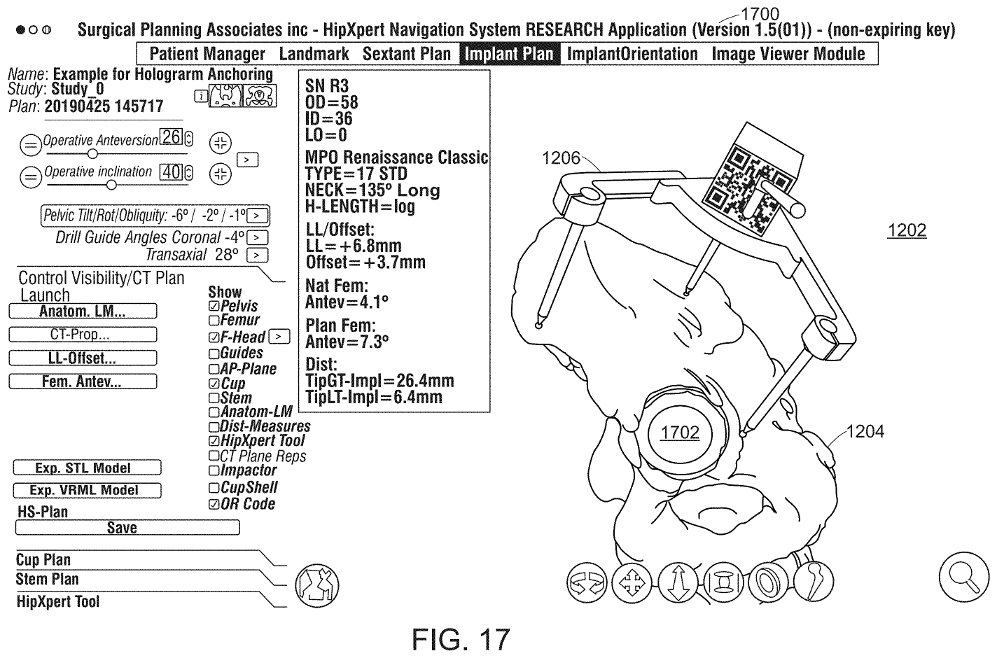

FIG. 17 is an illustration of an example planning window 1700 for a portion of a surgical plan generated by the planning system 1100 in accordance with one or more embodiments. The planning window 1700 includes the 3D model of the patient's pelvis 1204 and the 3D model of the HipXpert tool 1206. The planning window 1700 further includes a 3D model of a cup component and liner 1702 as implanted in the acetabulum at a desired location, for example relative to the AP Plane coordinate system.

In some embodiments, the plan may also include one or more tracking devices attached to the patient's pelvis whose location is defined relative to the AP Plane coordinate system or another coordinate system. The one or more tracking devices may include a weathervane type device that may be planned to point in the orientation defined for the central axis of the prosthetic cup component.

In some embodiments, the plan may include files of 3D models of one or more of:
the patient's pelvis (or portion thereof);
the patient's femur(s) (both alone and as part of the pelvis);
the HipXpert tool as customized for the patient (both alone and as positioned on the patient's pelvis);
a reamer tool positioned at the planned depth of the acetabulum and in the planned orientation for the cup component relative to the AP Plane coordinate system (or a sequence of reamer tools with different size cup reamers leading to a final one);
a hemispherical surface representing the exact position of the ideally prepared bone surface for receipt of the acetabular component;
a cup impactor tool at the planned position and orientation relative to the AP Plane coordinate system for the cup component;
the selected prosthetic cup component at the planned orientation and depth in the acetabulum relative to the AP Plane coordinate system;
the selected prosthetic cup component and liner at the planned orientation and depth in the acetabulum relative to the AP Plane coordinate system;
the prosthetic stem at the planned orientation and depth relative to the femoral coordinate system, and/or the tibial coordinate system; and/or
the one or more tracking devices, e.g., weathervane.

It should be understood that various combinations of the above-listed 3D models also may be created.

As described, by anchoring the holograms, the systems and methods do not have to track any of the surgical tools, e.g., the systems and methods may be free of tracking surgical tools. Instead, the surgeon can track the instruments using his or her eyes to bring the instruments in line with the corresponding anchored holograms. Nonetheless, in some embodiments, the systems and methods may track one or more of the surgical tools.

The planning tool 1106 may export at least some of these 3D model files into a format compatible with the MR device 200 so that the MR device 200 may project holograms corresponding to the exported 3D model files. For example, one or more of the files representing the 3D objects may be exported and loaded into the memory of the MR device 200. Alternatively, the files representing the 3D objects may be stored at a server and the MR device 200 may be configured as a client capable of accessing those files from the server.

For hip surgery, the following sequence of holograms may be generated:
1. A hologram of the HipXpert tool and the pelvis;
2. A hologram of the HipXpert tool, the pelvis, and the ideal acetabular cup bed;
3. A hologram of the HipXpert tool and the ideal cup bed without showing the pelvis;
4. A hologram of the HipXpert tool, the pelvis, the ideal cup bed or the cup component, and the acetabular cup component impaction handle situated in the ideal orientation for implanting the cup component;
5. A hologram of the HipXpert tool, the pelvis, and the metal acetabular cup component without the bearing insert in which the native pelvis has all osteophytes still in place, and
6. A hologram of the HipXpert tool, the pelvis, the metal acetabular component, and the bearing insert.

Nonetheless, it should be understood that other and/or additional holograms may be generated and included. Exemplary additional holograms include holograms of the acetabular reamer handle and each sequential reamer basket in the ideal location. When the surgeon places the actual reamer handle with the final reamer basket in exact overlap with the hologram of the same, then the cup preparation bed is in the planned place. Such additional holograms may have some advantages over above-described holograms 2 and 3 since the surgeon may be unable to see where the reamer is in space when preparing the bony cup bed. Using those holograms, the surgeon may have to ream, take the reamer out, and look into the incision to compare the real prepared bony cup bed surface to the hologram. If instead or in addition there is a hologram of the exact reamer handle and basket, the surgeon will be able to tell if the cup bed is correct by looking at overlapping holograms and reality mostly outside of the patient's body. This may be more convenient, among other advantages. Also, during cup impaction, instead of the above-described hologram 4 with an idealized straight cup impactor (for alignment only), there may be a hologram of the same exact planned cup impactor to be used in surgery with the same exact planned cup component also to be used in surgery. Then, when impacting the cup, the surgeon can line up not only the orientation of the cup component to be correct, but can also tell if the cup component is fully seated and if it is in the correct place.

In some embodiments, computer-generated, three-dimensional (3D) models, such as other Computer Aided Design (CAD) models, of one or more surgical tools may be stored in the data store 1110. 3D surface models of the surgical tools may be generated from these models and also stored in the data store 1110. In some embodiments, only the 3D surface models may be included in the data store 1110. In some embodiments, 3D surface models of one, a handful or some other small number of standard surgical tools, such as a standard acetabular reamer with a standard cutting basket and a standard acetabular cup impactor may be included in the data store 1110. Holograms that include a reamer or cup impactor may be based on these surface models of a standard reamer or cup impactor.

However, in other embodiments, 3D surface models for actual reamers and/or cup impactors including entire product families from one or more manufacturers, e.g., Stryker Corp. of Kalamazoo, MI, Greatbatch, Inc. (now Integer Holdings Corp.) of Plano, TX, Ortho Solutions UK Ltd. of Essex, UK, Zimmer Biomet Holdings, Inc. of Warsaw, IN, Depuy Synthes of Raynham, MA, etc., may be included in the data store 1110. Furthermore, 3D surface models for different sizes of cutting baskets and different sizes of acetabular cups may be included in the data store 1110. During the surgical planning phase, 3D surface models corresponding to the particular reamer and the particular cup impactor that the surgeon will be using in the surgery may be selected from the data store 1110 and used in creating the surgical plan. 3D models for cup impactors and cup components may even include spatial assembly information for how each of the planned cup assembles onto the cup impactor, e.g., due to thread depth and shell thickness). In this way, holograms representing the particular surgical tools that the surgeon is using may be generated and presented. Furthermore, a sequence of holograms of a reamer with different basket sizes may be generated to indicate the bone cutting work performed by each reamer basket size before moving to a next reamer basket size. The sequence of holograms may illustrate being moved deeper into the acetabulum as further cutting is performed. That is, each hologram may indicate the exact amount of cutting to be performed by each reamer basket size. Additionally, a hologram of a cup impactor and cup that corresponds to the physical cup component being implanted may be generated.

Prior to the surgical procedure, the navigation system 1000 or one or more portions thereof may be loaded into the memory of the MR device 200 and/or made accessible to the MR headset 200. For example, the MR device 200 may be configured as a client of the navigation system 1000, which may be loaded on and run at a server, such as a laptop computer, that is in communicating relationship with the MR device 200. In some embodiments, the planning tool 1106 used to plan the surgery may be loaded and run on the MR device 200.

During the procedure, the surgeon may adjust a physical HipXpert tool as provided in the plan to customize the tool to fit to the patient's pelvis. The surgeon may then place the physical HipXpert tool on the patient's pelvis. The patient may be positioned on an operating room table. The surgeon may wear the MR device 200. The surgeon may control the MR device 200 to render a hologram of the HipXpert tool attached to a hologram of the patient's pelvis as planned. The surgeon may operate user interface elements provided by the MR device 200 to resize, move, and/or rotate the hologram of the HipXpert tool/pelvis so that the hologram is co-located with the physical HipXpert tool attached to the patient's pelvis, e.g., aligned together. More specifically, while the pelvis may not be visible to the surgeon because it is below the patient's skin, the HipXpert tool, which is docked to the patient's pelvis, is visible to the surgeon. Accordingly, the surgeon may resize, move, and/or rotate the hologram of the HipXpert tool/pelvis until it is co-located with the physical HipXpert tool docked to the patient's pelvis. The hologram of the patient's pelvis will also be co-located with the patient's pelvis even though the patient's pelvis is not visible to the surgeon. Once the hologram of the HipXpert tool/pelvis is co-located with the physical HipXpert tool, the surgeon may peg or anchor the hologram of the HipXpert tool/pelvis at that location within the operating room. For example, the MR device 200 may include an anchoring feature for holograms rendered by the MR device 200. In addition, as described herein, in some embodiments, the navigation system 1000 may automatically co-locate one or more of the holograms with reality, for example using image recognition of an image, such as a QR code, or using object recognition of the HipXpert tool as adjusted specifically for the patient.

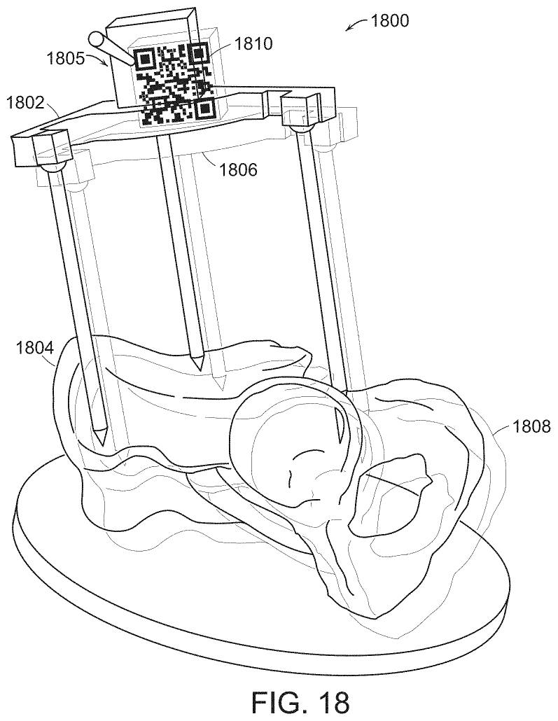

FIG. 18 is a pictorial representation indicated generally at 1800 of a hologram being co-located with a physical object in accordance with one or more embodiments. The representation 1800 includes a physical HipXpert tool 1806 docked to a physical hip model 1808 as planned. The representation 1800 further includes a hologram indicated generally at 1805 that includes a hologram of a HipXpert tool 1802 and a hologram of a hip model 1804 in which the HipXpert tool hologram 1802 is docked to the hologram of the hip model 1804 in the planned manner. The physical HipXpert tool 1806 includes a QR code 1810. The hologram 1805 may be repositioned in space either manually by the wearer of the MR device 200 and/or automatically by the MR device 200 until it is co-located with the physical HipXpert tool 1806. For purposes of explanation, the pictorial representation 1800 shows the physical hip model 1808. However, a patient's hip will not be visible to the surgeon as it is beneath the patient's skin. In some embodiments, the surgeon may manually reposition the hologram 1805 so that the HipXpert tool hologram 1802 is co-located with the physical HipXpert tool 1806, which is visible to the surgeon. While the patient's physical hip is not visible to the surgeon, the hip hologram (illustrated by the hip model hologram 1804) shows the surgeon where the patient's physical hip is. In other embodiments, the object recognizer 1002 may detect the QR code 1810 on the physical HipXpert tool 1806 and automatically co-locate the hologram 1805 to the physical HipXpert tool 1806. Not only may the object recognizer 1002 perform image recognition, such as with a QR code, it may also perform object recognition of the HipXpert tool 1806 itself or the HipXpert tool 1806 plus the actual bony acetabulum.

In some embodiments, the physical HipXpert tool may not include a guide rod. Nonetheless, the surgeon may utilize the guide rod of the hologram of the HipXpert tool to implant the prosthetic cup component in the patient's acetabulum at the planned orientation. That is, the surgeon may use the guide rod of the hologram of the HipXpert tool as a guide for implanting the cup at the planned orientation. Nevertheless, in addition to a hologram of the guide rod (or instead), the MR device 200 may present a hologram of the cup impactor tool, and the surgeon may line up the physical cup impactor tool to this hologram of the cup impactor tool. The surgeon may then manually line up the physical tool with the hologram. As described, in some embodiments, it is not necessary to track the physical tool. Instead, the system may detect one or more of the QR codes of the HipXpert device and anchor the holograms based on the spatial coordinate system exposed by and aligned with the one or more QR codes. The holograms then show the planned locations of the surgical tools, and the surgeon may align the physical tool with the hologram, e.g., the planned location for the tool.

In some embodiments, the surgeon may operate the MR device 200 to render a hologram of the reamer/HipXpert tool/pelvis. The hologram of the reamer may be disposed relative to the hologram of the pelvis such that the hologram of the reamer is at the final position and orientation for preparing the acetabulum to receive the prosthetic cup component relative to the AP Plane coordinate system. The surgeon may operate user interface elements provided by the MR device 200 to resize, move, and/or rotate the hologram of the reamer/HipXpert tool/pelvis so that the hologram is co-located with the physical HipXpert tool attached to the patient's pelvis, e.g., the hologram and the tool are spatially aligned together. The surgeon may operate the MR device 200 to peg or anchor the hologram of the reamer/HipXpert tool/pelvis at that location within the operating room. The surgeon may then operate a physical reamer tool to prepare the acetabulum until the physical reamer is co-located with the hologram of the reamer. For example, the surgeon may position the physical reamer to be co-located with the hologram of the reamer. As noted, the hologram may represent a standard reamer or, in a preferred embodiment, the hologram may represent the particular reamer being used by the surgeon in the surgery, which may make it even easier for the surgeon to line up the physical reamer with the hologram of the reamer. Additionally, a sequence of holograms of reamers, e.g., with different cutting basket sizes, may be presented, and the surgeon may change the physical cutting basket to match the cutting basket included in the hologram. The sequence of holograms also illustrates the depth of cutting to be performed with each cutting basket. When the physical reamer is lined up with the hologram of the reamer, the cutting by the respective cutting basket is complete. The surgeon may change cutting baskets and the next hologram in the sequence may be presented. This process may be repeated until the cup bed is prepared as planned. When the physical reamer (or the physical reamer with the last cutting basket in the case of a sequence of reamers) is co-located with the hologram of the reamer, the cup bed will be prepared for receiving cup component as planned. Suppose for example, the surgical plan call for a 56 mm cup component. The plan may call for a series of reamers, such as a first reamer with a 51 mm basket, a second reamer with a 53 mm basket, a third reamer with a 55 mm basket, and finally a fourth reamer with a 56 mm basket to do a final preparation of the cup bed before putting the cup component in.

The surgeon may operate the MR device 200 to render a hologram of the cup bed/HipXpert tool. The surgeon may operate user interface elements provided by the MR device 200 to resize, move, and/or rotate the hologram of the cup bed/HipXpert tool so that the hologram is co-located with the physical HipXpert tool attached to the patient's pelvis. The surgeon may operate the MR device 200 to peg or anchor the hologram of the cup bed/HipXpert tool at that location within the operation room. The surgeon may look through the incision in the patient and compare the physical acetabulum with the hologram of the cup bed. The surgeon may determine whether the appearance of the physical acetabulum following the reaming matches the hologram of the cup bed. If not, the surgeon may operate the physical reamer to further shape the acetabulum until it matches the hologram of the cup bed.

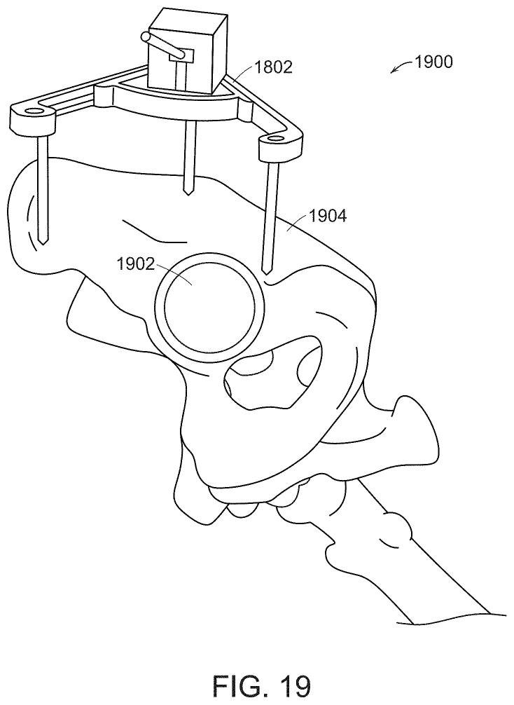

FIG. 19 is a pictorial representation of a hologram 1900 in accordance with one or more embodiments. The hologram 1900 may include the hologram 1802 of the HipXpert device, a hologram 1904 of the patient's pelvis, and a hologram 1902 of the cup bed as planned. During the surgical procedure, the hologram 1900 may be co-located to the corresponding physical objects either manually and/or automatically, for example by co-locating the hologram 1802 of the HipXpert device with the physical HipXpert device. The surgeon may then examine the physical cup bed as prepared, e.g., through the use of the reamer, and see if the shape of the physical cup bed, e.g., depth and center or orientation, matches the hologram 1902 of the cup bed as planned. If not, the surgeon may continue shaping, e.g., using a reamer, the physical cup bed until it matches the hologram 1902.

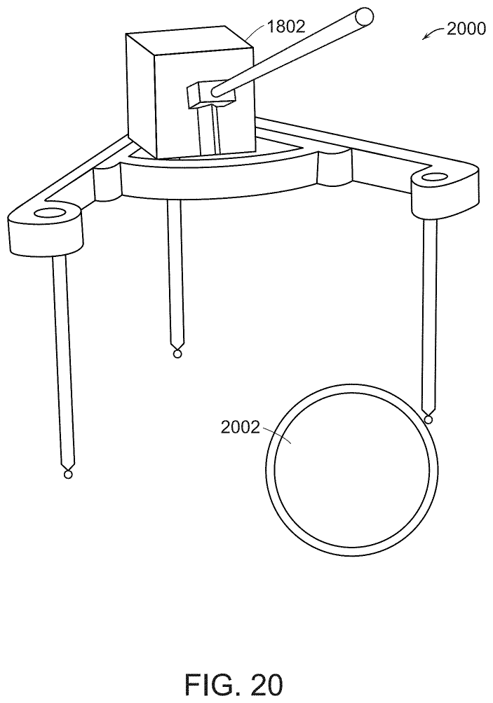

FIG. 20 is a pictorial representation of a hologram 2000 in accordance with one or more embodiments. The hologram 2000 may include the hologram 1802 of the HipXpert device and a hologram 2002 of the prepared cup bed as planned. However, unlike the hologram 1900 (FIG. 19), the hologram 2000 may not include a virtual representation of the patient's pelvis. During the surgical procedure, the hologram 2000 may be co-located to the corresponding physical objects either manually and/or automatically, for example by co-locating the hologram 1802 of the HipXpert device with the physical HipXpert device 1806 (FIG. 18). The surgeon may then examine the physical cup bed as prepared and see if the shape of the physical cup bed, e.g., depth and center or orientation, matches the hologram 2002 of the cup bed as planned. It may be easier for the surgeon to see and compare the physical cup bed with the hologram 2002 of the planned cup bed without a virtual representation of the pelvis as with the hologram 1900, which may interfere with the surgeon's view. Again, if the physical cup bed does not match the shape of the hologram 2002 of the planned cup bed, the surgeon may continue shaping the physical cup bed until it matches the hologram 2002.

Next, the surgeon may operate the MR device 200 to render a hologram of the cup impactor/HipXpert tool/pelvis with the cup impactor disposed at the final location for implanting the prosthetic cup component at the planned orientation and position, e.g., depth, relative to the AP Plane coordinate system. The surgeon may operate user interface elements provided by the MR device 200 to resize, move, and/or rotate the hologram of the cup impactor/HipXpert tool/pelvis so that the hologram is co-located with the physical HipXpert tool attached to the patient's pelvis. The surgeon may operate the MR device 200 to peg or anchor the hologram of the cup impactor/HipXpert tool/pelvis at that location within the operation room.

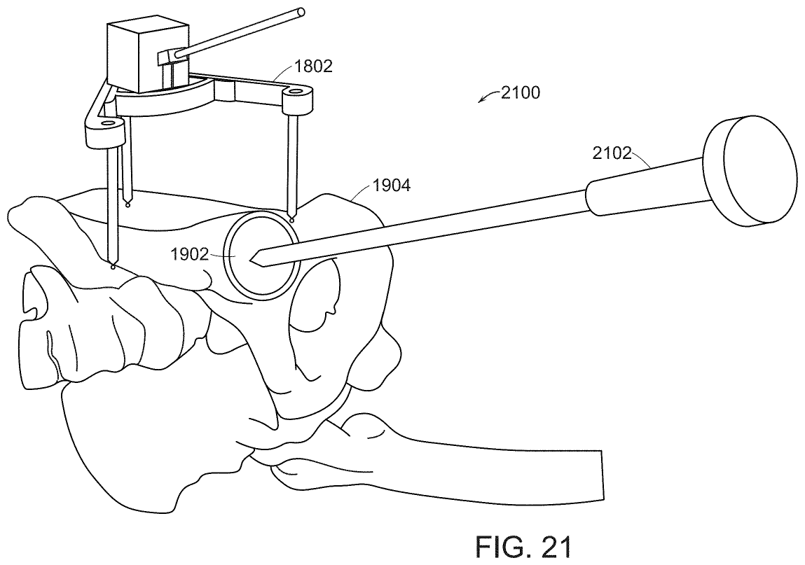

FIG. 21 is a pictorial representation of a hologram 2100 in accordance with one or more embodiments. The hologram 2100 may include the hologram 1802 of the HipXpert device, the hologram 1904 of the patient's pelvis, the hologram 2002 of the cup bed as planned, and a hologram 2102 of a cup impactor disposed at the final location for implanting the prosthetic cup component at the planned orientation and position. During the surgical procedure, the hologram 2100 may be co-located to the corresponding physical objects either manually and/or automatically, for example by co-locating the hologram 1802 of the HipXpert device with the physical HipXpert device.

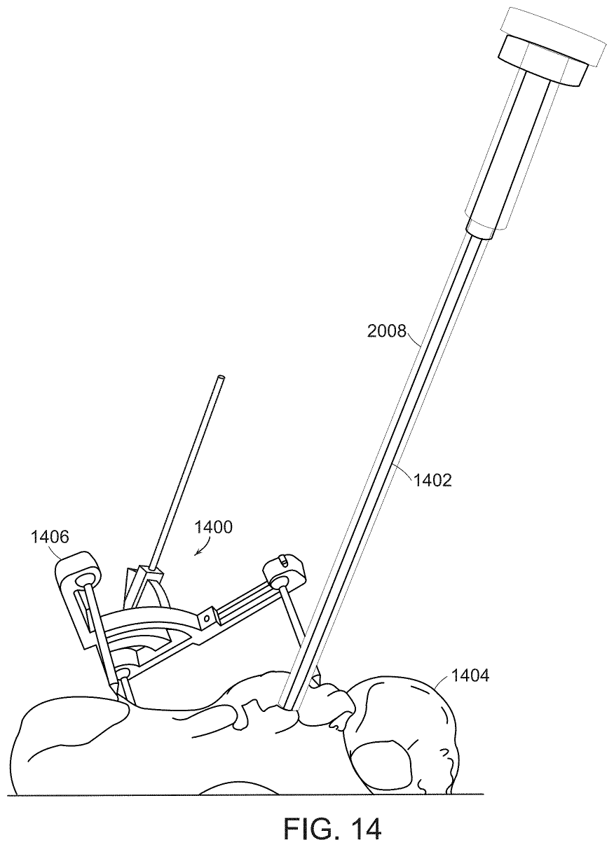

FIG. 14 is a pictorial representation of a hologram 1400 in accordance with one or more embodiments. The hologram 1400 may include a hologram of a pelvis 1404, a hologram of the HipXpert tool 1406, and a hologram of a cup impactor 1408. During the surgical procedure, the hologram 1400 may be positioned such that the hologram of the HipXpert tool 1406 is co-located, e.g., spatially aligned, with the physical HipXpert tool docketed to the patient's pelvis. The surgeon may then use a physical cup impactor 1402 to implant the prosthetic cup component in the cup bed. The surgeon may operate the physical cup impactor 1402 until it is co-located with the hologram 1408 of the cup impactor. When the physical cup impactor 1402 is co-located with the hologram 1408 of the cup impactor, the cup component will be positioned in the cup bed as planned, e.g., at the planned depth and orientation in the acetabulum.

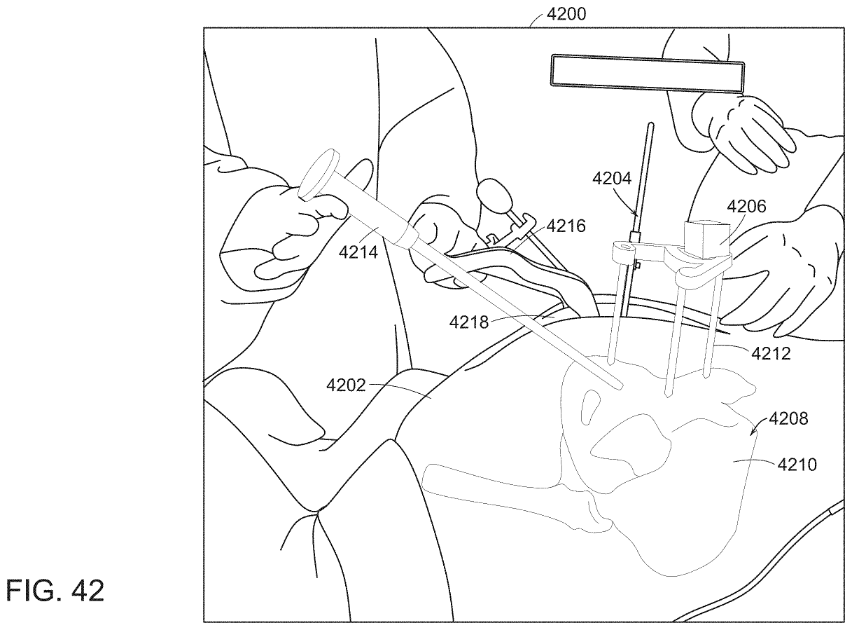

FIG. 42 is a pictorial representation of a surgical scene 4200 as viewed through the MR device 200 in accordance with one or more embodiments. Included in the surgical scene 4200 is a patient 4202. Docked to the patient's pelvis, which is below the skin and not visible, is a three legged registration and tracking device 4204. The registration and tracking device 4204 includes a cube 4206 with QR codes on its surfaces. Also included in the surgical scene 4200 is a hologram indicated generally at 4208 as presented by the MR device 200. The hologram 4208 includes a hologram of the patient's pelvis 4210, a hologram of a registration and tracking device 4212 and a hologram of a cup impactor 4214 at a planned location for implanting a prosthetic cup component. As illustrated, the hologram of the registration and tracking device 4212 is co-located with the physical registration and tracking device 4204, e.g., through image recognition of one or more of the QR codes by the MR device 200 or object recognition of at least a portion of the registration and tracking device 4204. Accordingly, the hologram of the patient's pelvis 4210 is also co-located with the patient's pelvis. A surgeon may position a physical cup impactor 4216 in alignment, e.g., be co-located, with the hologram of the cup impactor 4214. While the hologram of the cup impactor 4214 is straight, the physical cup impactor 4216, which extends into an incision 4218 and is only partially visible, is C-shaped. With the physical cup impactor 4216 positioned in alignment with the hologram of the cup impactor 4214, the surgeon may operate the cup impactor 4216 to implant the cup component disposed at the end of the cup impactor 4216 and thus not visible (except through the incision 4218) at the planned location.

As described, the systems and methods may register the patient's pelvis during surgery with the patient in the operating room. Then, a sequence of holograms may be presented relative to the pelvis as registered. The holograms may include holograms of surgical tools at planned locations and the surgeon may line up physical surgical tools with the holograms to achieve the one or more goals of the surgery. The physical surgical tools do not themselves have to be tracked in the operating room. Nonetheless, in some embodiments, the surgical tools may be tracked, e.g., by the object tracker 1006.

In some embodiments, in addition to presenting static holograms, the MR device 200 may present a sequence of holograms in the form of a holographic movie, which may be paused and resumed by the surgeon as needed during the surgical procedure. The holographic movie may be updated, e.g., in real time, for example based on tracking of the operations of one or more surgical tools.

In some embodiments, the surgeon may operate the MR device 200 to render a hologram of the prosthetic cup component/HipXpert tool/pelvis with the hologram of the cup component at the planned orientation and location within the acetabulum. The surgeon may operate user interface elements provided by the MR device 200 to resize, move, and/or rotate the hologram of the cup component/HipXpert tool/pelvis so that the hologram is co-located with the physical HipXpert tool attached to the patient's pelvis. The surgeon may operate the MR device 200 to peg or anchor the hologram of the cup component/HipXpert tool/pelvis at that location within the operation room. The surgeon may look through the incision in the patient and compare the location and orientation of the physical cup component with the hologram of the cup component. The surgeon may determine whether the appearance of the physical cup component as implanted matches the hologram of the cup component. If not, the surgeon may reposition the physical cup component until it matches the hologram of the cup component.

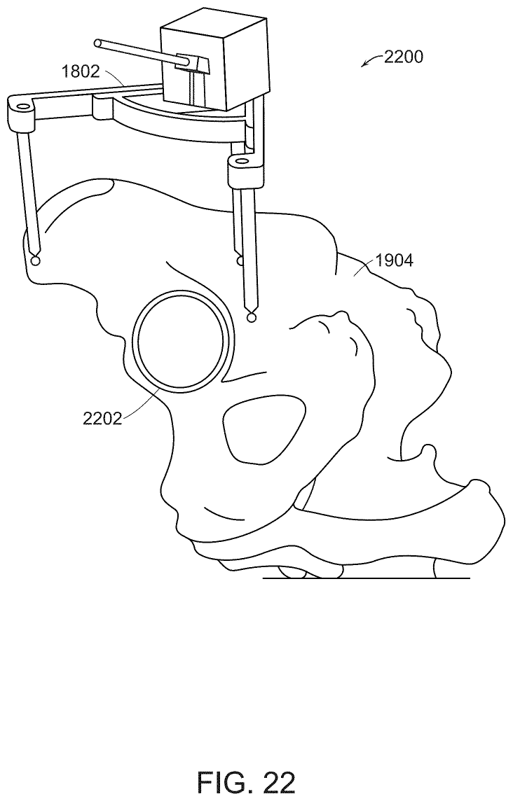

FIG. 22 is a pictorial representation of a hologram 2200 in accordance with one or more embodiments. The hologram 2200 may include the hologram 1802 of the HipXpert device, the hologram 1904 of the patient's pelvis, and a hologram 2202 of the cup component implanted in the patient's acetabulum as planned. During the surgical procedure, the hologram 2200 may be co-located to the corresponding physical objects either manually and/or automatically, for example by co-locating the hologram 1802 of the HipXpert device with the physical HipXpert device. The surgeon may then examine the physical cup component as implanted, e.g., through the use of the cup impactor, and see if the location and orientation of the physical cup component matches the hologram 2202 of the cup component as planned. If not, the surgeon may reposition the physical cup component, e.g., using the cup impactor, until the location of the physical cup component matches the hologram 2202.

In some embodiments, the surgeon may utilize the hologram 2200 to determine where to insert one or more screws for holding the physical cup component in place. More specifically, the surgeon may base his or her decision on where to place the one or more screws based on the hologram 1904 of the patient's pelvis. For example, the surgeon may place the one or more screws such that they are anchored securely to the patient's pelvis as indicated by the hologram 1904. For example, the cup may be planned such that the screw holes in the cup are optimally positioned to achieve the best fixation with the screws, and the surgeon may co-locate the physical cup with the hologram during surgery thereby implementing the planned best fixation.

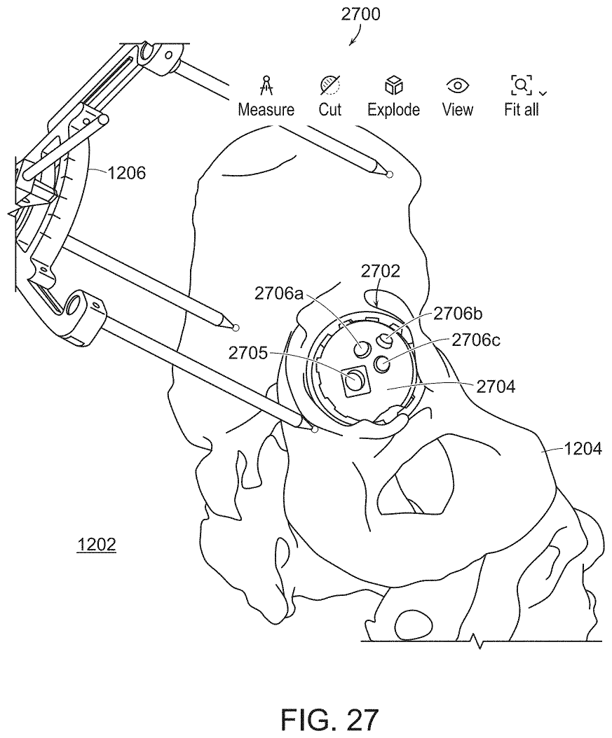

FIG. 27 is an illustration of an example planning window 2700 generated by the surgical planning system 1100 and presented on the display 1118 in accordance with one or more embodiments. The planning window 2700 includes a model pane 1202 presenting a 3D model of the patient's pelvis 1204. Docketed to the model of the pelvis 1204 is a 3D model of the HipXpert tool 1206. The pelvis 1204 includes an acetabulum 2702 and disposed in the acetabulum 2702 is a shell 2704 of an acetabular cup component. The shell 2704 includes a dome hole 2705 for attaching the shell 2704 to a cup impactor and three screw holes 2706a-c for receiving bone screws for securing the shell 2704 to the acetabulum 2702. The shell 2704 may be rotated within the acetabulum 2702 thereby changing where the screws enter the pelvis. The location of the shell 2704 may be planned so that the bone screws will penetrate bone, improving fixation of the screws to the pelvis. The position of the screw holes 2706a-c also may be planned so that the bone screws do not extend beyond the bone and injure a blood vessel or other object. Here, the shell 2704 is positioned at minus 20 degrees of rotation. In this location, the anterior/inferior screw inserted in the screw hole 2706c may have to be short and may even penetrate the anteromedial inner cortex, presenting risk to vital structures of the patient.

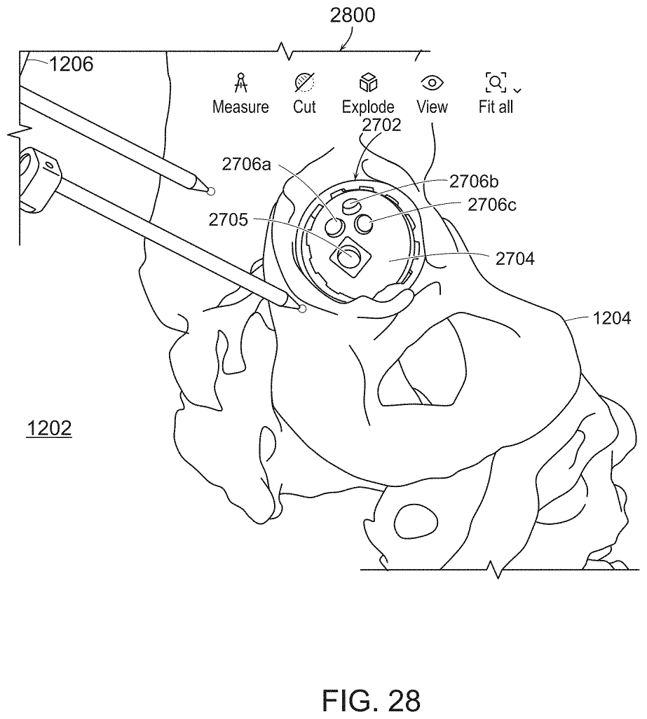

FIG. 28 is an illustration of an example planning window 2800 generated by the surgical planning system 1100 and presented on the display 1118 in accordance with one or more embodiments. The planning window 2800 includes a model pane 1202 presenting a 3D model of the patient's pelvis 1204 and the HipXpert device 1206. Here, the shell 2704 is moved to a new location in the acetabulum 2702 relative to the location illustrated in FIG. 27. Specifically, the shell 2704 is positioned at plus 20 degrees of rotation. In this location, the posterior inferior screw hole 2706b is getting closer to where it might need to have a short length to avoid extending beyond the posterior wall.

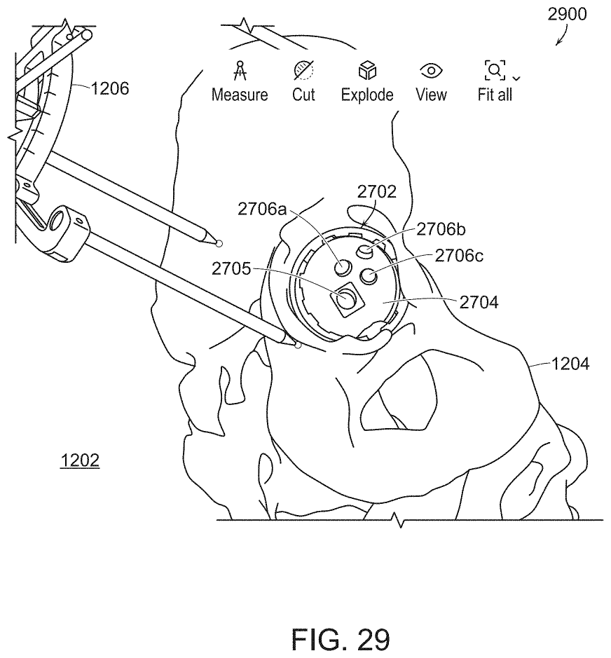

FIG. 29 is an illustration of an example planning window 2900 generated by the surgical planning system 1100 and presented on the display 1118 in accordance with one or more embodiments. The planning window 2900 includes a model pane 1202 presenting a 3D model of the patient's pelvis 1204 and the HipXpert device 1206. Here, the shell 2704 is moved to a new location in the acetabulum 2702 relative to the location illustrated in FIGS. 27 and 28. Specifically, the shell 2704 is positioned at zero degrees of rotation. At this location, all of the screw holes 2706a-d are in locations that provide excellent screw length supporting strong bone fixation. Accordingly, the planner may choose zero degrees of rotation for the planned location of the shell during surgery. Furthermore, one or more holograms may be generated based on the models of the hip, the HipXpert device, and the shell as illustrated in FIG. 29. The hologram may be presented during surgery and the surgeon may align the physical shell with the shell included in the hologram so that the screw holes are in the planned locations.

In some embodiments, in addition to determining ideal locations for the screw holes of the shell, the direction and lengths of the bone screws in the screw holes may also be planned. The direction of the bone screws may be planned to maximize screw fixation and/or avoid penetrating beyond the bone or causing any injury. One or more holograms may be generated that illustrate the planned directions and lengths of the bone screws. The representation of the direction of the bone screws may be illustrated in several ways. For example, a line showing the directions may be included in the holograms and the surgeon may operate a drill to drill holes for the bone screws along these lines. In other embodiments, holograms of the bone screws at the planned directions with the tips at the screw holes may be provided. It should be understood that the planned directions of the bone screws may be illustrated in the hologram in other ways.

In some embodiments, the drilling depth for the bone screws and/or the size, e.g., length, of each bone screw may be presented in one or more holograms. For example, a hologram of a drill at the planned depth and with the drill bit in the planned direction may be presented. The surgeon may operate a physical drill so that the physical drill bit is in the planned direction and the surgeon may stop drilling when the physical drill reaches alignment with the hologram.

This approach for planning bone screws has several advantages. For example, it may reduce risk by avoiding dangerous drill trajectories, drilling too far, which might penetrate the far cortex in a dangerous location, reduce the risk of placing a screw that is too long in the wrong place, reduce risk by avoiding short screws when longer screws can be safely placed, and save time since the surgeon need not measure screw depths during the surgical procedure. It also avoids the risk of using screws that are unnecessarily short that would have poor purchase.

Planning Phase

A surgeon may interact with the surgical planning system 1100 to plan where to position an acetabular cup component and thus the trajectories of the screws used to secure the cup to the patient's pelvis. The surgeon may also interact with the surgical planning system 1100 to choose screws that are as long as possible while not extending beyond the pelvis and into tissue.

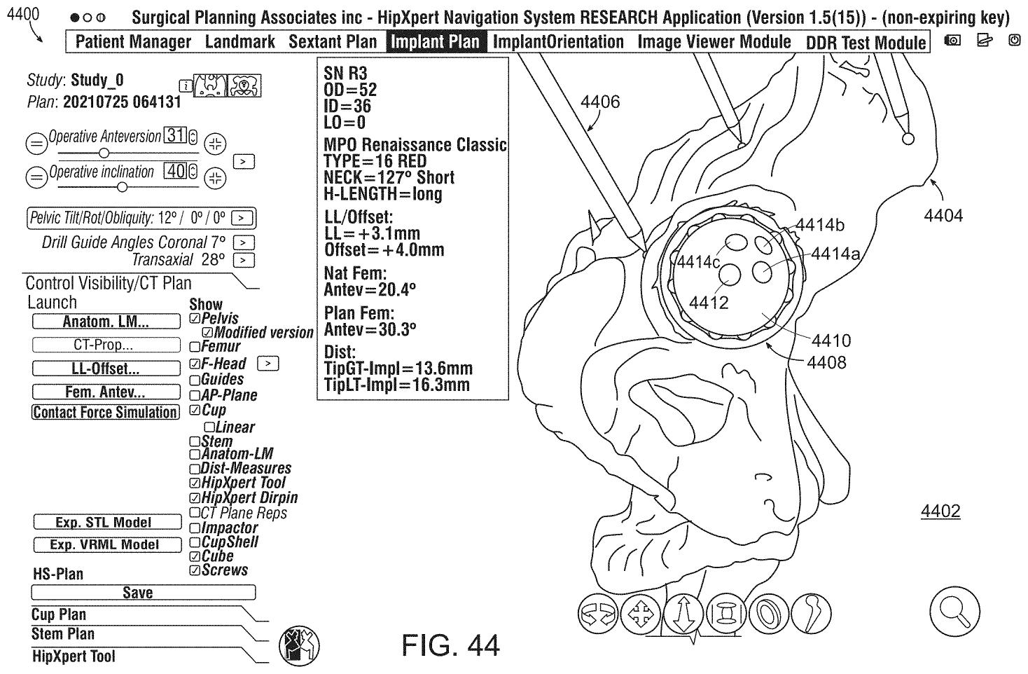

FIG. 44 is an illustration of an example planning window 4400 that may be generated by the surgical planning system 1100 and presented on the display 1118 in accordance with one or more embodiments. The planning window 4400 may include a model pane 4402 presenting a 3D model 4404 of a patient's pelvis. Docketed to the model 4404 of the pelvis may be a 3D model 4406 of a registration tool of which only the legs are visible. The pelvis model 4404 includes an acetabulum 4408 and disposed in the acetabulum 4408 is a 3D model 4410 of a shell of an acetabular cup component. As noted, the pelvis model 4406 may be generated from patient specific data, such as one or more CT or other imaging studies of the patient's pelvis. Accordingly, the pelvis model 4406 may accurately represent the shape and dimensions of the patient's pelvis. The 3D shell model 4410 may be generated from one or more CAD or other files for the shell. Thus, the shell model 4410 may accurately represent the shape and dimensions of a physical shell selected by the surgeon for the surgical procedure. The surgical planning system 1100 may include a library of 3D models and/or CAD files of available cups. A surgical planner, who may or may not be a surgeon, may select the 3D model and/or CAD files associated with the shell of the cup selected for the patient. The 3D model or CAD file includes the size and location of the shell's screw holes. In some embodiments, the planning tool 1106 may resize the 3D shell model 4410 to match the scale of the pelvis model 4404.

The shell 4410 includes a threaded dome hole 4412 for attaching the shell 4410 to a cup impactor. The shell 4410 also includes three screw holes 4414a-c for receiving bone screws for securing the shell 4410 to the patient's pelvis within the acetabulum. The shell 4410 may have a central axis extending through the dome hole 4412. A surgeon planning the surgery may plan an orientation of the central axis within the native acetabulum 4408, e.g., by interacting with the planning window 4400. The determination of an orientation may be based on many factors. As noted, orientation of the shell 4410, and thus by extension orientation of the cup component, may refer to the angle of the cup's central axis relative to a coordinate frame of the pelvis, such as the Anterior Pelvic Plane (AP Plane). For example, a surgeon may start with a default orientation of 29 degrees of operative anteversion and 40 degrees of operative inclination relative to the AP Plane. For example, these values may be determined through research to minimize the post-surgery dislocation rate. The surgeon may then modify that default orientation on a patient specific basis based on spine-pelvis considerations, such as supine pelvic tilt measurement, standing pelvic tilt measure, or dynamic assessment of the spine-pelvis region. For example, for a patient with less pelvic tilt than normal, which effectively decreases the front coverage of the cup, the surgeon may decrease the operative anteversion to compensate for the lower pelvic tilt. Alternatively, if a patient has more pelvic tilt than normal, the surgeon may increase the operative anteversion. In other embodiments, cup orientation may be refined based on simulated activities and associated potential edge loading of forces on the edge of the cup component, range of motion simulation, and/or calculation of bone-bone, bone-implant, or implant-implant impingement.

The surgeon may also plan an x,y,z, position of the shell 4410 within the acetabulum 4408. Like the determination of the shell's orientation, the determination of the shell's x,y,z, position may take into consideration the factors described above, but may not take optimal screw fixation and optimal screw length into consideration.

Once the orientation and x,y,z position components of the shell's placement within the acetabulum have been determined, the surgical planner may rotate the shell 4410 within the acetabulum 4408 about its central axis, again by interacting with the planning window 4400. By rotating the shell 4410 within the acetabulum, the surgical planning system 1100, which has access to the 3D models and/or CAD files of the shell, changes the locations of the screw holes and thus the points at which the screws extending through the shell enter the patient's pelvis. Nonetheless, rotation of the shell 4410 may not alter the planned orientation and x,y,z position of the shell 4410. During the planning stage, the surgeon can interact with the surgical planning system 1100 to rotate the shell 4410 and evaluate how different rotational positions of the shell 4410 affect potential screw lengths. The surgeon may select a final rotational position of the shell model 4410 such that physical screws of desired lengths, such as screws of the longest possible lengths, can be used to secure the physical shell to the patient's pelvis during surgery. In some embodiments, additional factors beyond screw length may be considered when determining the final rotational position of the shell model 4410. Other exemplary factors include patient safety, e.g., regarding the direction chosen for the screws, and the quality of the bone to be purchased by the screws. For example, to avoid perforating the external iliac artery, which passes close to the medial wall of the acetabulum anteriorly, the surgeon may avoid placing a screw that passes through the far cortex, which might perforate the far cortex. Conversely, there are other locations where passing the drill through the far cortex is safe and so deliberately placing a screw that captures the far cortex would facilitate much better fixation. So, in addition to screw length, which may be a primary consideration, deliberately catching the far cortex in a safe way or deliberately planning the screws to be placed in “better” bone, such as stronger bone or avoiding bone cysts, are other optimizations the surgeon may consider. Screw length, patient safety and bone quality may also be used by the surgeon in choosing a particular cup to use during surgery. For example, different cups have screw holes in different locations or different numbers of screw holes.

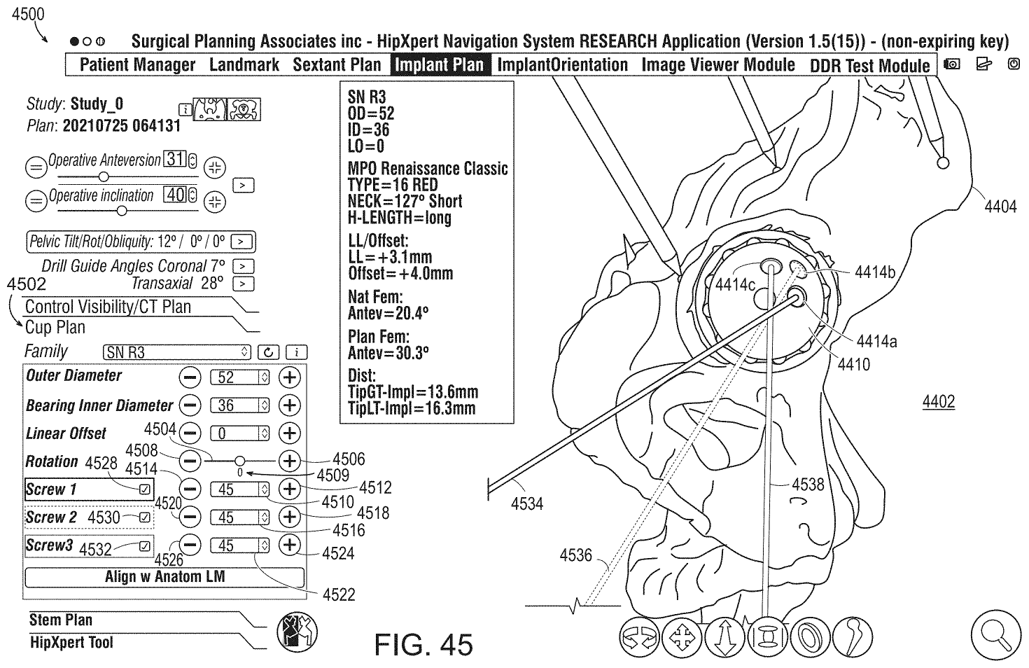

FIG. 45 is an illustration of an example planning window 4500 that may be generated by the surgical planning system 1100 and presented on the display 1118 in accordance with one or more embodiments. The planning window 4500 may include the model pane 4402 presenting the pelvis model 4404 and the shell model 4410. The planning window 4500 also may include a cup plan window 4502 having window elements, such as widgets or other interface elements, for receiving information from and/or presenting information to a surgeon planning the surgery. For example, the cup plan window 4500 may include one or more input elements for entering a rotation value for the shell 4410 within the acetabulum, such as a slider 4504 and plus and minus buttons 4506 and 4508. The cup plan window 4500 also may include a numeric display element 4509 that presents the current value of the rotation of the shell 4410 within the acetabulum. The cup plan window 4500 also may include one or more input elements for specifying the lengths of the three screws used to secure the shell 4410. For example, for each screw corresponding to one of the screw holes 4414a-c, the cup plan window 4500 may include a numeric entry box and plus and minus buttons, which may be used to specify a respective screw length. For the screw hole 4414a, the window 4500 may include numeric entry box 4510 and plus and minus buttons 4512 and 4514. For the screw hole 4414b, the window 4500 may include numeric entry box 4516 and plus and minus buttons 4518 and 4520. For the screw hole 4414c, the window 4500 may include numeric entry box 4522 and plus and minus buttons 4524 and 4526.

For each screw, the cup plan window 4500 also may include an input element, such as check boxes 4528, 4530 and 4532, to toggle a display of the trajectory of the respective screw. In response to the check box being marked for a given screw, the planning tool 1106 may present a display element such as a line representing the trajectory of the given screw as installed in the respective screw hole 4414a-c. As each of the checkboxes 4528, 4530, and 4532 shown in FIG. 45 is marked, e.g., checked, the planning tool 1106 presents trajectories 4534, 4536, and 4538 for the respective screws in the model pane 4402 of the planning window 4500. The planning tool 1106 may present the trajectories 4534, 4536 and 4538 as extending outwardly relative to the inside of the shell 4410. That is, the trajectories 4534, 4536 and 4538 may represent the paths to be followed by the screws during their installation through the respective screw holes 4414a-c and into the pelvis model 4404. In some embodiments, the planning tool 1106 may present the trajectories 4534, 4536 and 4538 based on the screws being installed along center lines of the respective screw holes 4414a-c. That is, the trajectories 4534, 4536 and 4538 may be centered on the respective screw holes 4414a-c. Nonetheless, in other embodiments, the planning tool 1106 may present the trajectories 4534, 4536 and 4538 at one or more offset angles from the center lines of the screw holes 4414a-c.

In some embodiments, the screw trajectories 4534, 4536 and 4538 may be presented as colored lines, where each screw trajectory has a different color to aid in distinguishing the screw trajectories 4534, 4536 and 4538 from each other. Nonetheless, it should be understood that other display elements or graphical affordances besides color coded lines may be used by the planning tool 1106, such as dashed lines, animated lines, drill bits, screws, etc.

In addition to presenting the trajectories 4534, 4536 and 4538 of the screws as extending out from the inside of the shell model 4410 away from the pelvis model 4404, the planning tool 1106 also may present display elements representing the screws themselves as fully installed through the respective screw holes 4414a-c and into the pelvis, based on the locations of the screw holes 4414a-c within the acetabulum and the lengths of the respective screws. For example, the planning tool 1106 may utilize the location of the shell model 4410 in the acetabulum 4408 including the shell's rotation within the acetabulum 4408, the surface shape and/or geometry of the pelvis as represented by the pelvis model 4404, and the geometry, e.g., length, of the screws, so that the screw elements displayed in the model pane 4402 accurately model the physical screws installed at the respective screw holes 4414a-c.

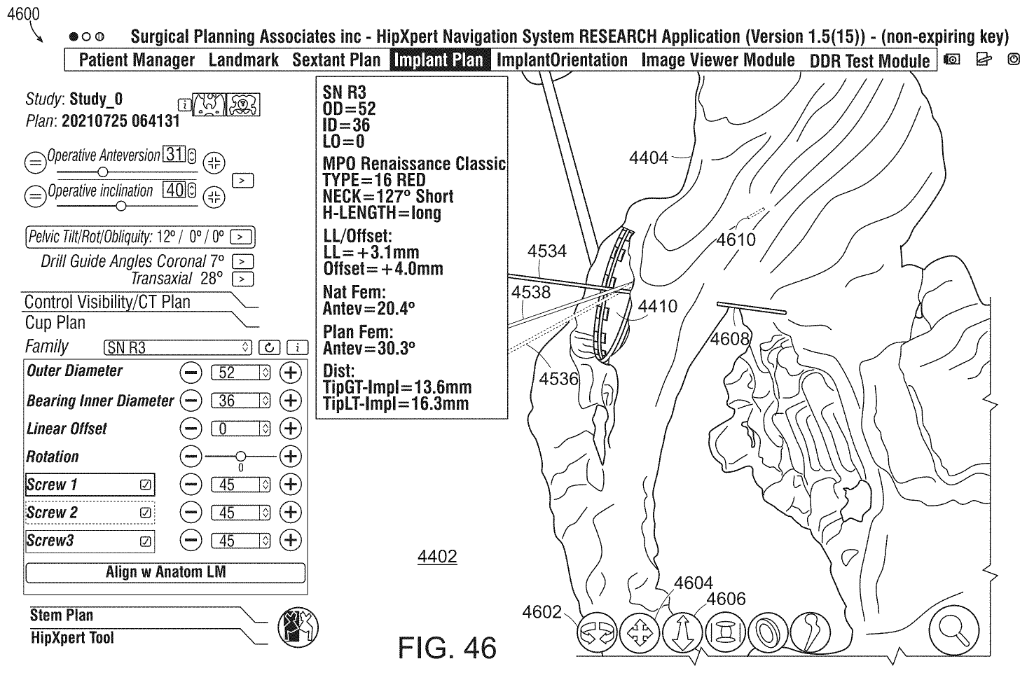

FIG. 46 is an illustration of an example planning window 4600 that may be generated by the surgical planning system 1100 and presented on the display 1118 in accordance with one or more embodiments. The planning window 4600 may include the model pane 4402 presenting the pelvis model 4404, the shell model 4410, and the three screw trajectories 4534, 4536 and 4538. In FIG. 46, the pelvis model 4404 is rotated relative to the position of the pelvis model illustrated in FIGS. 44 and 45 in order to show the back side of the acetabulum, e.g., the interior of the pelvis. That is, in FIGS. 44 and 45, the pelvis model 4404 is rotated so as to provide a top view of the acetabulum, while in FIG. 46, the pelvis model 4404 is rotated to provide a side view of the acetabulum. The surgeon may interact with the planning window 4600 to position the pelvis model 4404 it as desired within the model pane 4402. For example, the planning tool 1106 may provide command buttons, such as buttons 4602, 4604 and 4606, on the planning window 4600 for rotating, zooming, and sliding the pelvis model 4404 on the model pane 4402. The shell model 4410 may be fixed within the acetabulum of the pelvis model 4404 so that the shell model 4410 rotates along with the pelvis model 4404. Similarly, the trajectories 4534, 4536 and 4538 may be fixed within the respective screw holes 4414a-c so that the trajectories 4534, 4536 and 4538 also rotate along with the pelvis model 4404.

The planning tool 1106 may also use an initial or default screw length, such as 45 millimeters (mm), and an initial or default rotation value for the shell model 4410, such as zero degrees rotation. In some embodiments, the planning tool 1106 may only show those portions, if any, of the display elements representing the screws as installed in the screw holes 4414a-c that extend beyond the surface of the pelvis model 4404. In some embodiments, the same display elements used for the trajectories 4534, 4536 and 4538, e.g., color coded lines, may be used as the display elements for the screws. Nonetheless, it should be understood that other display elements or graphical affordances may be used for the screws. For example, bone screw models based on CAD files for the bone screws may be used as the display elements. In other embodiments, dashed lines, animations, etc. may be used as the display elements for the screws as installed at the screw holes 4414a-c.

As illustrated in FIG. 46, a portion of the screw installed at the screw hole 4414a extends beyond the surface of the patient's pelvis, as indicated by screw element portion 4608. The remaining portion of this screw, which is within the pelvis model 4404 is not shown. Similarly, a portion of the screw installed at the screw hole 4414b also extends beyond the surface of the patient's pelvis, as indicated by screw element portion 4610. Again, the remaining portion of this screw, which is within the pelvis model 4414 is not shown. The screw element portions 4608 and 4610, as presented by the planning tool 1106, indicate how much of the respective screws would extend beyond the surface of the pelvis and into patient tissue. That is, the planning tool 1106 may scale the lengths of the display elements representing the screws are scaled to be in proportion to the pelvis model 4404 presented on the model pane 4402.

As illustrated in FIG. 46, a larger portion of the screw installed at screw hole 4414a extends beyond the surface of the pelvis model 4404 opposite the acetabulum, as compared to the portion of the screw installed at screw hole 4414b that extends beyond the surface of the pelvis model 4404. With reference to FIGS. 45 and 46, no portion of the screw installed at the screw hole 4414c is shown extending through the surface of the pelvis model 4404. Accordingly, this screw is completely within the pelvis. The surgeon may alter the orientation, e.g., rotation, of the shell model 4410 within the acetabulum and/or change, e.g., shorten, the lengths of the screws installed at screw holes 4414a and 4414b so that these two screws also remain completely within the pelvis model 4404.

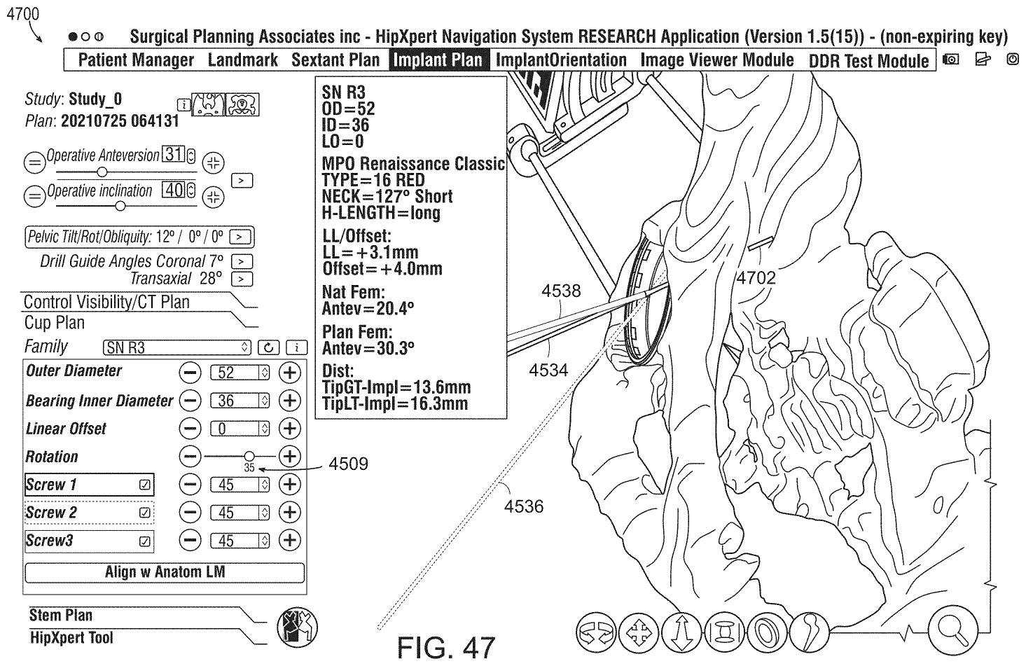

FIG. 47 is an illustration of an example planning window 4700 that may be generated by the surgical planning system 1100 and presented on the display 1118 in accordance with one or more embodiments. As indicated by the value for the slider 4504, i.e., ‘35’, in FIG. 47, the shell model 4410 has been rotated ‘35’ degrees as indicated at numeric display element 4509 relative to the orientation of the shell 4410 as illustrated in FIGS. 45 and 46, in which the shell was at ‘0’ degrees orientation. In FIG. 47, there is no longer any portion of the screw installed in the screw hole 4414b shown as extending beyond the surface of the pelvis model 4404. Accordingly, by rotating the shell model 4410 to 35 degrees, the screw installed at the screw hole 4414b is now completely within the pelvis. However, a portion of the screw installed at the screw hole 4414a, as indicated at 4702, is still shown as extending beyond the surface of the pelvis. The portion 4702 is smaller than the portion 4608 (FIG. 46) indicating that more of the screw installed at the screw hole 4414a is within the pelvis. Nonetheless, a portion of this screw still extends beyond the surface of the pelvis as indicated by the screw display element portion 4702.

In order to get the screw installed at the screw hole 4414a to be completely within the pelvis model 4404 the surgeon may further change the rotation of the shell model 4410 within the acetabulum and/or change the length of the screw used at the screw hole 4414a. Suppose the surgeon changes the length of the screw used at the screw hole 4414a from 40 mm to 35 mm.

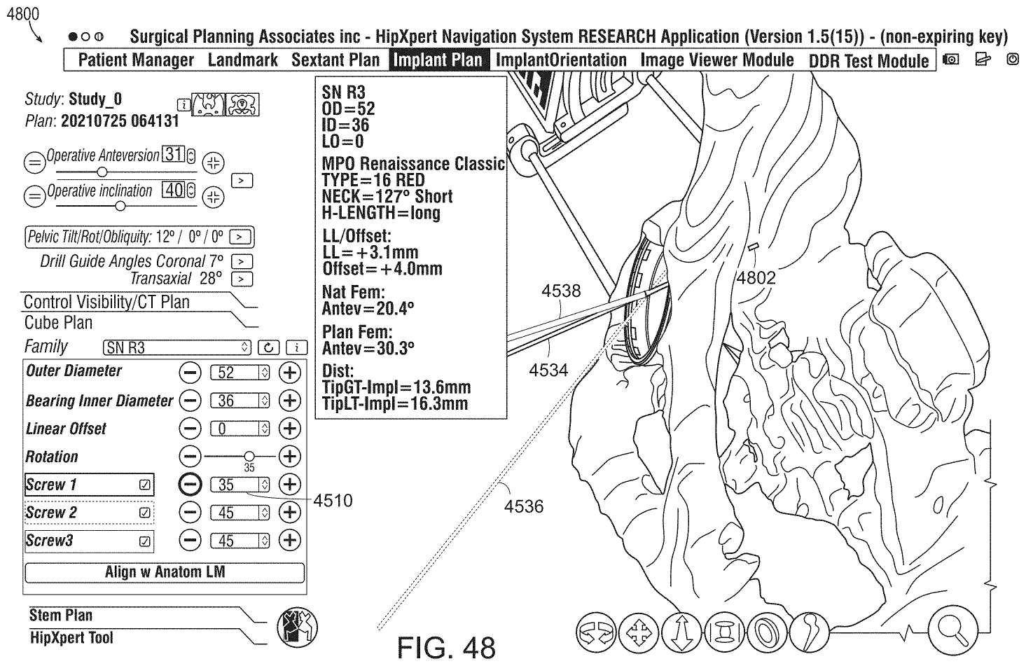

FIG. 48 is an illustration of an example planning window 4800 that may be generated by the surgical planning system 1100 and presented on the display 1118 in accordance with one or more embodiments. As shown in the numeric entry box 4510, the surgeon has changed the length of the screw used at the screw hole 4414a to 35 mm. Nevertheless, a portion of the screw installed at the screw hole 4414a, as indicated at display element portion 4802, is still shown as extending beyond the surface of the pelvis. The portion 4802 is smaller than even the portion 4702 (FIG. 47) indicating that even more of the screw installed at the screw hole 4414a is within the pelvis. Nonetheless, a portion of this screw still extends beyond the surface of the pelvis, as indicated by the screw display element portion 4802. The surgeon may further shorten the length of the screw used at the screw hole 4414a.

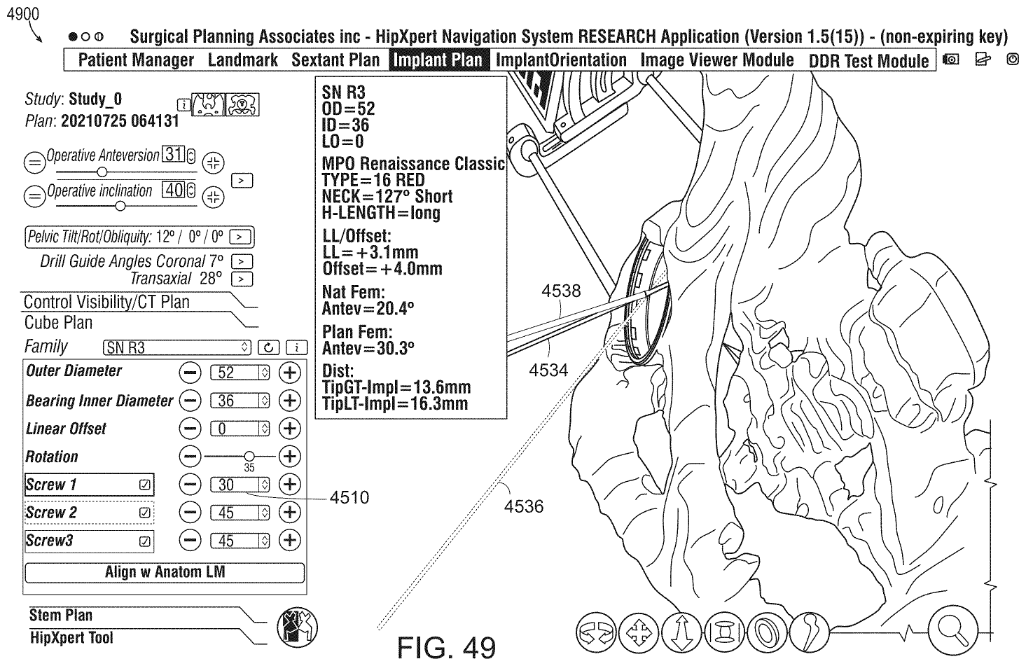

FIG. 49 is an illustration of an example planning window 4800 that may be generated by the surgical planning system 1100 and presented on the display 1118 in accordance with one or more embodiments. As shown in the numeric entry box 4510, the surgeon has changed the length of the screw used at the screw hole 4414a to 30 mm. Now, there is no longer any portion of the screw installed at the screw 4414a shown extending through the surface of the pelvis model 4404. Accordingly, by selecting a screw length of 30 mm the surgeon is able to choose a screw that will not extend through the surface of the pelvis and into the patient's tissue.

In some embodiments, the surgical planner may start with all three screws having relatively long lengths for use in securing a shell, such as 45 mm. The surgeon may then rotate the cup so that the screws in the middle and in the back, which as noted above are quite long, e.g., 45 mm, are completely within the bone of the pelvis. The surgeon may then rotate the view around to see the anterior screw trajectory plan, and may reduce its length until it to is completely within the bone. In some cases, this may result in a screw length of 20 mm or 15 mm, which may be the shortest option available.

In some embodiments, the surgical planner may vary the angle of the screws through the screw holes relative to the perpendicular through the screw holes. For example, the planning window may provide an optional screw angle, such as six degrees. So the surgical planner could consider increased complexity to achieve even longer screws if he or she angled one screw 6 degrees one way relative to perpendicular to the screw hole and angled another screw 6 degrees another way, for example. That is, instead of being a perpendicular ray, the screw trajectory planning opportunity may be a cone with the apex of the cone being at the location of the screw hole, and the cone angle being twelve degrees for example, nonetheless other cone angles may be utilized such as fifteen degrees.

As shown, by determining and presenting screw trajectory display elements and screw display elements, the planning tool 1106 allows a surgeon to evaluate any number of combinations of shell rotational positions and screw lengths to arrive at desired values, e.g., values for shell rotation and screw lengths that maximize the screw lengths while ensuring none of the screws will extend beyond the surface of the pelvis. Once values for the rotation of the shell model 4410 within the acetabulum 4408 and for the screw lengths have been determined so that the screws are considered long enough to secure the shell to the patient's pelvis while not extending beyond the surface of the pelvis, the planned trajectories and screw lengths may be saved in the surgical plan 1114.

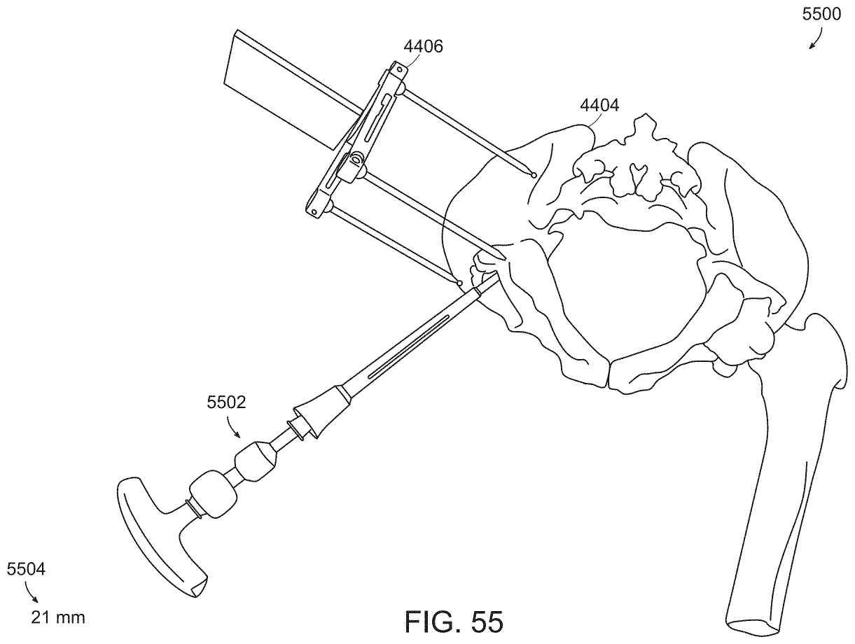

FIG. 55 is an illustration of an example planning window 5500 that may be generated by the surgical planning system 1100 and presented on the display 1118 in accordance with one or more embodiments. The planning window 5500 may include the 3D registration tool model 4406 docked to the 3D pelvis model 4404. The planning window 5500 may also include a 3D model of screwdriver 5502 in a planned position and at a planned angle for implanting one of the bone screws. The 3D model of the screwdriver 5502 may be generated from one or more CAD files for a physical screwdriver to be used during the surgical procedure. The planning window 5500 may also present information on the bone screw associated with the screwdriver model 5502. For example, the planning window 5500 may present the length, e.g., 21 mm, of the planned bone screw.

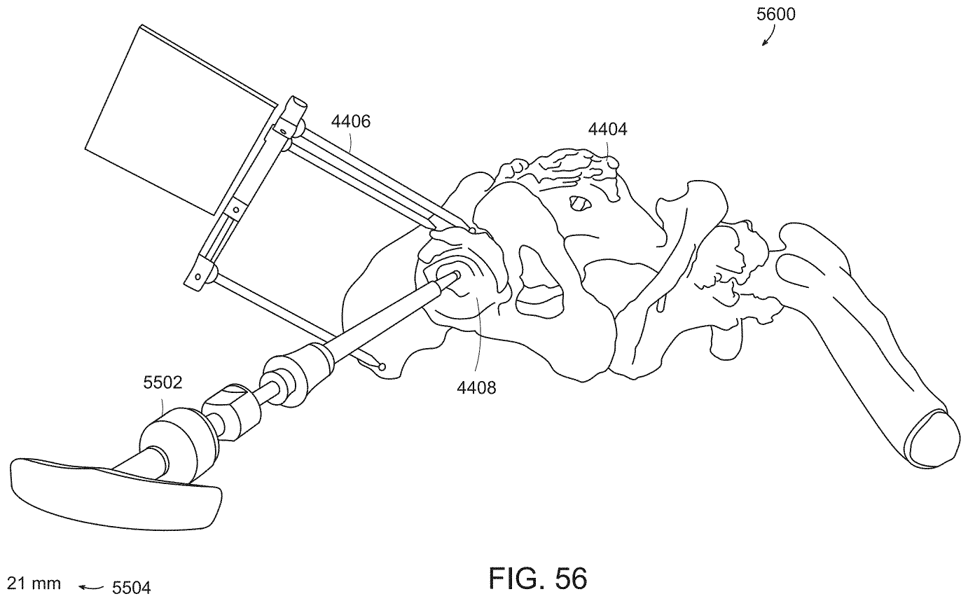

FIG. 56 is an illustration of an example planning window 5600 that may be generated by the surgical planning system 1100 and presented on the display 1118 in accordance with one or more embodiments. The planning window 5600 may present similar information as the planning window 5500 (FIG. 55). However, the 3D pelvis model 4404 may be rotated from the position shown in FIG. 55 so as to view the acetabulum.

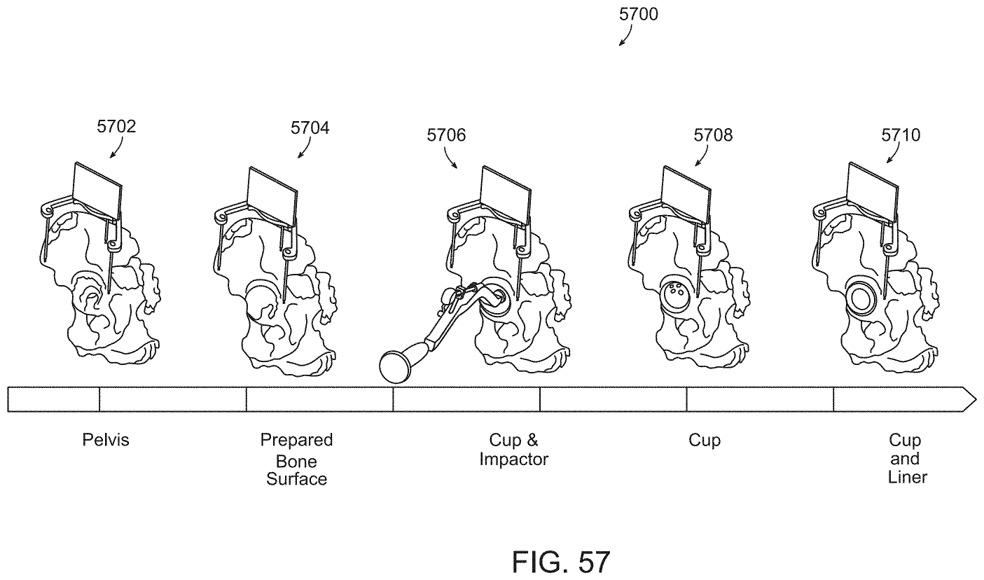

FIG. 57 is an illustration of an example sequence of images 5700 representing at least part of a surgical plan in accordance with one or more embodiments. The sequence 5700 may include a first image 5702 presenting the 3D registration tool model 4406 as docked to the 3D pelvis model 4404 in the planned manner, a second image 5704 presenting a prepared bone surface of the acetabulum, a third image 5706 of the cup in the planned position and orientation in the acetabulum and a cup impactor at a planned position and angle to implant the cup as planned, a fourth image 5708 of the cup in the planned position and orientation in the acetabulum, and a fifth image 5710 of the cup and liner in the planned position and orientation in the acetabulum.

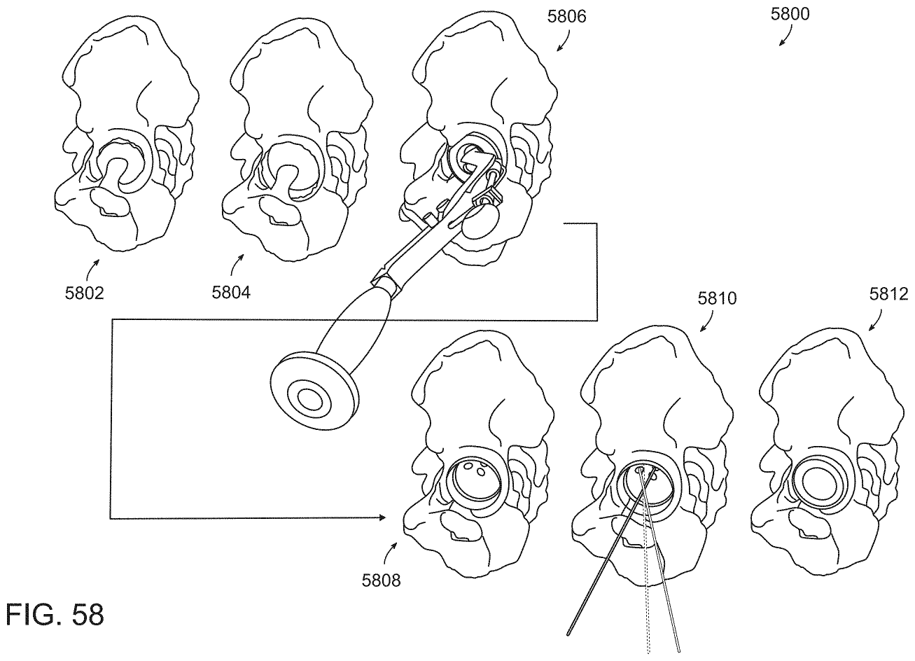

FIG. 58 is an illustration of an example sequence of images 5800 representing at least part of a surgical plan in accordance with one or more embodiments. The sequence 5800 may include a first image 5802 presenting the 3D pelvis model 4404, a second image 5804 presenting a prepared bone surface of the acetabulum, a third image 5806 of the cup in the planned position and orientation in the acetabulum and a cup impactor at a planned position and angle to implant the cup as planned, a fourth image 5808 of the shell in the planned position and orientation in the acetabulum, a fifth image 5810 of the shell and the planned screw trajectories, and a sixth image 5812 of the cup and liner in the planned position and orientation in the acetabulum.

One or more holograms may be generated based on information included in the surgical plan 1114. For example, one or more holograms may be generated showing the planned placement of the physical shell in the acetabulum. One or more holograms also may be generated showing the screw trajectories as planned. The holograms may include holograms for the pelvis model 4404, the shell model 4410, and the display elements representing the trajectories 4534, 4536 and 4538. In some embodiments, the planning tool 1106 may export at least some of these 3D model files into a format compatible with the MR device 200 so that the MR device 200 may project holograms corresponding to the exported 3D model files. For example, one or more of the files representing the 3D objects may be exported and loaded into the memory of the MR device 200. Alternatively, the files representing the 3D objects may be stored at a server and the MR device 200 may be configured as a client capable of accessing those files from the server. In some embodiments, the MR device 200 may be a see-through head mounted display (HMD).

In some embodiments, a method of planning screw lengths and trajectories, such as for a 3-hole acetabular cup, may include:
starting with extra-long screws for all three holes, e.g., 45 mm;
rotating the cup, once the cup is in the planned position and orientation, so that the two posterior screws are fully within the bone; and
reducing the size of the third screw, which will initially be protruding beyond the bone, so that it is within the bone.
   In some cases, the last step may include rotating the 3D model of the pelvis to provide a better view of the third screw.

Surgical Phase

During the surgical procedure, one or more registration and tracking devices may be docked to the patient in a planned location. In some embodiments, the registration and tracking device may include a target plate and one or more two-dimensional (2D) codes may be placed on the front and/or back faces of the target plate. For example, one 2D code may be disposed on the front face, while another 2D code may be disposed on the back face. Each 2D code may define a coordinate system. The coordinate system may have a known relationship to a coordinate system for the patient's anatomy when the registration and tracking device is docked to the patient's anatomy as planned, such as the AP Plane. The MR device 200 may detect at least one of the 2D codes and utilize the coordinate system for the detected 2D code and a transformation matrix to present the one or more holograms in predetermined positions relative to the patient's anatomy, such as the AP Plane. In addition to detecting the one or more 2D codes, the MR device may track the 2D code, e.g., as the surgeon wearing the MR device moves his or her head. For example, the MR device may anchor the one or more holograms in space based on the spatial coordinate system exposed by and aligned with the at least one detected 2D code. The holograms may show the planned locations of the surgical tools, implants, and devices, as planned for the surgical procedure, and the surgeon may align physical tools, implants, and devices with the holograms.

By virtue of the unique registration and tracking device, which is docked to the patient's anatomy in a planned manner and presents the one or more 2D codes, the present disclosure can register the pelvis without having to attach a tracker to the patient's pelvis and digitize numerous points on the pelvis.

Suitable MR devices for use with the present disclosure include the HoloLens series of mixed reality devices from Microsoft Corp., the Magic Leap One device from Magic Leap, Inc. of Plantation, FL, and the Blade smart glasses from Vuzix Corp. of West Henrietta, NY, among others, and are described in U.S. Patent Publication No. 2019/0025587 for MR Glasses with Event and User Action Control of External Applications to Microsoft Corp. and U.S. Patent Publication No. 2019/0285897 for Display Device to Apple Inc.

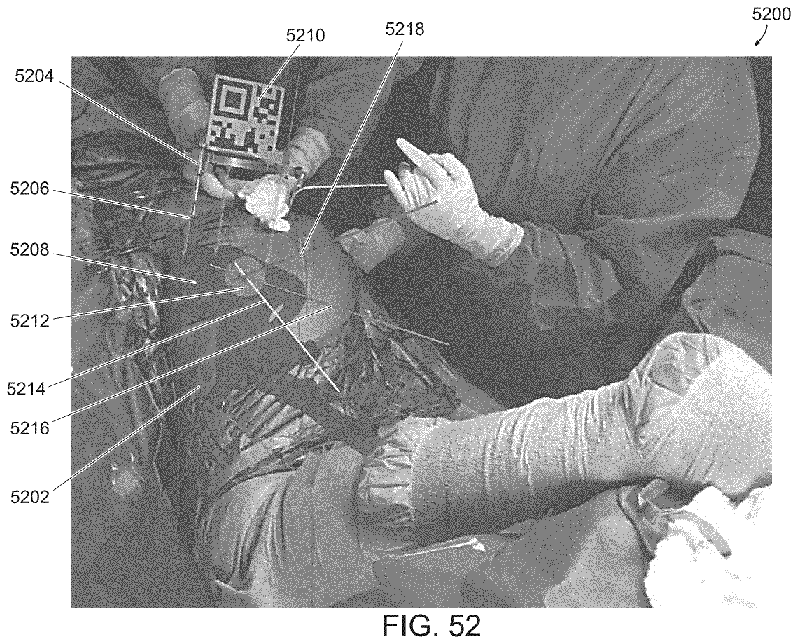

FIG. 52 is a pictorial representation of a surgical scene 5200 as viewed by a surgeon through an MR device, such as a see-through HMD, in accordance with one or more embodiments. The surgical scene 5200 includes physical elements that are visible to surgeon through the see-through HMD and holograms presented by the see-through HMD. For example, a physical patient's hip 5202 on which a surgical procedure, such as total hip arthroplasty, is being performed is visible through the see-through HMD. A physical registration and tracking device 5204 may be docked to the patient's pelvis. The see-through HMD presents a plurality of holograms which may be organized as a composite hologram. For example, the composite hologram may include a hologram 5206 of the registration and tracking device as docked to a hologram 5208 of the patient's pelvis as planned. The hologram 5208 of the pelvis is aligned with the patient's physical pelvis which is below the patient's skin and not visible to the surgeon wearing the see-through HMD. The see-through HMD may also present a hologram 5210 of a target plate with a two-dimensional (2D) code mounted on the hologram 5206 of the registration and tracking device. The see-through HMD may also present a hologram 5212 of an acetabular shell implanted in the acetabulum of the patient's pelvis 5208, as planned, and holograms 5214, 5216 and 5218 of screw trajectories for the screws installed at the screw holes of the shell, as planned. The surgeon may rotate the physical acetabular shell within the patient's acetabulum until physical acetabular shell is oriented as shown in the hologram 5212, thus achieving the planned placement of the physical acetabular shell in the acetabulum. For example, the surgeon may rotate the physical acetabular shell until the screw holes of the physical shell are aligned with the screw holes shown on the hologram 5212 of the acetabular shell. With the physical acetabular shell positioned as planned within the acetabulum, the surgeon may install physical bone screws to fixedly hold the physical acetabular shell in place. The surgeon may use physical bone screws having lengths as determined during the planning stage to ensure that none of the physical bone screws will extend beyond the surface of the patient's pelvis.

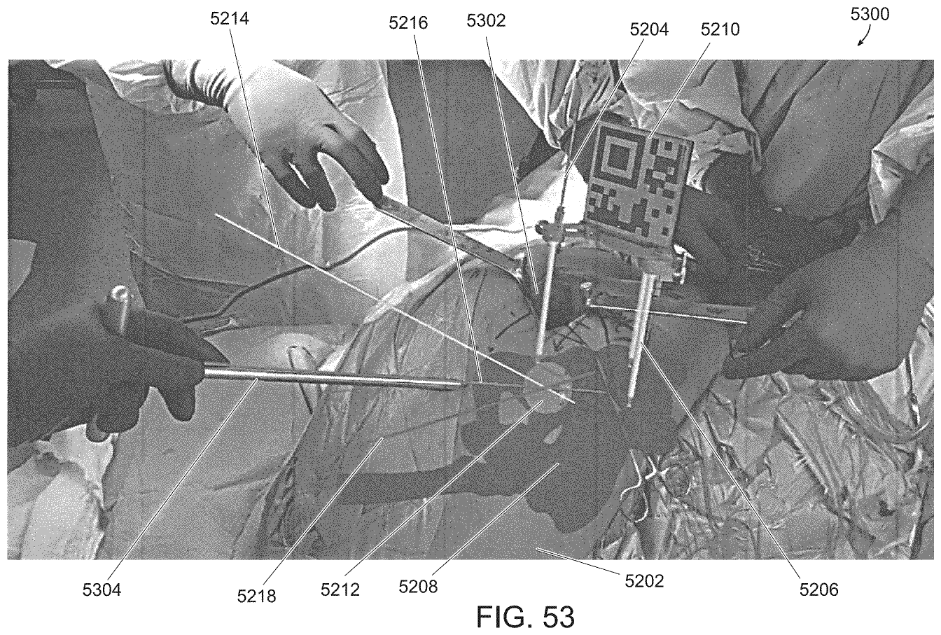

FIG. 53 is another pictorial representation of a surgical scene 5300 as viewed by a surgeon through an MR device, such as a see-through HMD, in accordance with one or more embodiments. The surgical scene 5300 includes the hologram 5206 of the registration and tracking device as docked to a hologram 5208 of the patient's pelvis. The scene 5300 also includes the acetabular shell hologram 5212 and the screw trajectory holograms 5214, 5216 and 5218. An incision 5302 through the patient's skin and tissue providing access to the patient's hip joint is also visible through the see-through HMD. The surgeon wearing the see-through HMD may operate a physical T-handle surgical screwdriver 5304 to implant physical bone screws as planned. For example, the surgeon, while wearing the see-through HMD, may align the longitudinal axis of the screwdriver 5304 with one of the screw trajectories, e.g., the screw trajectory 5216. The surgeon may keep the screw driver 5304 aligned with the respective screw trajectory hologram 5216 while implanting the respective bone screw to ensure that the bone screw is implanted along the planned trajectory.

It should be understood that the MR device may present other and/or additional holograms for the planned screw trajectories besides the colored lines. In some embodiments, the MR device may present a hologram of the screwdriver itself positioned according to the planned angle and depth of the screw. The MR device may present the hologram of the screwdriver alone, together with the colored line indicating the trajectory of the screw, or with one or more other holograms. A surgeon can then bring a physical screwdriver into the surgical scene. The surgeon can see both the physical screwdriver, at least part of which is outside of the patient's body, and the hologram of the screwdriver through the see-through HMD. The surgeon can utilize the screwdriver to implant the screw at the planned screw angle and depth by implanting the screw until the physical screwdriver is aligned with the hologram of the screwdriver in both angle and depth.

In some embodiments, the see-through HMD may present information associated with one or more of the screw trajectories. For example, the see-through HMD may present information such as screw length, screw size number, screw manufacturer, and/or screw model number.

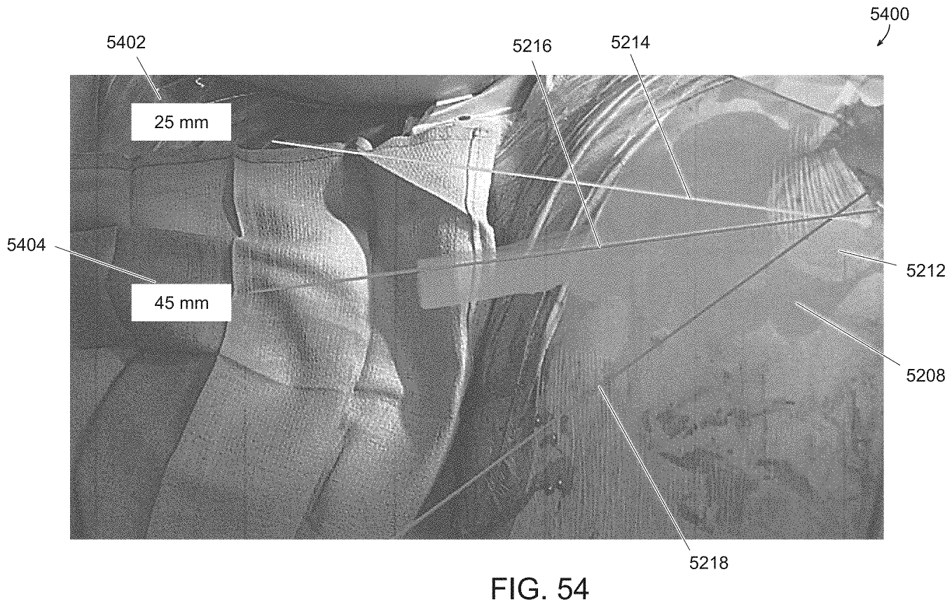

FIG. 54 is yet another a pictorial representation of a surgical scene 5400 as viewed by a surgeon through an MR device, such as a see-through HMD, in accordance with one or more embodiments. The surgical scene 5400 includes the hologram 5206 of the registration and tracking device as docked to a hologram 5208 of the patient's pelvis. The scene 5400 also includes the acetabular shell hologram 5212 and the screw trajectory holograms 5214, 5216 and 5218. The see-through HMD also presents information associated with at least some of the screw trajectory holograms 5214, 5216 and 5218. For example, the see-through HMD presents the length of bone screw associated with screw trajectory holograms 5214 and 5216. In particular, the see-through HMD presents length information 5402, e.g., 25 mm, associated with the screw trajectory hologram 5214. The see-through HMD also presents length information 5404, e.g., 45 mm, associated with the screw trajectory hologram 5216. Although not shown in FIG. 54, length information may also be provided for the screw trajectory hologram 5218. In some embodiments, the length information may be included as part of the holograms of the screw trajectories.

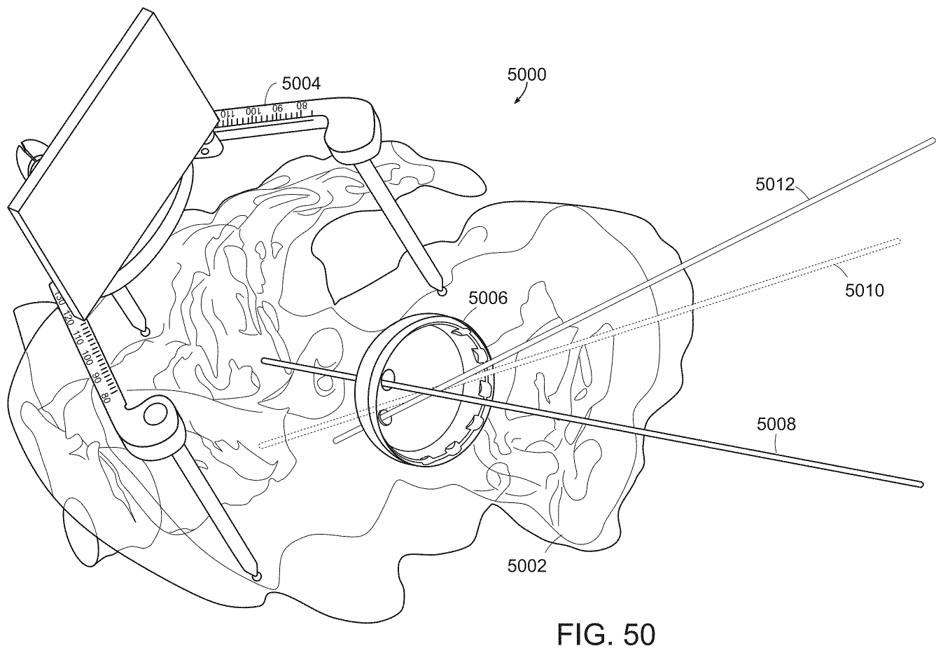

FIG. 50 is a pictorial representation of an example hologram 5000 in accordance with one or more embodiments. The hologram 5000 may be a composite hologram that includes a hologram 5002 of the patient's pelvis, a hologram 5004 of a registration and tracking device as docketed to the patient's pelvis in a planned manner, a hologram 5006 of a shell implanted in the acetabulum of the pelvis, as planned, and holograms 5008, 5010 and 5012 of screw trajectories for the screws installed at the screw holes of the shell, as planned. The hologram 5000 may be co-registered with the patient's physical pelvis. The surgeon may rotate the physical shell within the patient's acetabulum until physical shell is oriented as shown in the hologram 5004, thus achieving the planned placement of the physical shell in the acetabulum. For example, the surgeon may rotate the physical shell until the screw holes of the physical shell are aligned with the screw holes shown on the hologram of the shell. With the physical shell positioned as planned within the acetabulum, the surgeon may install the physical bone screws to fixedly hold the physical shell in place. The surgeon may use physical bone screws having lengths as determined during the planning stage to ensure that none of the physical bone screws will extend beyond the surface of the patient's pelvis.

To install the physical bone screws along the planned trajectories, thereby securing the physical shell to the patient's pelvis, the surgeon, while wearing the MR device, may move relative to the patient so that the trajectory for the current screw to be inserted is pointing at the surgeon.

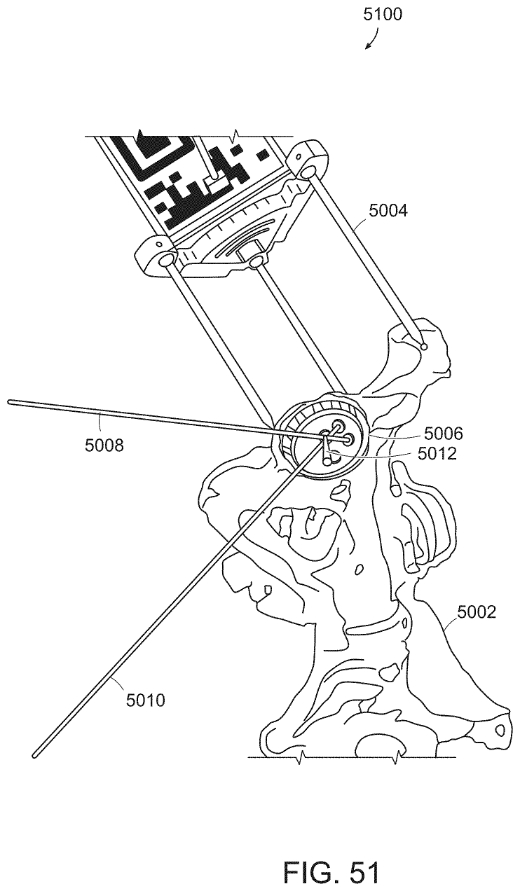

FIG. 51 is a pictorial representation of another example hologram 5100 in accordance with one or more embodiments. The hologram 5100 may include the hologram 5002 of the patient's pelvis, the hologram 5004 of a registration and tracking device as docketed to the patient's pelvis in a planned manner, the hologram 5006 of a shell implanted in the acetabulum of the pelvis, as planned, and the holograms 5008, 5010 and 5012 of screw trajectories for the screws installed at the screw holes of the shell, as planned. With the hologram 5100 of FIG. 51, the surgeon has moved such that the screw trajectory hologram 5012 is now pointing at the surgeon.

The surgeon may then align an axis of a drill or other screw installing surgical instrument with the trajectory hologram 5012 and install the physical screw keeping the axis of the drill or other surgical instrument aligned with the trajectory hologram. This process may be repeated with the other trajectory holograms 5008 and 5010, thus place all of the screws at the planned trajectories.

In some embodiments, one or more trackers may be attached to the drill, and the MR device may track the drill during the procedure. The MR device may present a hologram showing the drill trajectory if the drill were to be used in its current position. The MR device also may determine the available drill depth and/or screw length in the pelvis before piercing the surface of the bone, based on the current position of the drill, which may be different than the planned screw trajectory. The MR device may also present information regarding the difference between the planned screw trajectories, including the planned screw lengths, and the actual screw trajectories and available screw lengths within the bone, based on the position of the drill during surgery.

Automatic Determination of Screw Trajectories and Screw Lengths

As described, a surgeon may interact with the planning tool 1106, e.g., through one or more planning windows, to manually plan the placement of the physical shell, which in turn controls the screw trajectories, and also to select lengths for the bone screws to achieve one or more goals, such as using the longest screw lengths possible.

In some embodiments, the planning tool 1106 may automate part or all of the process of planning the screw trajectories, e.g., the placement the shell, and selecting screw lengths. For example, the planning tool 1106 may determine the maximum screw lengths for a range of rotational positions of the shell. This range may be a default range for all patients or the range may be entered by the surgeon. The planning tool 1106 may start its analysis with the shell model at a predetermined starting position, such as zero degrees. The planning tool may determine the maximum screw lengths that can be used at this starting position without having any of the screws extend beyond the pelvis and into the patient's tissue. The planning tool 1106 may then rotate the shell model a set amount, e.g., 1 degrees, and again determine the maximum screw lengths for this new rotational orientation of the shell. The planning tool 1106 may continue this process of repositioning the shell and determining the maximum screw lengths for the new shell orientation for the full rotational range of the shell. The planning tool 1106 may then present the solution that achieved the largest combined maximum of screw length. In some embodiments, the planning tool 1106 may present the top five, ten or some other number of solutions. The surgical planner may evaluate the solutions derived by the planning tool 1106 and select one of them for use in the surgical plan. The surgical planner may apply additional criteria when selecting one of the solutions presented by the planning tool 1106.

In some embodiments, the surgical planner may specify one or more constraints on the automated planning process performed by the planning tool 1106. For example, the surgical planner may indicate one or more preferred or desired screw lengths. The entered lengths may be the same for all screws or the surgeon may enter different lengths for the screws. In other embodiments, the surgical planner may specify minimum screw lengths for one or more of the screws used to secure the shell. The planning tool 1106 may then place the shell model at a default or initial position, e.g., orientation, within the acetabulum of the pelvis model for the patient. The planning tool may then determine the maximum screw lengths that can be achieved at the current orientation. If the screw lengths do not meet the constraints set by the surgical planner, the planning tool 1106 may discard the solution associated with the current orientation of the shell. The planning tool 1106 may then move the shell to the next orientation and determine whether the maximum screw lengths meet the constraints. If so, the solution may be saved by the planning tool 1106. If not, the planning tool 1106 may discard the solution. After evaluating each orientation in the range, the planning tool 1106 may present the solutions that met the constraints. The surgical planner may select one or more of the solutions and include the selected solution in the surgical plan.

It should be understood that additional criteria may be specified. For example, the surgical planner may direct the planning tool 1106 to find solutions for which one or more of the screws are located in one or more portions of the pelvis. For example, the surgical planner may select one or more portions of the pelvis that have high density and thus provide strong fixation of the bone screws. The planning tool 1106 may only save those solutions for which one or more screws are disposed in the one or more portions of the pelvis selected by the surgical planner. Alternatively, the surgical planner may select one or more portions of the pelvis that are to be avoided. In this case, the planning tool 1106 may only save those solutions for which one or more screws do not extend into the one or more portions to be avoided.

In some embodiments, the preoperative patient data may include information indicating bone density of the patient. For example, the patient data, such as a preoperative CT study, may include a Hounsfield unit for each voxel corresponding to the patient's pelvis. The Hounsfield units, which are dimensionless, may be used with the screw lengths to quantify the relative screw purchasing power, fixation, and/or pull-out strength of multiple screws. For example, a longer screw in lower density bone may provide the same screw purchasing power, fixation, or pull-out strength as a shorter screw in higher density bone. Information on screw purchasing power, fixation, or pull-out strength may be included in the surgical plan.

If the planning tool 1106 is unable to find any orientation of the shell where the screws meet the specified constraints, the planning tool 1106 may issue a message to the surgeon that no solution was found. The surgeon may alter one or more of the constraints, such as shorten one or more of the screw lengths and the process may be repeated. The surgeon may continue to shorten one or more of the screw lengths until the planning tool 1106 finds a solution.

It should be understood that the planning tool 1106 may apply other algorithms or procedures for automatically determining the screw trajectories. For example, in other embodiments, the surgeon may enter a position, e.g., orientation, of the shell model in the acetabulum of the pelvis model. The planning tool 1106 may then evaluate different screw lengths to determine the longest screw lengths associated with each screw hole of the shell model whereby the screw remains within the pelvis and does not extend beyond the pelvis. The planning tool 1106 may enter the maximized screw lengths in the surgical plan 1114.

In other embodiments, the planning tool 1106 may employ one or more optimization algorithms to select a cup or shell orientation that maximizes one or more of the screw lengths while ensuring that none of the screws extend beyond the pelvis. The optimization algorithm may be constrained by a range through which the shell can be rotated within the acetabulum. The optimization algorithm may also be constrained based on the surface geometry of the pelvis such that none of the screws are permitted to extend beyond the surface pelvis. The optimization algorithm, as constrained, may then execute to find at least one solution for the trajectories of the screws that maximizes one or more of the screw lengths.

In some embodiments, the planned position and orientation of the cup within the acetabulum, which may have been determined for reasons other than optimizing screw length, may be input constraints to one or more optimization algorithms. The one or more optimization algorithms may then solve for a rotation of the cup that optimizes, e.g., maximizes, one or more selected characteristics, such as the total length, e.g., in millimeters, of screw purchase in the bone for all of the screws combined, for example. The one or more optimization algorithms may calculate the intersection of the screw trajectories with the bone surface automatically and may optimize cup rotation to optimize screw length. The one or more optimization algorithms may also consider angled screws as described above.

With the physical cup component implanted as planned, the surgeon may insert a liner into the cup component.

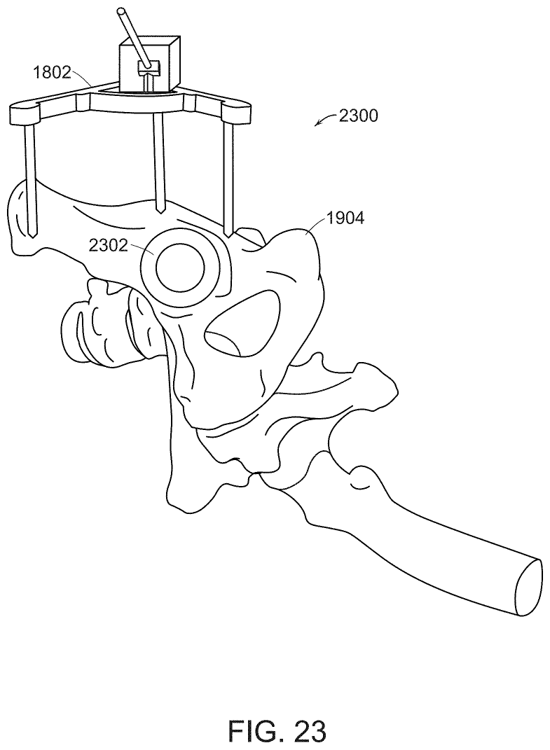

FIG. 23 is a pictorial representation of a hologram 2300 in accordance with one or more embodiments. The hologram 2300 may include the hologram 1802 of the HipXpert device, the hologram 1904 of the patient's pelvis, and a hologram 2302 of the cup component with liner implanted in the patient's acetabulum as planned. During the surgical procedure, the hologram 2300 may be co-located to the corresponding physical objects either manually and/or automatically, for example by co-locating the hologram 1802 of the HipXpert device with the physical HipXpert device. The surgeon may then examine the physical cup component with liner as implanted and see if the location and orientation of the physical cup component with liner matches the hologram 2302. If not, the surgeon may reposition the physical cup component and/or the liner until its location matches the hologram 2302.

Predicted Range of Motion and Impingement.

Preoperatively, the placement of the components and the trimming of specific osteophytes can be planned. In addition, range of motion of the hip joint with the planned components and the planned locations may be simulated and the composite range of motion (in all directions) until some type of impingement occurs may be calculated. This could be bone femur-bone pelvis, implant femur-bone pelvis, bone femur-implant pelvis, or implant femur-implant pelvis impingement.

During surgery, once the physical cup is implanted and the physical osteophytes removed, the MR device 200 may perform object recognition of the cup to determine the exact placement of the cup relative to the pelvis. The MR device 200 may determine where the physical cup and/or other implants are, and may further determine the shape of the bone after osteophyte trimming. The MR device 200 may then update the 3D surface model(s) of the pelvis and calculate a range of motion to impingement based on the location of the cup and/or other implants as implanted.

During the procedure, the surgeon may check that the physical HipXpert tool is still in alignment with the anchored hologram of the HipXpert tool. If the surgeon sees that the physical HipXpert tool is no longer co-located with the hologram of the HipXpert tool, the surgeon may reposition the hologram including the hologram of the HipXpert tool to co-locate the hologram with the physical HipXpert tool and/or may reposition the patient so that the physical HipXpert tool is co-located with the hologram that includes the hologram of the HipXpert tool. In some embodiments, the navigation system 1000 may keep the hologram co-located with the physical HipXpert tool automatically, for example using methodologies such as image or object recognition.

In some prior art surgical navigation systems, a surgeon needs to look away from the surgical site to a display in order to monitor the tracking of surgical tools. An advantage of the present disclosure is that the surgeon can keep his eyes trained on the surgical site while tracking one or more surgical tools.

In some embodiments, the surgeon may attach one or more tracking devices to the patient. For example, the surgeon may attach a weathervane type device or an object with one or more QR codes to the patient's pelvis. The surgeon may operate the MR device 200 to render a hologram of the one or more tracking devices, e.g., the weathervane, the HipXpert tool, and the pelvis. The surgeon may operate user interface elements provided by the MR device 200 to resize, move, and/or rotate the hologram of the weathervane/HipXpert tool/pelvis so that the hologram is co-located with the physical HipXpert tool attached to the patient's pelvis. The surgeon may operate the MR device 200 to peg or anchor the hologram of the weathervane/HipXpert tool/pelvis at that location within the operating room. The surgeon may adjust the physical weathervane until it is co-located with the hologram of the weathervane. Once the physical weathervane is co-located with the hologram of the weathervane, the surgeon may secure or fix the physical weathervane at that location. The surgeon may then remove the physical HipXpert device from the patient's pelvis. The surgeon may utilize the physical weathervane and/or the hologram of the weathervane to implant the prosthetic cup component at the planned orientation and location. For example, the weathervane (physical or hologram) may have an indicator that points along the planned orientation for the central axis of the prosthetic cup component. The surgeon may use the weathervane (physical or hologram) as a guide to implant the prosthetic cup component at the planned orientation and/or location.

In some embodiments, the weathervane or a QR cube may be randomly positioned space in the operating room. The systems and methods could regenerate new holograms on the fly that show representations of those objects by scanning where they are relative to other objects.

One or more of the holograms described herein may include the weathervane, which may be used as the registration tool in place of or in addition to the HipXpert tool.

With the cup component implanted at the planned location, e.g., depth and orientation, the surgeon may continue with the surgical procedure. For example, the surgeon may reduce the hip joint and close the incision. In other cases, the surgeon may remove the femoral head, implant a prosthetic stem, reduce the hip joint, and close the incision.

In some embodiments, the MR device 200 may utilize object detection to detect the cup component as implanted at the patient's acetabulum. In some embodiments, the cup component may include a notch or other physical feature from which its orientation may be determined by the MR device 200. The MR device 200 may register the pelvis based on the location of the cup component as detected. The MR device 200 may then utilize the cup component to anchor one or more holograms as planned relative to the pelvis. In some embodiments, once the MR device 200 detects the cup component, the HipXpert device may be removed. In other embodiments, registration of the pelvis may be transferred from the cup component to another object such as a tracker attached to the patient's pelvis. Thus, the MR device 200 may continue to anchor holograms as planned even if the cup component is no longer in view.

Automated Image Recognition: Example QR Code

In the described embodiments, a surgeon wearing the MR device 200 may manually register one or more of the holograms to corresponding objects in the operating room, such as the HipXpert tool.

In some embodiments, the object recognizer 1002 may be configured to detect and track an image, such as a barcode, which may be a two dimensional (2D) Quick Response (QR) code. For example, a QR code tracking tool is available in the Windows Mixed Reality driver for immersive VR HMDs, such as the HoloLens HMD with the VuForia Engine. The object recognizer 1002 may incorporate and/or utilize the Windows Mixed Reality driver for immersive (VR) HMDs

In some embodiments, one or more QR codes may be added to and/or incorporated into a registration and tracking tool, such as the HipXpert tool. The one or more QR codes may be arranged in a predetermined geometric relationship relative to the HipXpert tool. For example, a three-dimensional (3D) shape, such as a cube, may be mounted on the HipXpert tool and one or more QR codes may be placed and/or formed on the respective sides or faces of the cube. The object recognizer 1002 may detect at least one of these QR codes, such as the QR code on the side of the cube that faces the MR device 200. Other 3D shapes that may be used include pyramids, triangular prisms, cuboids, etc.

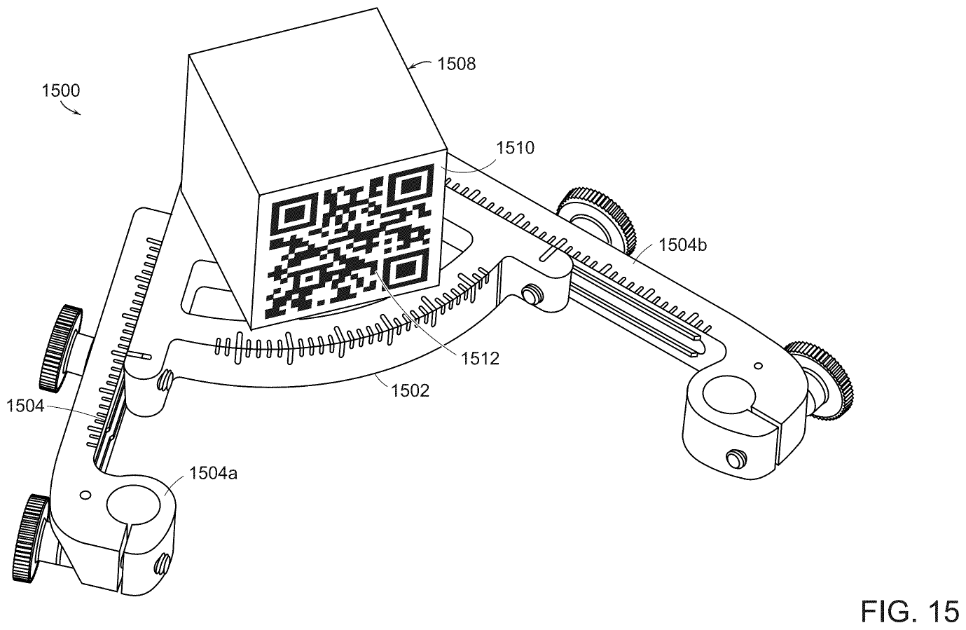

FIG. 15 is a pictorial representation of a portion of a registration and tracking tool 1500 in accordance with one or more embodiments. The tool 1500 may be a HipXpert tool with the compass and guide elements removed. The tool 1500 includes a hub 1502 and two arms 1504a and 1504b adjustably extending from the hub 1502. The tool 1500 further includes three (3) legs (not shown) that extend perpendicularly from a nominal plane defined by the hub 1502 and the two arms 1504a and 1504b. A first leg extends from the hub 1502 and second and third legs extend from ends of the two arms 1504a and 1504b. Mounted on the hub 1502 opposite the legs is a cube 1508. The cube 1508 may include a front surface 1510 carrying a QR code 1512. In some embodiments, QR codes may be placed on more than one side of the cube 1508, such as all but the side used to mount the cube 1508 to the hub 1502, e.g., the bottom side. In addition, the object recognizer 1002 may detect the QR code on the side of the cube 1508 that most closely faces the MR device 200. In some embodiments, the object recognizer 1002 may detect more than one QR code simultaneously to improve registration and/or tracking accuracy.

As described, the nominal plane of the defined by the hub 1502 and the two arms 1504a and 1504b may be parallel to the plane defined by the tips of the three legs. When docked to a pelvis, the tips of the three legs may define a patient-specific ipsilateral hemipelvic plane having a known geometric relationship to the AP Plane coordinate system for the pelvis. The nominal plane defined by the hub 1502 and the two arms 1504a and 1504b thus also has a known geometric relationship to the AP Plane coordinate system and/or to any other patient-specific coordinate systems chosen to be defined. Similarly, the cube 1508 is positioned on the tool 1500 to provide a known geometric relationship between the front surface 1510 of the cube 1508 which carries the QR code 1512.

A 3D model of the tool 1500 including the cube 1508 and the QR code 1512 may be generated.

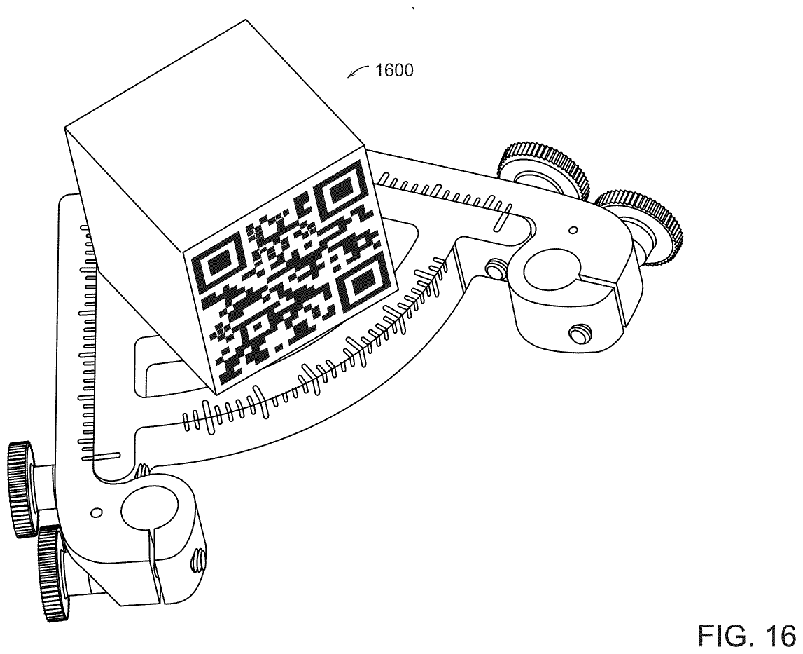

FIG. 16 is a perspective view of a portion of a 3D model 1600 of a registration and tracking tool in accordance with one or more embodiments. The 3D model 1600 corresponds to the physical registration and tracking tool 1500 including the cube 1508 having the QR code 1512.

In some embodiments, the model of the registration and tracking tool used in the pre-operative planning stage may correspond to the 3D model 1600. Similarly, the physical registration and tracking tool used during the surgical procedure may correspond to the physical registration and tracking tool 1500. The file(s) of the 3D model 1600 of the tool may be exported to a form from which the MR device 200 may generate one or more holograms.

During the surgical procedure, the object recognizer 1002 may search image or other data captured by the MR device 200 for the QR code(s) on the physical registration and tracking tool 1500. Upon detecting a QR code, the object recognizer 1002 may automatically co-locate, e.g., spatially align, the hologram of the registration and tracking tool with the physical registration and tracking tool with the QR code. Once the hologram has been co-located with the physical registration and tracking tool 1500, the surgeon may operate the MR device 200 to peg or anchor the hologram. In this way, the surgeon need not manually co-locate the holograms to the corresponding physical objects/devices. In some embodiments, when the application on the MR device 200 opens, the surgeon may identify, e.g., point to, a folder created for the patient that includes all planned holograms in the sequence of the procedure. When the MR device 200 identifies the QR code, a first hologram from the folder may be displayed in the right scale, position, and orientation. It should be understood that one or more of the holograms do not need to include the registration and tracking device itself, e.g., the HipXpert device. However, by including the HipXpert tool and the QR cube in the holograms, there is a constant visual confirmation to the surgeon that the anchoring is correct, e.g., because the physical HipXpert tool and the QR code, which sit outside of the patient's body, are co-located with the virtual images of those objects in the hologram.

In some embodiments, one or more applications (apps) may be created and loaded on the MR device 200. The app may include a planning application for running a surgical plan created for a patient and a navigation application for detecting a QR code and/or other object and presenting one or more virtual images, e.g., holograms. The app may be controlled through user interface elements provided by the MR device 200, such as hand gestures for opening and interfacing with applications. In other embodiments, a surgeon may control and/or operate the app using verbal commands. For example, in response to a first verbal command, e.g., “load”, the app may automatically open a file explorer window. The surgeon can then select a hologram file in a subfolder with a hand gesture. The app may automatically pick up a transformation matrix for the hologram, which may also be located in the same folder, identify the physical QR code in the surgical scene, and anchor the hologram. In other embodiments, the surgeon can use other verbal commands to cause the MR device to load and present additional holograms. Exemplary verbal commands include “hologram2”, “hologram3”, etc. for presenting the holograms in the planned order for the surgical procedure.

One or more components of the navigation system 1000 and/or the surgical planning system 1100 may be or may include software modules or libraries containing program instructions pertaining to the methods described herein, that may be stored on non-transitory computer readable media, and executed by one or more processors of a data processing device. In some embodiments, one or more components of the navigation system 1000 and/or the surgical planning system 1100 may each comprise registers and combinational logic configured and arranged to produce sequential logic circuits. In other embodiments, various combinations of software and hardware, including firmware, may be utilized to implement the present disclosure.

In some embodiments, one or more components of the navigation system 1000 and/or the surgical planning system 1100 may run on the MR device 200. During surgery, the surgeon may open the surgical plan using the surgical planning system 1100 running on the MR device 200. As described, the surgical plan may be updated based on the actual alteration of the acetabulum, the femur, or other bone or portion of anatomy and/or the actual placement of one or more implants.

In some embodiments, the MR device 200 may present one or more of the User Interfaces of the surgical plan in the operating room for review by the surgeon. For example, one or more of the User Interfaces may be presented on a wall or other surface of the operating room.

Transformation Matrices

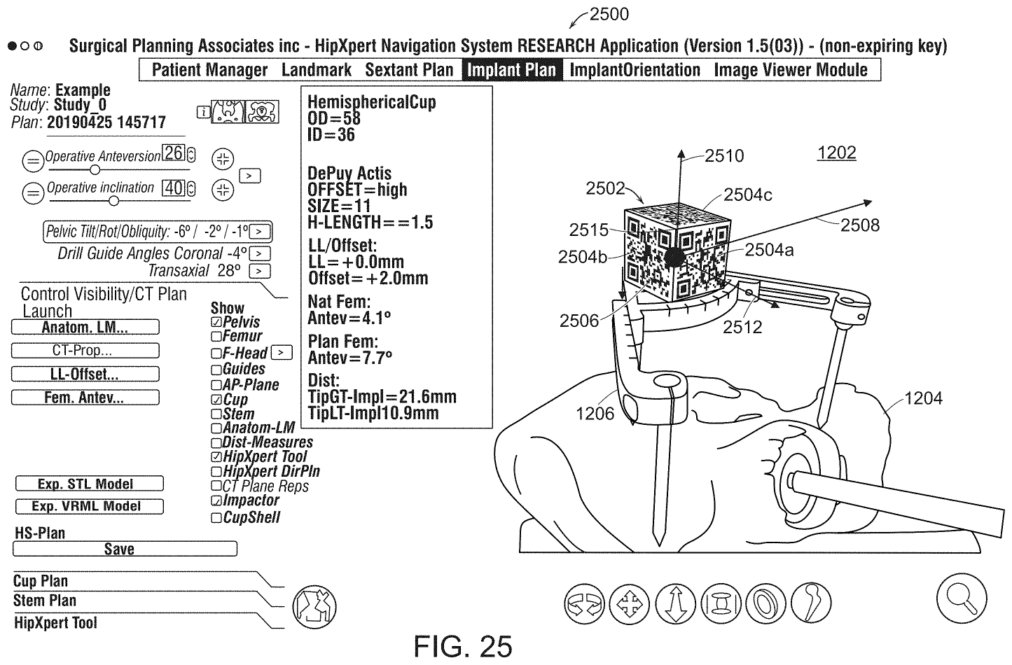

FIG. 25 is an illustration of an example planning window 2500 generated by the surgical planning system 1100 and presented on the display 1118 in accordance with one or more embodiments. The planning window 2500 includes a model pane 1202 presenting a 3D model of the patient's pelvis 1204. Docketed to the model of the pelvis 1204 is a 3D model of the HipXpert tool 1206. Mounted on the HipXpert tool 1206 is a cube 2502. The cube 2502 may include a plurality of faces, e.g., surfaces, carrying one or more QR codes, such as a front surface 2504a, a side surface 2504b, and a top surface 2504c. One or more coordinate systems may be established for the cube 2502. In some embodiments, a coordinate system may be established at the center of the cube 2502. For example, an origin, indicated at 2506 may be located at the center of the cube 2502 and x, y and z axes 2508, 2510 and 2512 may be defined relative to the origin 2506. The x, y and z axes 2508, 2510 and 2512 may be aligned with, e.g., by parallel to, respective edges of the cube 2502.

In addition, each QR code may expose a spatial coordinate system that is aligned with the QR code, for example at the top left corner of the finder pattern. As an example, the QR code 2504b may expose a spatial coordinate system indicated at 2515. It should be understood that the other QR codes may expose their own spatial coordinate systems. It should be understood that the spatial coordinate systems associated with the QR codes may be aligned at other locations besides the top left corner, such as the center of the QR codes, among other locations.

Because the cube 2502 is mounted on the HipXpert device 1206 and the HipXpert device 1206 is docked to the patient's pelvis, the cube 2502 is located in a fixed location in space relative to the patient's pelvis and thus relative to the AP Plane defined for the patient's pelvis (or any other chosen pelvic coordinate system). In some embodiments, the cube 2502 may always be mounted in the same way to the HipXpert device 1206 used for each patient.

In some embodiments, the planning tool 1106 generates one patient-specific transformation matrix that may be used in determining where to present the virtual images, e.g., holograms, created for a surgical procedure. For example, the planning tool 1106 may generate a patient-specific transformation matrix that determines the orientation and position of the virtual image, e.g., hologram, relative to the coordinate system established at the center of the cube 2502. In particular, the transformation matrix may specify the orientation and position of the hologram relative to the coordinate system that includes the origin 2506 and the x, y and z axes 2508, 2510 and 2512 defined for the front face 2504a of the cube 2502. This patient-specific transformation matrix may relate the coordinate system at the center of the cube to the random position of the patient in the CT scanner (or other image modality) from which the surface models of the patient's anatomy are generated.

In addition, a transformation matrix may be defined that relates the spatial coordinate system associated with each QR code to the coordinate system established at the center of the cube 2502. Because it is a cube, these transformation matrices may all be the same.

As described herein, during the surgical procedure, the MR device 200 may detect the QR code applied to one of the faces or surfaces of the physical cube mounted on the physical HipXpert device that is docked to the patient's pelvis. The MR device 200 may utilize the transformation matrix associated with the detected QR code and the patient-specific transformation matrix to orient and position the virtual image, e.g., the hologram. The MR device 200 may anchor the hologram relative to the coordinate system at the center of the cube. In some embodiments, the patient-specific transformation matrix may be stored in the folder with the holograms. The transformation matrix or matrices associated with the QR codes may be hard coded in the application or in other embodiments may also be stored in the folder. When the MR device 200 accesses a hologram from the folder for presentation, the MR device 200 may also retrieve the patient-specific transformation matrix.

As noted, a patient-specific transformation matrix may be defined for the holograms that will be presented during a surgical procedure. This patient-specific transformation matrix may be defined relative to a selected point of the cube 2502. The selected point may be the center of the cube 2502. As noted, the cube 2502 may be mounted to the HipXpert device, which in turn is docked to the patient's pelvis in a predetermined and known location. Accordingly, the center of the cube 2502 is in a fixed and known location relative to the patient's pelvis, e.g., relative to the AP Plane (or any other pelvic coordinate system). Locations and orientations of implants, e.g., the cup component, and tools, e.g., reamers and cup impactors, may be planned for a patient, e.g., relative to the AP Plane. Geometric relationships between these planned locations and orientations and the center of the cube 2502 may be determined. During surgery, the MR device 200 may recognize one or more of the QR codes on the physical cube of the HipXpert as docked to the patient. With the location of the physical cube in space determined, the MR device 200 can then use the patient-specific transformation matrix to determine where to locate the holograms such that the holograms appear in the planned locations and orientations.

In some embodiments, one or more secondary transformation matrices may also be defined. For example, secondary transformation matrices may be defined for each of the five QR codes applied to the faces of the cube 2502, e.g., front face, left face, right face, rear face, and top face. These secondary transformation matrices may provide geometric transforms from the respective QR code to the patient-specific primary matrix defined for the center of the cube 2502. When the MR device 200 detects a QR code (the particular QR code depending on the way the surgeon happens to be viewing the HipXpert device), the MR device 200 may retrieve the secondary transformation matrix associated with the detected QR code. The MR device 200 may then utilize this secondary transformation matrix together with the patient-specific transformation matrix to orient and position the respective hologram. While the transformation matrix generated for the center of the cube 2502 may be patient-specific, the secondary transformation matrices are not patient-specific. Instead, the secondary transformation matrices are the same for each cube geometry, e.g., dimensions. Thus, assuming the same cube 2502 is being reused or a cube 2502 with the same dimensions is being used with another patient, the same secondary transformation matrices may be re-used.

In sum, just a single patient-specific transformation matrix between the orientation and position of the QR code and the orientation and position of the rest of the hologram for every hologram that is to be presented may be generated. With the present disclosure, by detecting in space a QR code (that is on a cube mounted on a HipXpert device docked to a patient's pelvis), the MR device 200 can automatically register and track the patient's pelvis and allows for the presentation of one or more co-located holograms. In particular, the tips of the legs of the HipXpert device when docked to a patient's pelvis may define a hemi-pelvic ipsilateral reference plane having a known geometric relationship to the AP Plane. Furthermore, the frame of the HipXpert device from which the legs extend may be parallel to this hemi-pelvic ipsilateral reference plane (and thus have a known geometric relationship to the AP Plane). The cube which carries the one or more QR codes may be mounted on this frame. Accordingly, by detecting a QR code, the pelvis may be registered and tracked.

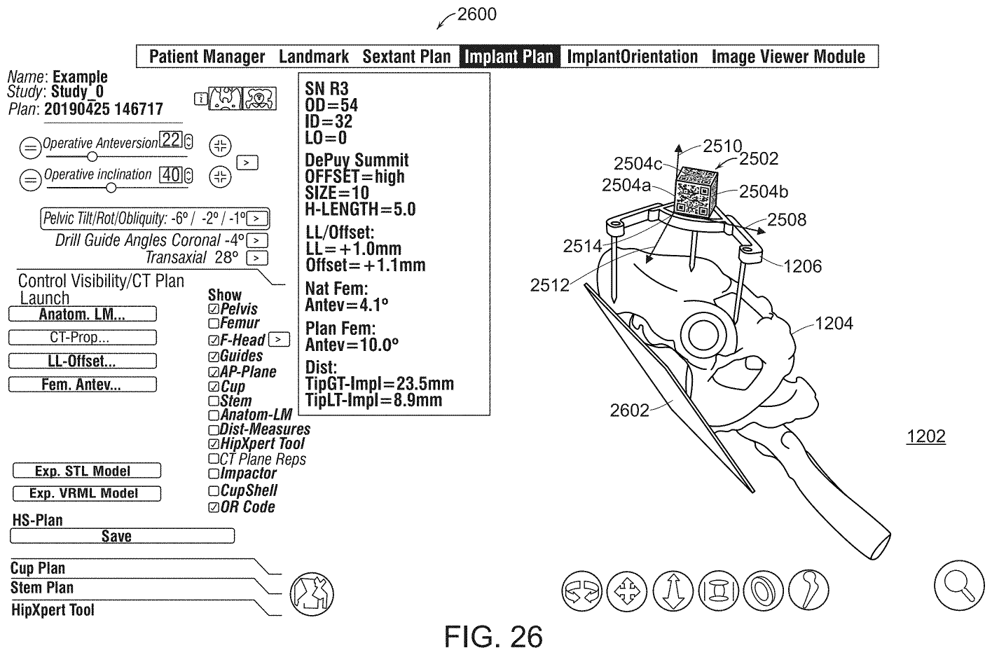

FIG. 26 is an illustration of an example planning window 2600 generated by the surgical planning system 1100 and presented on the display 1118 in accordance with one or more embodiments. The planning window 2500 includes a model pane 1202 presenting a 3D model of the patient's pelvis 1204. Docketed to the model of the pelvis 1204 is a 3D model of the HipXpert tool 1206. Mounted on the HipXpert tool 1206 is the cube 2502. An AP Plane 2602 is defined for the pelvis 1204.

As described, the QR cube may be mounted on a central portion of the frame of the HipXpert device. Because the legs of the HipXpert device may be of fixed lengths, the location of the QR cube and thus QR code(s) is constant from one patient to another. A patient-specific transformation matrix instructs the system as to where the QR cube and QR code(s) are located in space relative to random image-space coordinate system and also the anterior pelvis plane coordinate system. This transformation matrix is then a predetermined “patient-specific pass-code”. When the holograms are exported, the “key” or patient specific transformation matrix is also exported, which is used to determine where to present the holograms in space for that patient's specific surgical plan.

Cross-Section Display of Image Data Such as CT or MR Data.

As described, images of a patient such as a CT or MR study may be taken of a patient during a preoperative phase. For example, for hip surgery, a CT scan may be taken of the patient's pelvis and hips (with some images of the distal femur for coordinate system development). Such image modalities create an image volume that can be displayed as sequential slices in the original image acquisition plane, or can be displayed in any cut plane through the image volume. In fact, the display need not be a perfect plane, the image sampling could be made in any desired shape. For the purposes of this discussion the images could be generated as planar images. In addition, the image volume may be used to construct a 3D surface model, e.g., of the patient's pelvis. The 3D surface model may be opened and manipulated using a CAD software environment. Pre-operative planning may be performed using the 3D surface model. For example, the 3D surface model may be used to plan the preparation of bone surfaces and the selection, location and orientation of one or more prosthetic implants.

In some embodiments, the entire image data volume such as a CT image volume for a patient or a portion thereof may be loaded onto or otherwise made accessible to the MR device 200. During surgery, the MR device 200 may display desired sub-sections of the image volume to the surgeon. For example, the MR device 200 may register the portion of the patient's anatomy being operated on using one of the registration methods described, such as the patient's pelvis, and then tracked using a registration and tracking device such as a QR cube as described. The MR device 200 may then co-locate and anchor the entire image volume, such as a CT data volume, in space relative to the registration and tracking device. The system then may give the surgeon the option of seeing a portion of the image volume in space co-located with the actual location that the image data was acquired from on the patient. For example, the image volume could be cut in a planar cross-section that is perpendicular to the view of the surgeon wearing the MR device 200. That planar cross section could be determined as a fixed distance from the viewer or for example a fixed origin within the volume. For example, the surgeon, when preparing the acetabulum, may want to know the thickness of the remaining bone deep to the proposed cup placement. The origin of the cross section could be fixed at the center of the proposed placement of the acetabular component, and the displayed planar section through the volume would vary as the surgeon moves to stay perpendicular to the viewpoint of the surgeon's eyes.

For example, the CT data volume for the patient's pelvis may be co-located with the patient's pelvis in the operating room. The MR device 200 may generate one or more planar cuts through the CT data volume to produce a two dimensional (2D) CT image from the CT data. The MR device 200 may present this 2D CT image to the surgeon. The 2D CT image may be generated from a planar cut, also referred to as a cut plane, through a plurality of the slices included in the CT data volume. The planar cut through the CT data volume may be perpendicular to the surgeon's line of sight relative to the CT data volume as co-located with the patient's anatomy, e.g., the pelvis. By co-locating the CT data volume with the patient, the 2D CT image, as displayed by the MR device 200, may appear to the surgeon as overlaid on and co-located with the patient's anatomy. The cut plane may be set at a predetermined distance from the MR device 200. For example, if the surgeon moves his or her head and consequently the MR device 200 closer to the patient (e.g., lying supine on the operating table), the cut plane is moved backward (posterior) through the CT data volume. Similarly, as the surgeon moves his or her head away from the patient, the cut plane moves forward (anterior) through the CT data volume. Thus, by simply moving his or her head, the surgeon can control where the cut plane is formed in the CT data volume, and thus the resulting 2D CT image generated and presented by the MR device 200.

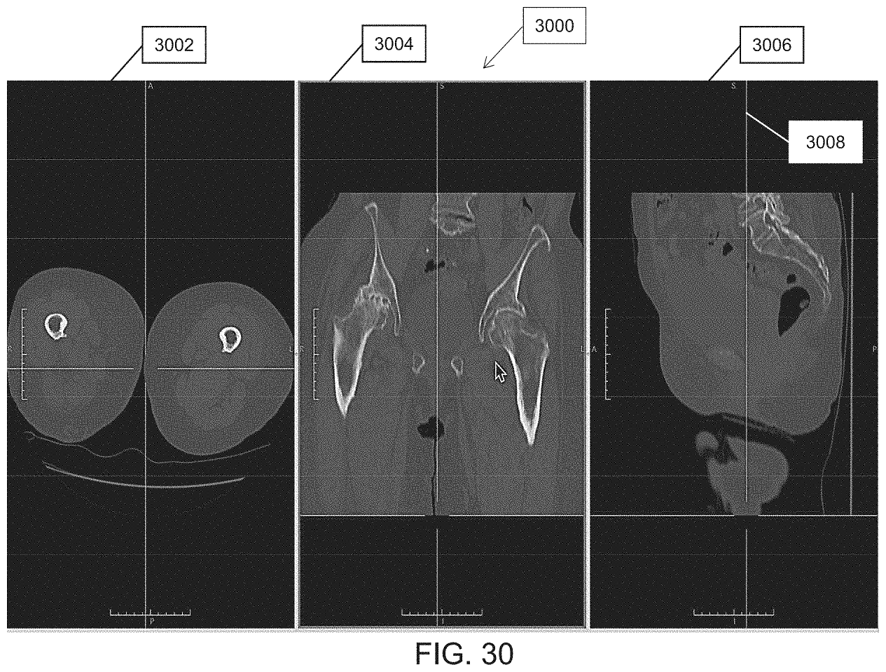

FIG. 30 is a pictorial representation of an example 2D CT image set 3000 of a patient's pelvis in accordance with one or more embodiments. The 2D CT image set 3000 may include an image 3002 through an axial plane, an image 3004 through a coronal plane, and an image 3006 through a sagittal plane. The coronal image 3004 shows the patient's left and right hip joints and a portion of the patient's spine. Suppose the patient is lying supine on an operating table, and the surgeon is looking down at the patient. The MR device 200 may generate and present a 2D CT image similar to the image 3004 through the coronal plane. The 2D CT image may be formed based on a cut plane indicated at 3008 on the sagittal image 3006 that is a predetermined distance from the MR device 200.

Now, suppose the surgeon moves his or her head away from the patient.

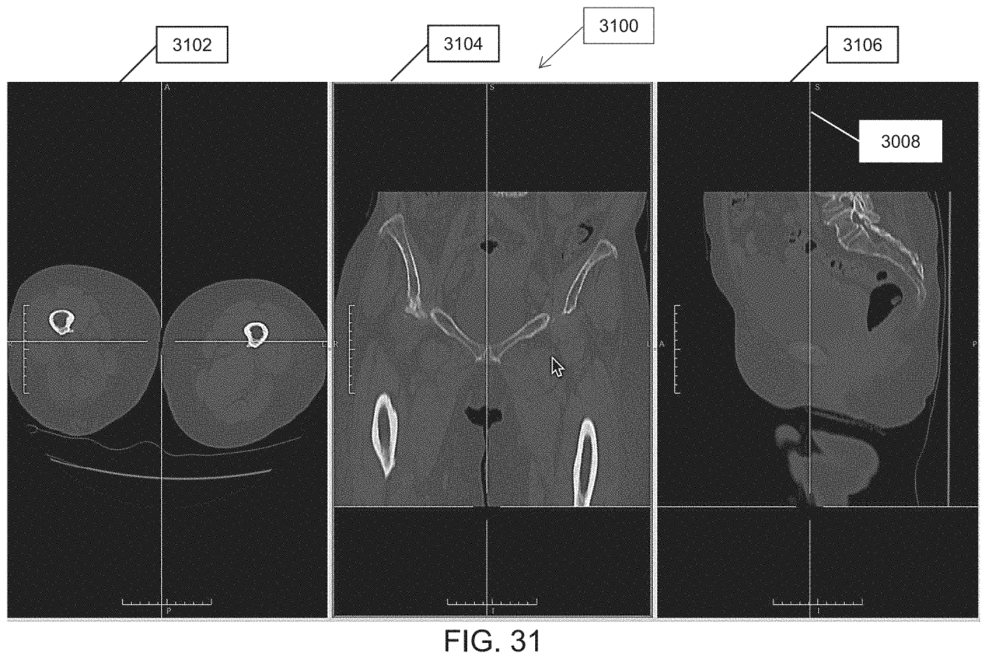

FIG. 31 is a pictorial representation of an example 2D CT image set 3100 of a patient's pelvis based on the new position of the surgeon's head in accordance with one or more embodiments. The 2D CT image set 3100 may include an axial image 3102, a coronal image 3104, and a sagittal image 3106. As illustrated, because the surgeon moved his or her head away from the patient, the cut plane 3108, which remains a fixed distance from the MR device 200, is moved anterior through the CT data. The coronal image 3104 is thus different than the coronal image 3004 (FIG. 30).

Now, suppose the surgeon moves his or her head closer to the patient relative to the distance producing the 2D CT image set 3000 (FIG. 30).

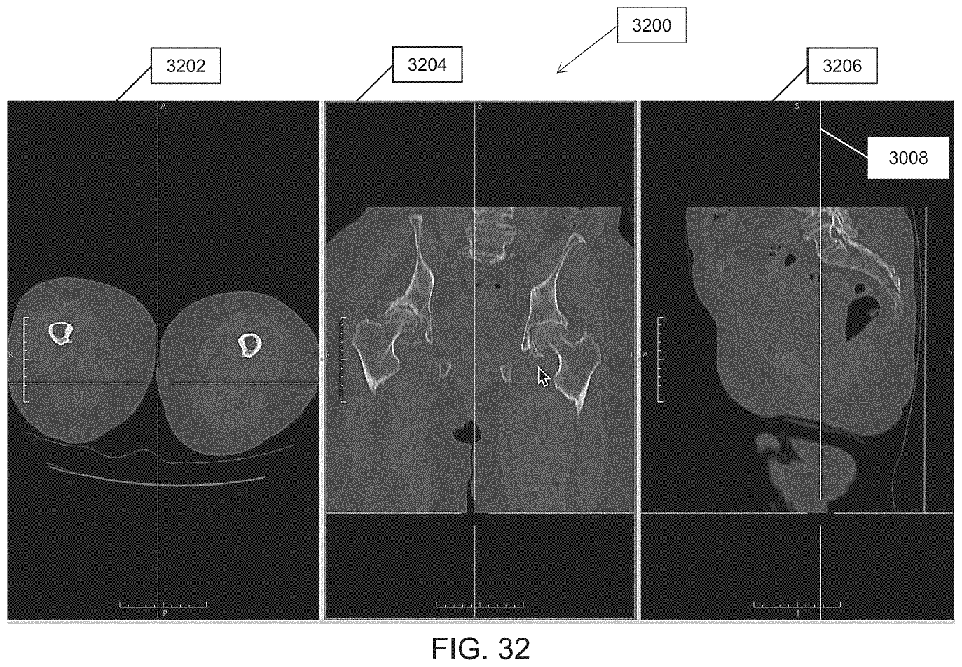

FIG. 32 is a pictorial representation of an example 2D CT image set 3200 of a patient's pelvis based on the new position of the surgeon's head in accordance with one or more embodiments. The 2D CT image set 3200 may include an axial image 3202, a coronal image 3204, and a sagittal image 3206. As illustrated, because the surgeon moved his or her head closer to the patient, the cut plane 3008, which remains a fixed distance from the MR device 200, is moved posterior through the CT data volume. The coronal image 3204 is thus different than the coronal images 3004 (FIG. 30) and 3104 (FIG. 31).

As noted, the 2D CT image generated and presented by the MR device 200 may be based on a cut plane that is a fixed distance from the MR device and perpendicular to the surgeon's line of sight. A suitable fixed distance is 50 cm for example. The 2D CT image is thus a cross-section of the CT data volume. In other embodiments, the 2D CT image data may correspond to one of the slices of the CT data volume.

In some embodiments, the MR device 200 may present one or more holograms in addition to the 2D CT image. For example, in addition to the 2D CT image, the MR device 200 may present one of the holograms including the reamer tool, the cup impactor tool, a cup component, etc. The presentation of one or more 2D CT images together with a hologram of a reamer may provide the surgeon with additional information, such as whether the reamer is getting close to reaming all the way through the inner wall of the patient's acetabulum. For example, while the MR device 200 presents a 2D CT image, the surgeon could intuitively determine how far the reamer has cut into the patient's acetabulum, e.g., by placing his or her finger in the wound while viewing the 2D CT image.

As the surgeon is reaming the acetabulum to prepare the cup bed for receiving the cup component, he or she may want to know how much bone is left behind the reamer medially, for example to avoid going through the bone. A cut plane that is along the surgeon's line of sight while reaming would provide this information. In some embodiments, the MR device 200 may present such a cut plane through the CT volume data. The cut plane display may be locked in position so that the surgeon may then move his or her head to observe the cut plane and thus see how much bone is left behind the reamer. In other embodiments, another medical professional in the operating room wearing an MR device 200 may observe this cut plane and inform the surgeon of how much bone is remaining.

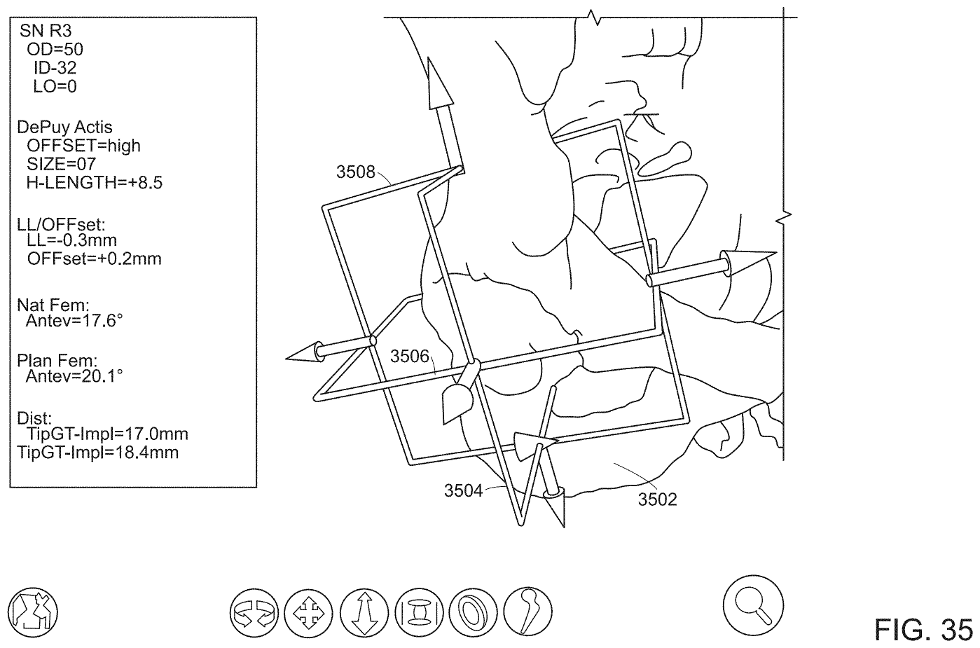

FIG. 35 shows a surface model of the pelvis 3502 with 3 cut planes. The green box 3504 signifies one image-generation plane, the red box 3506 signifies a second image-generation plane, and the yellow box 3508 signifies a third image-generation plane.

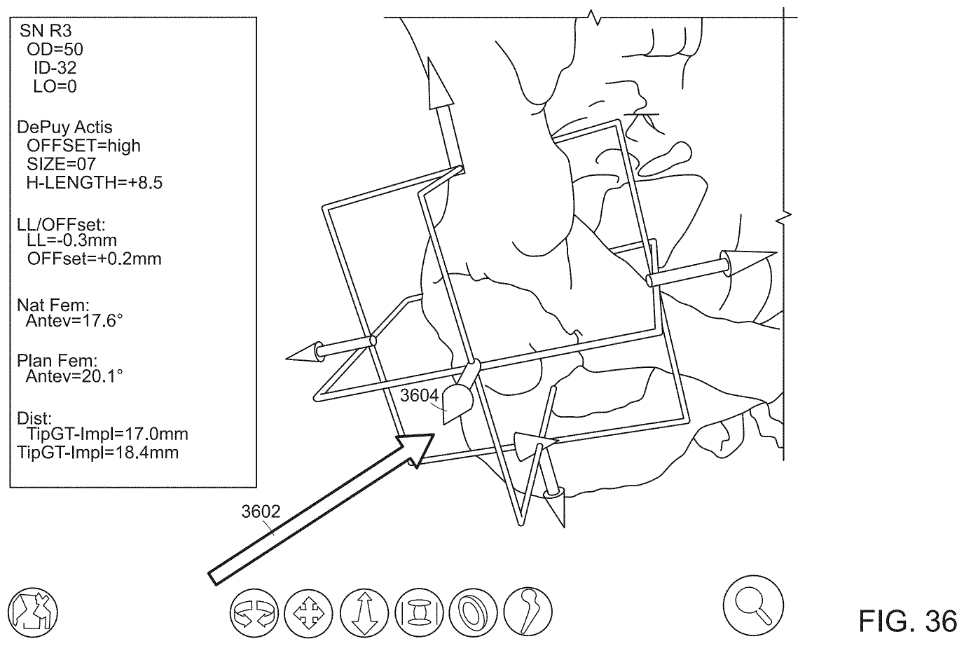

FIG. 36 shows a purple arrow 3602 pointing to a particular red arrow 3604 from the same image as illustrated in FIG. 35. A surgeon might often view the hip from the perspective of the designated red arrow 3604.

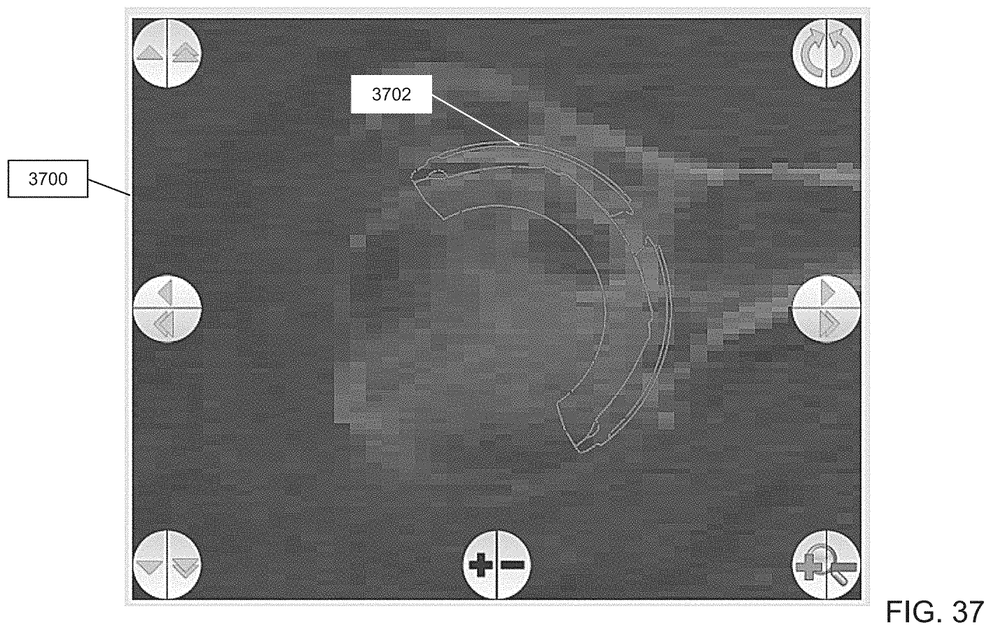

FIG. 37 is a pictorial representation of an image 3700 projected by the MR device 200 in the exact location within the patient's body that the data were acquired from. This image 3700 represents an image generated in the yellow box plane 3508 of FIG. 35 in that it is both perpendicular to the surgeon's viewpoint and in a plane that includes the center of the planned acetabular component. FIG. 37 also shows a cross section of the planned acetabular component indicated at 3702 that could be turned on or off depending upon the surgeon's preference.

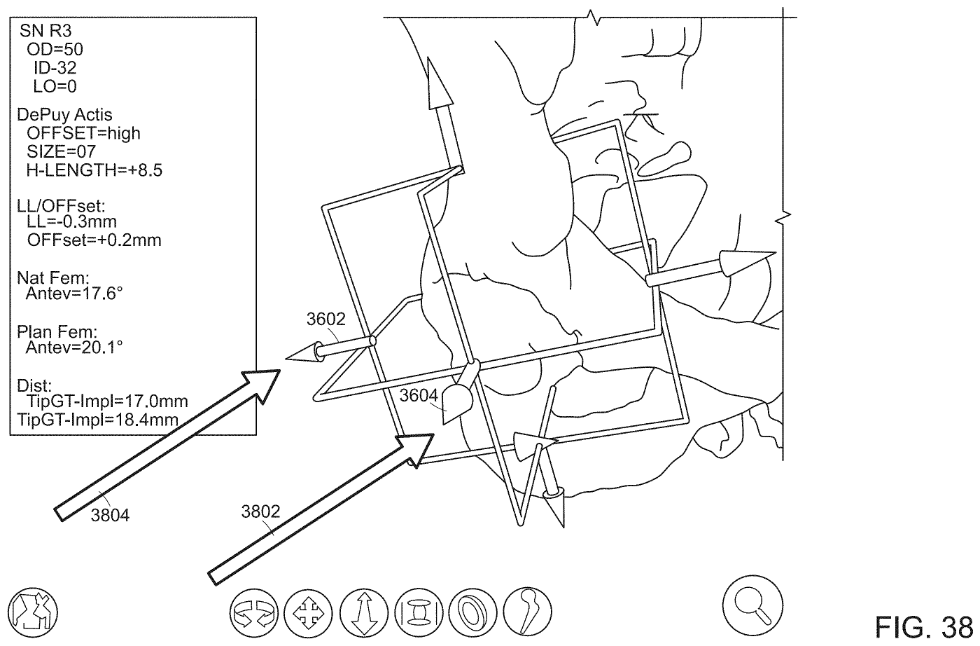

FIG. 38 shows the original surgeon's viewpoint (the red arrow 3604 designated by the purple arrow 3602) and a potential second viewpoint that is the red arrow 3802 designated by the light blue arrow 3804.

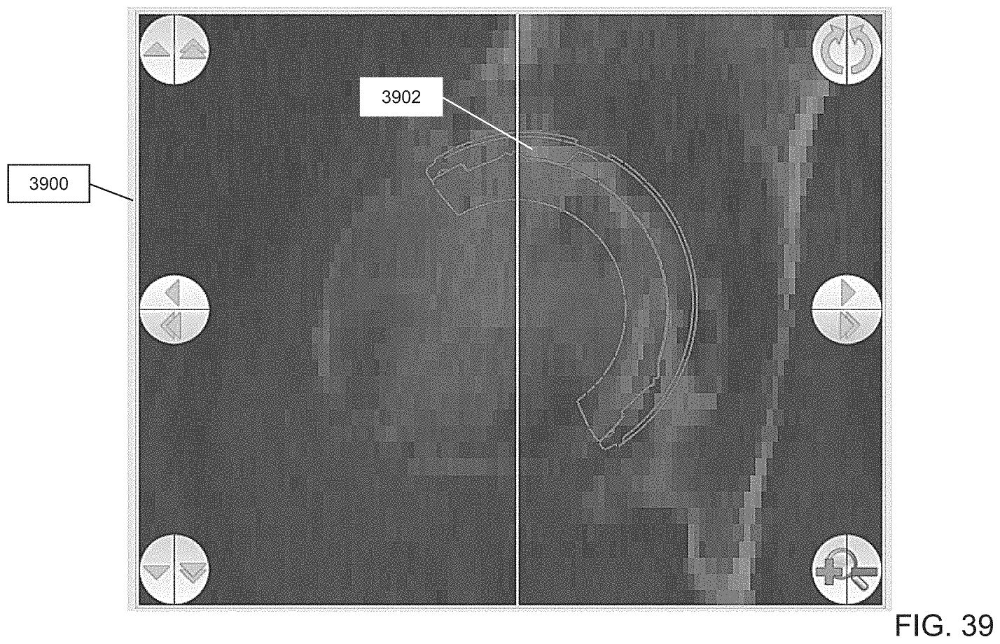

FIG. 39 is a pictorial representation of an image 3900 generated by the MR device 200 of a cut plane in the plane of the green box 3504, being perpendicular to the surgeon's line of sight when viewing from the point of view of the red arrow 3802 that is designated by the light blue arrow 3804. The MR device 200 may display the image 3900 in the exact location from which the image pixels were acquired from inside the patient's body at the time that the CT study (or any other image study with such a dataset) was acquired.

As described, the MR device 200 may automatically display images that are perpendicular to the surgeon's viewpoint in real time as the surgeon moves his or her head around. The MR device 200 also may “hold” the display of an image in the green box 3504, e.g., in response to user input, and the surgeon wearing the MR device 200 may be able to move the device 200 around without causing a new image to be recalculated.

The MR device 200 may thus create and present images that are co-located with the actual patient, from any desired angle, depth, and shape. In addition, the image need not even be a planar image.

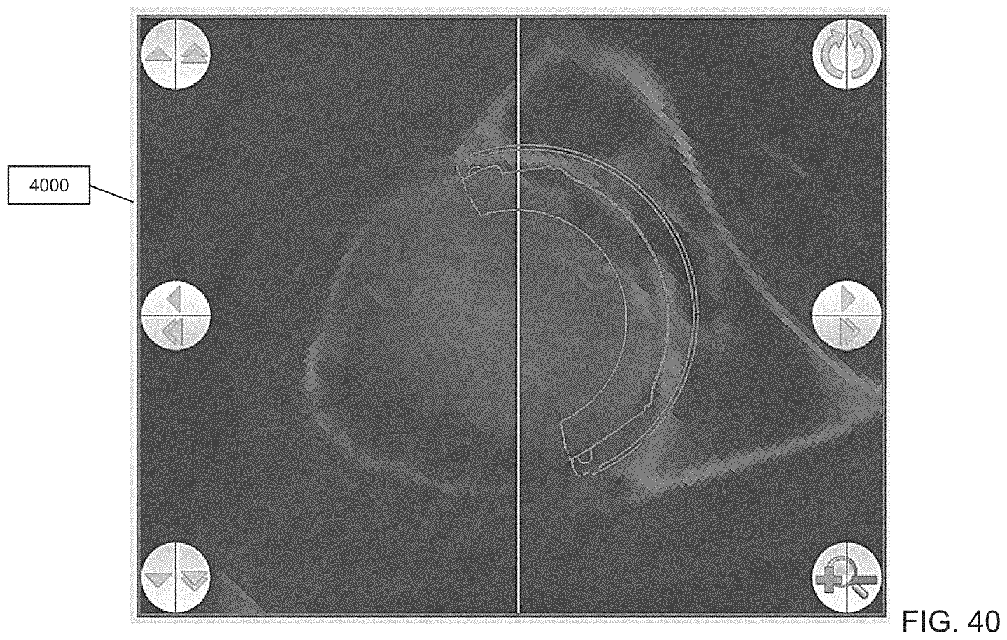

FIG. 40 is a pictorial representation of an image 4000 generated by the MR device 200 of a cut plane in the plane of the red box 3506.

In some embodiments, multiple planar cuts may be made through the CT volume data and presented by the MR device 200. For example, three orthogonal, planar cuts can be made in the CT volume data and presented by the MR device 200.

It also should be understood that the cuts made through the CT volume data need not be planar. For example, a curved cut or other shaped cut may be made through the CT volume data and presented by the MR device.

Multiple MR Devices

In some embodiments, more than one person in the operating room 100 may be wearing an MR device 200. For example, one or more assistants in addition to the surgeon 114 may be wearing MR devices 200. The MR device 200 worn by the surgeon may be the primary MR device, which may operate as a server, and the other MR devices may operate as clients of the primary MR device.

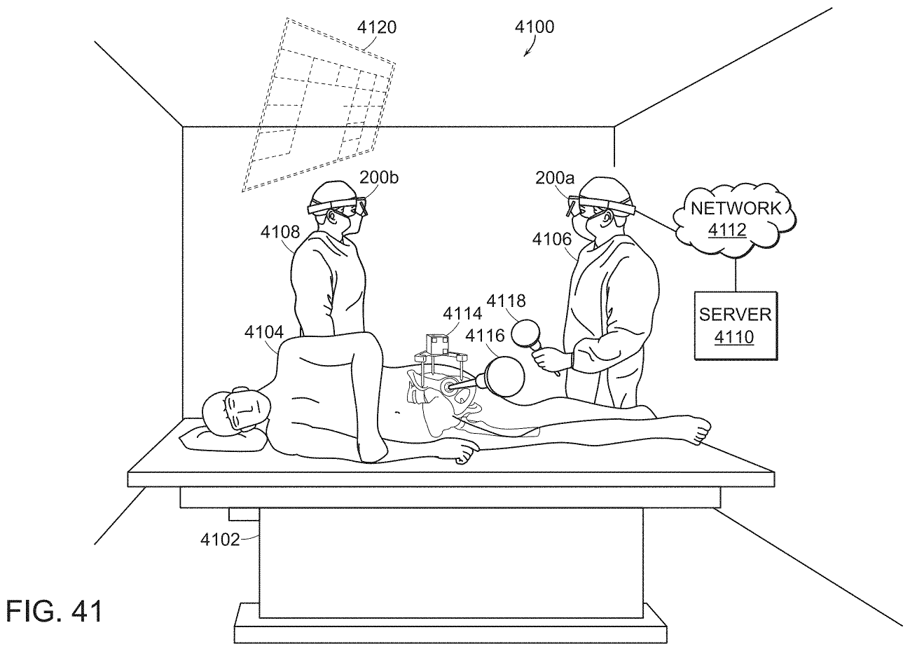

FIG. 41 is a schematic illustration of an operating room 4100 in accordance with one or more embodiments. Disposed in the operating room 4100 is an operating table 4102 on which a patient 4104 is positioned for a surgical procedure. A surgeon 4106 and at least one other medical professional 4108 may be in the operating room 4100. The surgeon 4106 and the medical professional 4108 may each be wearing an MR device 200a and 200b respectively. One or more of the MR devices, such as the MR device 200a, may be connected to a server 4110 via a network 4112. A physical registration and tracking device 4114 may be docked to the patient's pelvis. The MR devices 200a and 200b may present one or more virtual images, e.g., holograms, during the surgical procedure on the patient 4140. For example, a hologram 4116 of a cup impactor may be presented in a planned location relative the patient's pelvis. For example, the MR device 200a may detect the physical registration and tracking device 4114 and present the hologram 4116 of the cup impactor. The surgeon 4106 may guide a physical cup impactor 4118 to be aligned with the hologram 4116 to achieve one or more goals of the surgical procedure, such as implanting a prosthetic cup component at a planned location in the patient's pelvis. In some embodiments, one or more of the MR devices 200a and 200b may present a User Interface (UI), as indicated at 4120, in the operating room 4100, such as in space or against one or more walls of the operating room. The UI may be of a planning application presenting a surgical plan for the surgical procedure on the patient.

Automated Object Recognition and Registration of Tools and Body Structures.

The navigation system 1000 may receive data captured by one or more of the camera(s) on the MR device 200 of the surgical scene, such as image data in some embodiments. The object recognizer 1002 may detect an object in the received image data, and the object tracker 1006 may track the detected object. For example, the MR device 200 may transmit captured image data, e.g., via the network device 112, to the data processing device 100. The model database 1008 may be configured with data regarding the shape of the patient-specific HipXpert tool, such as three-dimensional (3D) shape for the HipXpert tool. As noted, the data may be one or more CAD files, 3D model data, etc. The object recognizer 1002 may search for an object in the received image data that matches this data, thereby identifying the HipXpert tool for example in the image data. The information in the model database 1008 may include the dimensions of the HipXpert tool on a patient specific basis, e.g., as adjusted for a specific patient, and may also know the location of the pelvis relative to the HipXpert tool, for example as determined during the surgical planning phase. The object recognizer 1002 may detect and/or recognize the HipXpert tool in a field of view, e.g., the image data, and the object pose detector 1004 may determine its orientation from which the navigation system 1000 may then calculate and track the location of the patient's pelvis in space. The object recognizer 1002 may implement the Vuforia Engine and Vuforia Model Targets technology from PTC Inc. of Boston, MA.

The surgeon may affix a second object, e.g., a tracker attached to the patient's pelvis, that can then be tracked, and a calculation of the second object's location relative to the HipXpert tool can be made by the navigation system 1000. The location of the pelvis can then be determined relative to this second object, allowing the HipXpert tool to be removed. That is, the navigation system 1000 may recognize the HipXpert tool itself optically because its size and shape are known to the system 1000, and so “seeing” it from any angle would allow for the determination of exactly where the HipXpert tool is positioned and oriented in space. The dimensions of the HipXpert tool and the predicted docking of the HipXpert tool onto the patient's pelvis is patient-specific, so the system 1000 may need to be configured with those parameters on a patient-specific basis.

Other tools also can be tracked in space either by teaching the system the unique CAD geometry of the other tools or affixing an object that is more easily tracked to the tool to be tracked. This may be useful for a cup impactor or acetabular reamer. The same may be true for the femur or any instrument used on the femur. The femur may be registered by recognizing a unique small visible section of the surface with a tracker attached to it, as described. The navigation system 1060 may track the femur based on object recognition and tracking of the object. In some embodiments, a tracker may then be attached to the femur and tracking continued based on this tracker allowing the surgeon to change the femur surgically making it no longer recognizable while still allowing the femur to be tracked. The process may be called patient-specific shape recognition registration methodology.

As described, tracking may be performed using the spatial detection system provided by the MR device 200, such as the depth camera 230 and the IR emitters. For example, the navigation system 1000 may implement simultaneous localization and mapping (SLAM) utilizing the data generated by the depth camera 230. In other embodiments, tracking may be performed by two cameras of known relative orientation to allow for stereoscopic calculation. Further, the stereoscopic cameras could be affixed to the MR device 200 as described, while in other embodiments image data from the 3D detection system 108 may be used by the navigation system 1000 either alone or in combination with image data from the MR device 200. The advantage of acquiring the image information from the one or more cameras on the MR device 200 is that the surgeon always needs a primary line of site, giving the camera(s) of the MR device 200 the same line of site as the surgeon. This is in contrast to the situation with traditional infrared stereoscopic cameras where line-of-site competition between the surgeon and the camera can occur. The other advantage of having the camera(s) on the surgeon's head is that the viewpoint of the camera(s) relative to the surgeon's eyes is known so that an augmented reality display of virtual objects can be displayed in the same perspective that the real objects would be seen in (except that they would otherwise be invisible, being buried deep inside the body) except perhaps for small exposed subsections during surgery.

In other embodiments, other tools besides by the HipXpert tool may be used and recognized and tracked by the navigation system 1000.

To aid in detecting a patient-specific object and determining its orientation and/or pose, the object may be asymmetrical and/or uniquely recognizable within the surgical scene. For example, to the extent the object is a tool, the tool may be asymmetrical. To the extent the object is a body part, the body part may be asymmetrical. Nonetheless, symmetrical objects, such as body parts, and/or tools may be used in the present disclosure.

In some embodiments, the compass portion of the HipXpert device may be omitted or removed.

In some embodiments, a second object may be attached to the object, e.g., body part, or to the tool to aid in detecting the object or tool in the image data and/or in determining its orientation and pose. The second object may be attached to the object or the tool in known geometric relationships such that locating the second object and determining its orientation and/or pose can be used to determine the location and/or orientation of the object and/or tool, e.g., using one or more translations.

In further embodiments, one or more markings may be applied to the object and/or tool to aid in its detection and/or in determining its orientation and/or pose. For example, a checkerboard or other unique and/or recognizable pattern may be applied to the object.

During the planning stage, adjustments may be determined for the physical registration and tracking tool 1500 so that it will fit, e.g., be docked to the patient's pelvis, as planned. The adjustments may include how far to slide out the extendable arms 1504a and 1504b so that the tips of the legs contact the patient's pelvis at planned locations. Thus, the dimensions of the tool 1500 may vary from one patient to another. Nonetheless, the dimensions of the hub 1502 of the tool 1500 is identical for all patients, e.g., it is a static component of the tool 1500. Furthermore, as described, the cube 1508 may be attached to the hub 1502 of the tool 1500 in the same manner for all patients.

In some embodiments, the cube 1508 with the QR code(s) may be omitted from the tool 1500. With this embodiment, the MR device 200 may be configured to recognize the physical tool 1500 in the operating room. For example, the MR device 200 may recognize one or more portions of the physical tool 1500 that is the same for all patients, such as the hub 1502. In this way, the same recognition process may be used for all patients even though the tool 1500 also includes portions adjusted on a patient-specific basis, e.g., the extent to which the arms 1504a and 1504b are extended. A patient-specific transformation matrix may be determined relative to the static portion of the tool being recognized, e.g., the hub 1502. Providing a portion of a registration and tracking tool that is static, e.g., the same, for all patients, and configuring the MR device 200 to recognize this portion of the tool may be more efficient, e.g., in terms of planning, processing and memory resources, than individually configuring the MR device 200 for each patient to recognize the tool as a whole as adjusted for each patient.

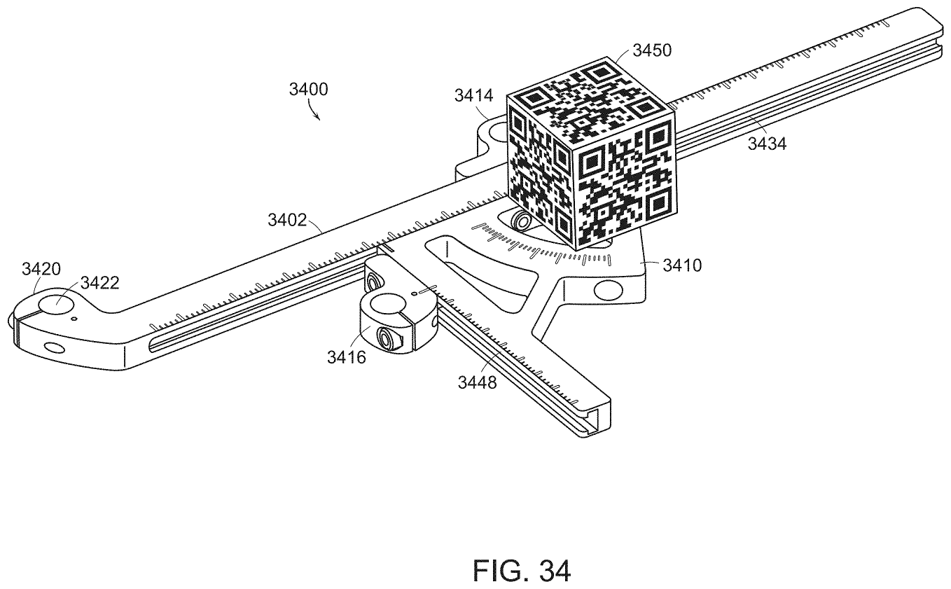

FIG. 34 is a perspective view of a hip registration and tracking tool 3400. The tool 3400 may include an elongated support arm 3402, a support frame 3410, a first moveable leg brace 3414, and a second moveable leg brace 3416. The elongated support arm 3402 may include a first end 3420. Disposed at the first end 3420 may be an opening 3422 configured to receive an end of a first leg (not shown) that may extend perpendicularly from the support arm 3402. An end of a second leg may be received at the first moveable leg brace 3414, and an end of a third leg may be received at the second moveable leg brace 3416. The second and third legs may also extend perpendicularly from the elongated support arm 3402, like the first leg.

A first track 3434 may be formed along at least a portion of a front side of the support arm 3402, and a second track (not shown) may be formed along at least a portion of a back side of the support arm 3402. The first and second tracks may be recessed tracks, such as slots or grooves. The support frame 3410 may include a first edge that engages the first track 3434 securing the support frame 3410 to the elongated support arm 3402, while allowing the support frame 3410 to slide along the front side of the elongated support arm 3402. The first moveable leg brace 3414, and thus the second leg, may be configured for slidable attachment to the back side of the elongated support arm 3402. The support frame 3410 may include a second edge 3448 to which the second moveable leg brace 3416 may slidably attach.

The first leg may have a tip configured to contact the right ASIS. Second and third legs may be slidably attached to the elongated support arm relative to the first leg. The distances between the first leg and the second and third legs may be determined preoperatively so that, when the second and third legs, are set to these predetermined distances along the elongated support arm, a tip of the second leg contacts the left ASIS, and a tip of the third leg contacts an anterior aspect of the ischium of the patient's pelvis below the acetabulum of the hip being operated on. An operating surgeon may access the patient's hip joint using the anterior approach or the anterolateral approach (e.g., with the patient in the supine position), and may dock the apparatus to the patient, thereby registering the patient's pelvis and establishing the patient-specific, supine pelvic reference plane and/or coordinate system.

Mounted to the support frame 3410 may be a cube 3450 with one or more QR codes. During surgery, the first moveable leg brace 3414 and the second moveable leg brace 3416 of the physical tool 3400 may be adjusted as planned so that the tips of the respective legs contact the patient's pelvis at the planned locations. The tool 3400 may be docked to the patient's pelvis. The MR device 200 may detect the one or more QR codes on the cube 3450 and may anchor one or more holograms as described herein.

The tool 3400 may be flipped over so that it may be used to operate on a patient's left or right hips. The support frame 3410 and the cube 3450 may also be flipped around so that it remains on top of the tool 3400.

Thus, the only things that may be specific for a patient when using a HipXpert registration and tracking tool or the tool 3400 are the arm lengths or the positions of the moveable leg braces, respectively, and the single patient-specific matrix, which may relate where the respective tool is in space to the raw image coordinate system from the CT scanner with the patient randomly placed within it.

In some embodiments, instead of utilizing a single tool that operates as a combination registration and tracking device, separate registration and tracking tools may be utilized. For example, a cube with one or more QR codes may be randomly attached to a patient's pelvis. A surgeon may then register the patient's pelvis, e.g., utilize a digitizing probe to digitize a plurality of points on the patient's pelvis. The location of the cube with the one or more QR codes may then be determined relative to the patient's pelvis as registered. The MR device 200 may then present one or more holograms in the planned locations and as anchored relative to the cube with the one or more QR codes.

It should be understood that other elements besides or in addition to a QR code may be used to register the pelvis or another anatomical structure, such as a tracker.

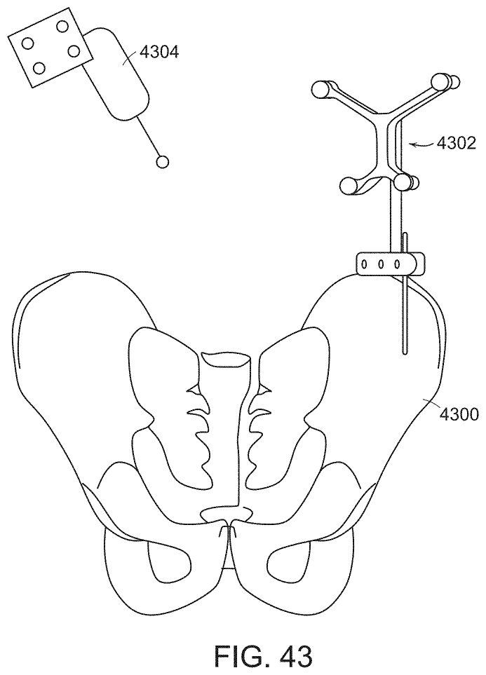

FIG. 43 is a schematic illustration of a front view of a pelvis 4300 in accordance with one or more embodiments. During the surgical procedure, a surgeon may attach a tracker 4302 to the pelvis 4300 at a random location. In some embodiments, the MR device 200 may recognize the tracker 4302 by virtue of its shape using object recognition and/or the MR device 200 may recognize an image on the tracker 4302, such as by way of example only a QR code. Alternatively, the tracker 4302 may include optical or magnetic elements that can be detected by the tracking system 106. The surgeon may utilize a digitizing probe 4304 to digitize a plurality of points on the surface of the pelvis 4300. The MR device 200 may similarly recognize the tracker using object and/or image recognition. Alternatively, the digitizing probe 4304 may include optical or magnetic elements that can be detected by the tracking system 106. The navigation system 1000 may process the digitized points to register the pelvis 4300. The navigation system 1000 may also track the pelvis 4300 via the tracker 4302 as detected by the MR device 200 or the tracking system 106. The MR device 200 may present one or more holograms anchored to the pelvis 4300 relative to the tracker 4302.

Augmented Reality for Hip Replacement Surgery:

Having the navigation system 1000 know where the pelvis is and having the navigation system 1000 know where the display is located in front of the surgeon's eyes allows for the detailed display of virtual images including computer models, e.g., of the pelvis and one or more tracked tools, from the same perspective as the surgeon. This would allow the surgeon to see the patient in reality, and also to see virtual objects such as the computer model of the pelvis projected onto the lenses of the MR device 200 in the same location as the actual object inside the patient.

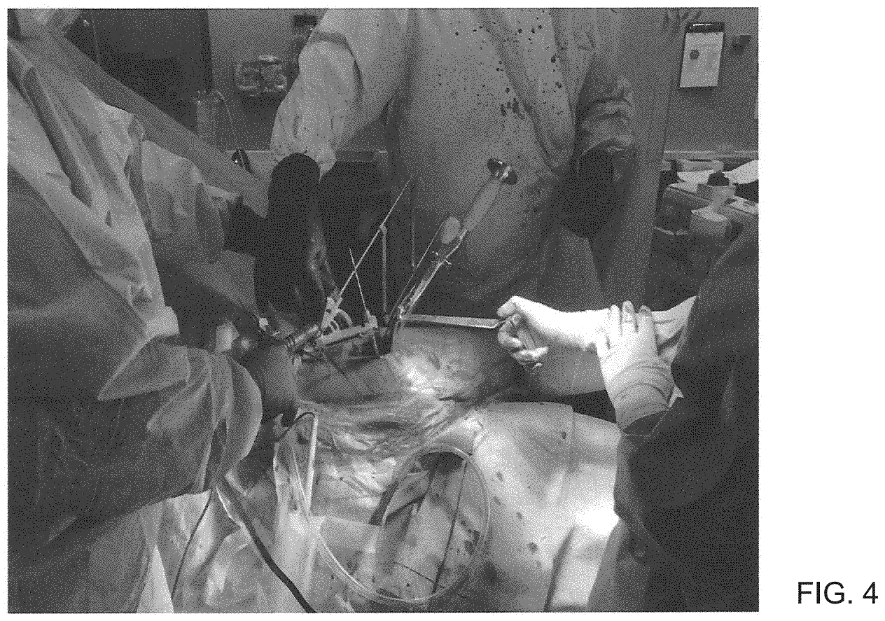

FIG. 4 is a pictorial representation of a surgical procedure showing a registration tool, e.g., the HipXpert tool, docked on a particular patient in accordance with one or more embodiments.

The location of the pelvis relative to the HipXpert tool may be known pre-operatively, e.g., during a planning phase. Using the spatial detection systems built into the MR device 200, the navigation system 1000 can calculate the perspective of the 3D object, e.g., the HipXpert tool, another tool, the patient's pelvis, another portion of the patient's anatomy, etc., from the surgeon's viewing perspective at that moment.

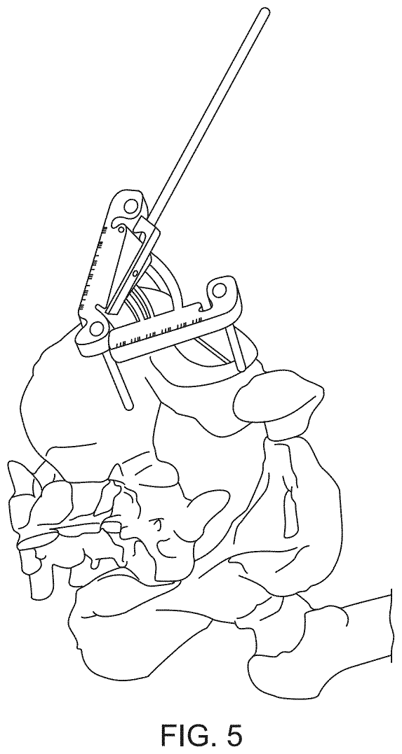

FIG. 5 is an illustration of a 3D surface model of a pelvis with a model of the registration and tracking device docked thereto in accordance with one or more embodiments.

Having calculated the surgeon's perspective of the tool and the pelvis, a virtual model of the pelvis can then be projected onto the lenses of the MR device 200 and thus within the surgeon's point of view in real time.

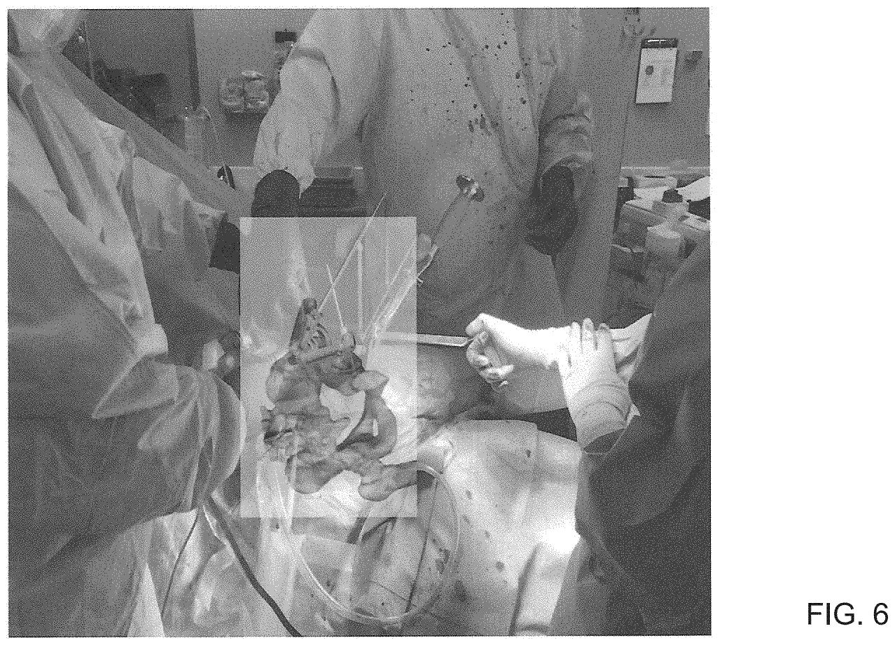

FIG. 6 is a schematic illustration of an image projected by the MR device 200 showing a virtual image of the patient's pelvis underneath the skin from the exact perspective of the surgeon at that moment in accordance with one or more embodiments.

Similarly, tools that are used on the patient could be seen in reality and a superimposed virtual model of the same tool in the same location could be projected by the MR device 200 for viewing by the surgeon. This would allow the surgeon to see the exact location of a part of the tool which, in reality, has disappeared inside of an incision, but yet a virtual image of which can be “seen” through the MR device 200.

Additionally, work that the tool accomplishes when being used can be tracked by the navigation system 1000 and the object that is changed can be updated. This would be true for example if a virtual display of the pelvis is projected as is a virtual display of an acetabular reamer. The camera(s) is able to track the relative locations of the two objects, and may also track and integrate an effect that the reamer has on the acetabulum, allowing for updating of the pelvis model to reflect the acetabular reaming itself and that could be compared both to the original structure and the planned structure of the acetabulum that the surgeon aims to achieve prior to implantation of the acetabular cup component. Accordingly, the navigation system 1000 may show the surgeon where s/he started, where s/he are so far, and where s/he needs to go next to accomplish to final goal of acetabular reaming.

Automated Object Recognition and Registration of Tools and Body Structures: Example: A Small Field of View Inside the Acetabulum

An alternative method of calculating the location of the pelvis in real time during total hip replacement surgery, for example, is to get a small view of the actual pelvis through the incision. Assuming the shape of the bone surface within that field of view is sufficiently unique, then the pelvis could be registered automatically by the navigation system 1000 just by “seeing” a small part of this patient-specific, unique object. For example, during total hip replacement, the femoral head is removed and the inside of the acetabulum is exposed. As long as the spatial detection system can see this bony structure, an automated shape registration of the entire bone could be accomplished.

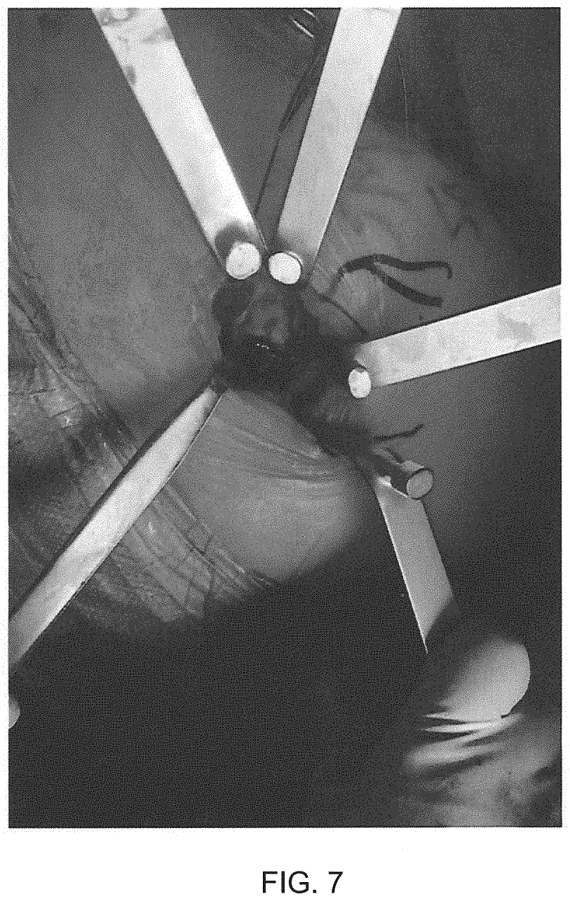

FIG. 7 is a pictorial representation of the view into the acetabulum of a patient through an incision during surgery in accordance with one or more embodiments.

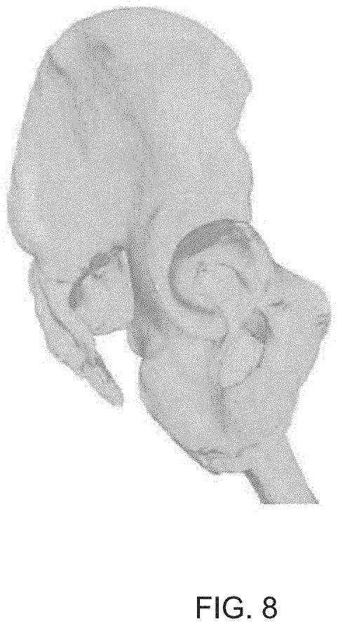

FIG. 8 is an illustration of a 3D surface model of the patient's pelvis from the same perspective as FIG. 7 in accordance with one or more embodiments. This matching registration can be done by the navigation system 1000, for example, by matching unique actual and virtual shapes together using object recognition.

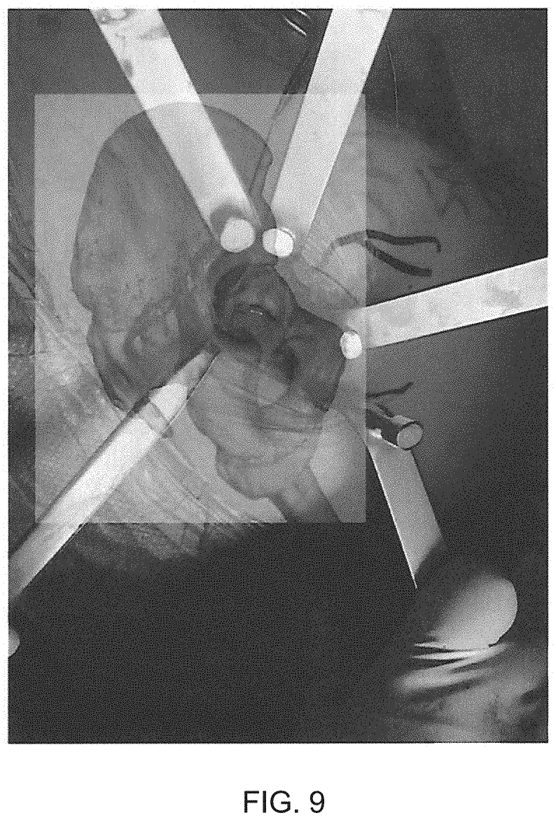

FIG. 9 is a schematic illustration of an image projected on the MR device 200 showing a virtual image of the patient's pelvis underneath the skin from the same perspective of the surgeon at that moment in accordance with one or more embodiments.

With existing systems, if instruments block the view or the bone surface is changed, then accurate registration and tracking is lost. In accordance with one or more embodiments of the present disclosure, this disadvantage can be avoided by attaching a separate tracker to the bone and transferring the relative information achieved through recognition of the patient-specific object and then simultaneous identification of the location of the separate tracker to the pelvis. Then, so long as the separate tracker can be tracked, surgery can proceed even though the surface that was used to achieve initial registration has been modified.

The system could combine the registration techniques depicted in FIGS. 4-6 and FIGS. 7-9 to achieve even greater accuracy.

Reality Feedback and Update Loop

In some embodiments, one or more anatomical structures may not be prepared in precisely the manner as planned. Nonetheless, a surgeon may determine that the partial preparation is acceptable, for example to achieve the one or more goals of the surgical procedure. For example, suppose a patient's acetabulum is prepared and a cup component implanted. However, suppose further that the cup component is not implanted exactly as planned, e.g., the position and/or orientation of the cup component within the acetabulum is somewhat different than the planned position and/or orientation. In some embodiments, the cameras or other sensors of the MR device 200 may be trained on the cup component as implanted. The object recognizer 1002 may detect and recognize the cup component. The navigation system 1000 may determine the position and/or orientation of the cup component as implanted and provide this information to the surgical planning system 1100. The surgical planning system 1100 may update the surgical plan for the patient using the actual position and/or orientation of the cup component as implanted, rather than the planned position and/or orientation. In other embodiments, the navigation system 1000 may determine the actual position and/or orientation of the cup component as implanted by determining a final location of the cup impactor. For example, the object recognizer 1002 may recognize the cup impactor while in its final location. The navigation system 1000 may determine the actual position and/or orientation of the cup component based on the final location of the cup impactor and the known geometry of the acetabular liner that is then inserted into the cup. For example, the navigation system 1000 may be configured with the geometric relationship between the cup impactor and the cup component. Thus, the navigation system 1000 can derive the position and/or orientation of the cup component from the position and/or orientation of the cup impactor. Alternatively or additionally, one or more trackers may be attached to the cup impactor, and the navigation system 1000 may determine the position and/or orientation of the cup impactor from the one or more trackers.

It should be understood that this is but one example of a reality feedback and update mode of the present disclosure. Feedback and updating the surgical plan may be performed with other elements besides the cup component.

In some embodiments, a sequence of holograms may be as follows:
1. pelvis and HipXpert device custom adjusted for the patient and docked to patient's pelvis, with the pelvis unchanged;
2. pelvis and HipXpert device custom adjusted for the patient and docked to patient's pelvis with the ideal cup bed as planned at the acetabulum;
3. HipXpert device custom adjusted for the patient (without pelvis), with ideal cup bed;
4-7. pelvis and HipXpert device custom adjusted for the patient and docked to the patient's pelvis and with a sequence of reamers and reamer handles in proposed locations. For example, if the planner wants to put in a 56 mm acetabular cup component, the planner might plan for the use of a 51 mm, a 53 mm, a 55 mm, and finally a 56 mm reamer. Each one of these reamers will do a certain amount of the work to achieve the final cup bed at the acetabulum. Holograms could be generated for each reamer and, during surgery, the holograms could be presented and the surgeon could work each reamer to match up with the hologram;
8. pelvis and HipXpert device custom adjusted for the patient and docked to the patient's pelvis, the cup component and the cup impactor with the screw holes of the cup component lined up in the planned orientation as the cup can be rotated around the handle. Alternatively or in addition, a hologram of the cup component and the cup impactor floating in space so that the surgeon can line up the screw holes perfectly rotationally;
9. pelvis and HipXpert device custom adjusted for the patient and docked to the patient's pelvis and the cup component and the cup impactor with the cup component located at the final location. Then, during surgery, with the physical cup impactor that matches the hologram, the surgeon would know that the cup component is in the planned, final location when the physical cup impactor and the physical cup component attached thereto line up perfectly with the hologram;
10. pelvis and HipXpert device custom adjusted for the patient and docked to the patient's pelvis and the cup component and the proposed screws for the cup component with planned directions and lengths to indicate to the surgeon the planned, e.g., optimal, direction to drill in and how long the screws will be;
11a and b. pelvis and HipXpert device custom adjusted for the patient and docked to the patient's pelvis and cup component showing with(a) and without(b) surrounding osteophytes to show the surgeon what to trim. Having planned removal of osteophytes, the systems and methods can determine what the potential impingement and/or free range of motion would be from the surgery and could show this information, for example based on degree of osteophyte removal; and
12. pelvis and HipXpert device custom adjusted for the patient and docked to the patient's pelvis and the cup component and the liner, e.g., the final product;

In some embodiments, the systems and methods may then do object recognition of the cup component and the pelvis to determine what the actual result of implantation is. Based on this information, the systems and methods could recalculate impingement and/or range of motion, i.e., on the spot, as desired.

Again, alternatively, the physical templates may be used as a registration and tracking device for subsequent navigation. An exemplary physical template is the acetabular template disclosed in U.S. Pat. No. 8,986,309 for an Acetabular Template Component and Method of Using Same During Hip Arthroplasty, which is hereby incorporated by reference in its entirety.

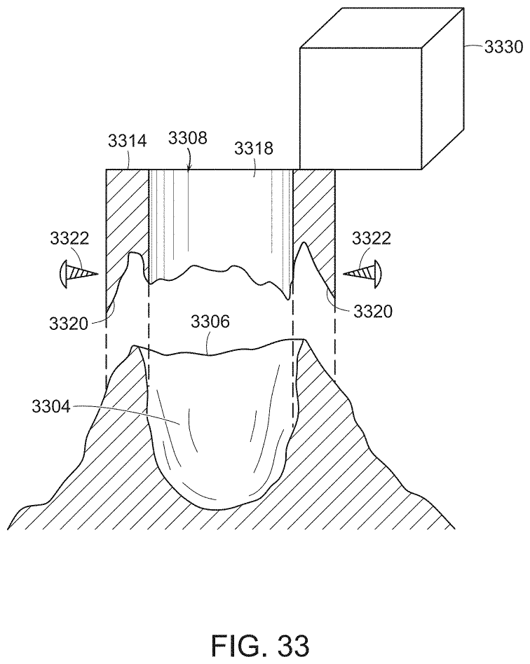

FIG. 33 is a partial side view of a patient's pelvis 3302 showing the patient's acetabulum 3304 and acetabulum rim 3306 with a custom fitted template 3308 in accordance with one or more embodiments. The custom fitted template 3308 may be generally circular shaped to mate with all or a substantial portion of the patient's acetabular rim 3306. Because the template 3308 matches the rough and uneven shape of the acetabular rim 3306, it fits to the rim 3306 and thus the pelvis in a single orientation. The template 3308 may have an upper surface 3314 and a lower surface 3320 opposite the upper surface 3314. Mounted on the upper surface 3314 may be a cube 3330 having QR codes (not shown) on at least some of its surfaces or faces. The lower surface 3320 is shaped to match the acetabular rim 3306. The template 3308 may have an open interior 3318 so that the template 3308 does not interfere with the placement of an acetabular cup component within the patient's acetabulum 3304.

The template 3308 may be held in place by one or more fasteners, such as screws 3322. With the template 3308 fitted to the patient's acetabulum, the MR device 200 may detect one or more of the QR codes on the cube 3330 and register the patient's pelvis. One or more patient-specific transformation matrices may be associated with the cube 3330 and/or QR codes and used to determine the orientation and position of virtual images, e.g., holograms, relative to a QR code and/or the cube 3330.

The present disclosure may use the spatial detection system of an augmented reality HMD for example to register and track anatomical structures and/or tools, for example by recognizing the three dimensional orientation of a portion of exposed anatomy, e.g., as viewed through an incision. As noted, for hip surgery, the HipXpert tool is tuned, e.g., adjusted, to the particular patient, and the navigation system 1000 is prepared to recognize that the HipXpert tool as adjusted for the patient within the image data of the surgical scene. Based on the detection of the patient specific object within the surgical scene, the navigation system 1000 may then register the rest of the “internal” scene, e.g., the patient's pelvis, another anatomical component or feature, etc.

Again, attaching a tracker to the bone would allow the registration information to be transferred to the tracker so that the surfaces that were originally used to achieve registration can be modified. This would allow for continued navigation and augmented reality display continuously from the surgeon's point of view no matter what that view is. In addition to trackers having optical or magnetic elements, such as the tracker 4302 illustrated in FIG. 43, a tracker may be a 2D or 3D shape that is spatially unique and thus recognizable by the MR device 200. Exemplary 3D shapes include an optical tracker without the reflective elements, e.g., just the arm elements. Exemplary 2D shapes include a metal plate having a non-symmetrical star shape or a non-symmetrical cross shape, etc.

As disclosed, in some embodiments, the present disclosure may replace the use of physical templates, such as templates used at the acetabulum. Instead, the system effectively presents a virtual template, such as a hologram of a template, that locks onto the patient's anatomy using patient-specific anatomical object recognition instead of an actual 3D printed physical template.

Patient-specific anatomical object recognition and CAD file automated surface matching registration methodology may replace use of a physical template. The CAD file of the patient specific anatomical object to be recognized may be prepared pre-operatively with the object then recognized in surgery by searching the data provided by the spatial detection system of the MR device 200 to determine and track the location of the object.

The object may also be tracked either directly or indirectly, e.g., through another object associated with the primary object, such as a tracker placed on the pelvis or the femur, among other options. Again, the tracking may be performed by the spatial detection system (e.g., cameras and/or other sensors) on the MR device 200, the tracking system 106, or the 3D detection system 108, among others. The present disclosure may also eliminate having to make and sterilize a physical template and instead could be planned immediately. The present disclosure may eliminate extensive digitization of surfaces that might otherwise be necessary for image-free or image-based navigation.

Combinations of registration techniques (such as digitizing the ankle landmarks or triangulating the center of rotation of the hip joint) could be employed to improve accuracy further.

It should be understood that additional and/or alternative registration techniques may be used. To improve the registration accuracy, the “virtual template” registration method may be combined with other methods. For the femur, triangulation of the center of rotation of the hip can be calculated by moving the hip around with a stereoscopic camera tracking the tracker attached to the femur. Combining this with the virtual template registration could further refine the accuracy of registration. Combining digitization with the virtual template registration could further refine the accuracy of registration.

The foregoing description of embodiments is intended to provide illustration and description, but is not intended to be exhaustive or to limit the disclosure to the precise form disclosed. Modifications and variations are possible in light of the above teachings or may be acquired from a practice of the disclosure. For example, while a series of acts has been described above with respect to the flow diagrams, the order of the acts may be modified in other implementations. In addition, the acts, operations, and steps may be performed by additional or other modules or entities, which may be combined or separated to form other modules or entities. Further, non-dependent acts may be performed in parallel.

Further, certain embodiments of the disclosure may be implemented as logic that performs one or more functions. This logic may be hardware-based, software-based, or a combination of hardware-based and software-based. Some or all of the logic may be stored in one or more tangible non-transitory computer-readable storage media and may include computer-executable instructions that may be executed by a computer or data processing system. The computer-executable instructions may include instructions that implement one or more embodiments of the disclosure. The tangible non-transitory computer-readable storage media may be volatile or non-volatile and may include, for example, flash memories, dynamic memories, removable disks, and non-removable disks.

The following examples implement one or more aspects of methods and/or systems of the present disclosure. These examples are non-limiting examples. Features of different examples may be combined in other implementations. Features of each example may be modified or removed in other implementations.
Aspect 1. A system comprising: a tracking device configured for attachment to a portion of a patient's pelvis; a computer-based surgical planning system configured to: present a two-dimensional (2D) or a three-dimensional (3D) model of the portion of the patient's pelvis; establish a coordinate system for the patient's pelvis; determine a location of one or more surgical tools relative to the coordinate system for the patient's pelvis; generate one or more files from which a plurality of holograms may be produced of: the 2D or 3D model of the portion of the patient's pelvis; and the one or more surgical tools; and a mixed reality (MR) head-mounted device (HMD), the MR-HMD including: at least one sensor configured to recognize the tracking device; one or more projectors configured to present the plurality of holograms; and a navigation system that tracks the tracking device and anchors the plurality of holograms in a space based on the coordinate system for the patient's pelvis, wherein the plurality of holograms indicate a planned trajectory of one or more bone screws for securing an acetabular shell to an acetabulum.
Aspect 2. The system of aspect 1, wherein the plurality of holograms further present information on the one or more bone screws.
Aspect 3. The system of aspect 1 or 2, wherein the information includes the length of the one or more bone screws.
Aspect 4. The system of any of the preceding aspects wherein the plurality of holograms include a hologram of a drill in a position and orientation for drilling a hole for the one or more bone screws or a screwdriver in a position and orientation for implanting the one or more bone screws at the planned trajectory.
Aspect 5. The system of any of the preceding aspects, wherein the tracking device mounted to a registration and tracking device configured to dock to the portion of the patient's pelvis in a predetermined location.
Aspect 6. The system of any of the preceding aspects, wherein the tracking device is randomly attached to the portion of the patient's pelvis, and the navigation system of the MR-HMD registers the patient's pelvis based on digitizing at least three predetermined points on the patient's pelvis.
Aspect 7. A system comprising: a tracking device configured for attachment to a portion of a patient's pelvis; a computer-based surgical planning system configured to: present a two-dimensional (2D) or a three-dimensional (3D) model of the portion of the patient's pelvis; establish a coordinate system for the patient's pelvis; determine a location of one or more surgical tools relative to the coordinate system for the patient's pelvis; generate one or more files from which a plurality of holograms may be produced of: the 2D or 3D model of the portion of the patient's pelvis; and the one or more surgical tools; a mixed reality (MR) head-mounted device (HMD), the MR-HMD including one or more projectors configured to present the plurality of holograms; and a navigation system that recognizes and tracks the tracking device and directs the MR-HMD to anchor the plurality of holograms in space relative to the coordinate system for the patient's pelvis, wherein the plurality of holograms indicate a planned trajectory of one or more bone screws for securing an implant to the patient's pelvis.
Aspect 8. The system of aspect 7, wherein the plurality of holograms further present information on the one or more bone screws.
Aspect 9. The system of aspect 7 or 8, wherein the information includes the length of the one or more bone screws.
Aspect 10. The system of any of aspects 7 to 9, wherein the plurality of holograms includes a hologram of a drill in a position and orientation for drilling a hole for the one or more bone screws or a screwdriver in a position and orientation for implanting the one or more bone screws at the planned trajectory.
Aspect 11. The system of any of aspects 7-10, wherein the tracking device is mounted to a registration and tracking device configured to dock to the portion of the patient's pelvis in a predetermined location.
Aspect 12. The system of any of aspects 7-11, wherein the tracking device is randomly attached to the portion of the patient's pelvis, and the navigation system registers the patient's pelvis based on digitizing at least three predetermined points on the patient's pelvis associated with a registration device configured for docking to the patient's pelvis.
Aspect 13. The system of any of aspects 7-12, wherein the navigation system is included in the MR-HMD.
Aspect 14. A computer-implemented method comprising: presenting a two-dimensional (2D) or a three-dimensional (3D) model of a portion of a patient's pelvis; determining a location of a registration device as docked to the 2D or 3D model of the portion of the patient's pelvis; establishing a coordinate system for the 2D or 3D model of the portion of the patient's pelvis based on the location of the registration device as docked to the 2D or 3D model of the portion of the patient's pelvis; determining trajectories of a plurality of bone screws for securing an implant to an acetabulum of the patient's pelvis, the trajectories determined relative to the coordinate system; generating one or more files for presenting holograms of the trajectories of the plurality of bone screws for securing the implant to the acetabulum of the patient's pelvis; and exporting the one or more files to a mixed reality (MR) head-mounted device (HMD) for presenting the holograms of the trajectories of the plurality of bone screws by the MR-HMD.
Aspect 15. The computer-implemented method of aspect 14, further comprising: establishing a coordinate system for the registration device; generating a transformation matrix between the coordinate system for the registration device and the coordinate system for the 2D or 3D model of the portion of the patient's pelvis; and exporting the transformation matrix to the MR-HMD.
Aspect 16. The computer-implemented method of aspect 14 or 15, further comprising: registering the patient's pelvis during a surgical procedure; utilizing the transformation matrix to anchor the holograms of the trajectories of the plurality of bone screws relative to the patient's pelvis.
Aspect 17. The computer-implemented method of any of aspects 14 to 16, wherein the registration device includes a tracking element and the registering the patient's pelvis includes: docking the registration device with the tracking element to the patient's pelvis; and recognizing the tracking element of the registration device.
Aspect 18. The computer-implemented method of any of aspects 14-17, further comprising: determining a length of at least one of the plurality of bone screws, wherein the length is determined to prevent the at least one of the plurality of bone screws from extending beyond the patient's pelvis; generating one or more additional files for presenting additional holograms of the length of the at least one of the plurality of bone screws; exporting the one or more additional files to the MR-HMD for presenting the additional holograms of the length of the at least one of the plurality of bone screws by the MR-HMD.
Aspect 19. The computer-implemented method of any of aspects 14-18, wherein the implant is an acetabular cup with two posterior screw holes and at least one additional screw hole and the determining the trajectories includes: planning a position and an orientation of the acetabular cup within the acetabulum; and with the acetabular cup at the planned position and orientation and with approximately 45 millimeter (mm) length bone screws placed at the two posterior screw holes and at the at least one additional screw hole, rotating the acetabular cup until bone screws at the two posterior screw holes are fully within the patient's pelvis; wherein the determining the length of the at least one of the plurality of bone screws includes reducing the length of the bone screw at the at least one additional screw hole until it is fully within the patient's pelvis.
Aspect 20. One or more computer-readable media comprising program instructions for execution by one or more processors, the program instructions instructing the one or more processors to perform operations according to the method of any one of aspects 14-19.

No element, act, or instruction used herein should be construed as critical or essential to the disclosure unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

The foregoing description has been directed to specific embodiments of the present disclosure. It will be apparent, however, that other variations and modifications may be made to the described embodiments, with the attainment of some or all of their advantages. Therefore, it is the object of the appended claims to cover all such variations and modifications as come within the true spirit and scope of the disclosure.

What is claimed is:

1. A system comprising:
a tracking device configured for attachment to a portion of a patient's pelvis;
a computer-based surgical planning system configured to:
present a two-dimensional (2D) or a three-dimensional (3D) model of the portion of the patient's pelvis;
establish a coordinate system for the patient's pelvis;
determine a location of one or more surgical tools relative to the coordinate system for the patient's pelvis;
generate one or more files from which a plurality of holograms may be produced of:
the 2D or 3D model of the portion of the patient's pelvis; and
the one or more surgical tools;
a mixed reality (MR) head-mounted device (HMD), the MR-HMD including
one or more projectors configured to present the plurality of holograms; and
a navigation system that recognizes and tracks the tracking device and directs the MR-HMD to anchor the plurality of holograms in space relative to the coordinate system for the patient's pelvis, wherein the plurality of holograms indicate a planned trajectory of one or more bone screws for securing an implant to the patient's pelvis.

2. The system of claim 1, wherein the plurality of holograms further present information on the one or more bone screws.

3. The system of claim 1, wherein the information includes the length of the one or more bone screws.

4. The system of claim 3, wherein the plurality of holograms includes a hologram of a drill in a position and orientation for drilling a hole for the one or more bone screws or a screwdriver in a position and orientation for implanting the one or more bone screws at the planned trajectory.

5. The system of claim 1, wherein the tracking device is mounted to a registration and tracking device configured to dock to the portion of the patient's pelvis in a predetermined location.

6. The system of claim 1, wherein the tracking device is randomly attached to the portion of the patient's pelvis, and the navigation system registers the patient's pelvis based on digitizing at least three predetermined points on the patient's pelvis associated with a registration device configured for docking to the patient's pelvis.

7. The system of claim 1, wherein the navigation system is included in the MR-HMD.

8. A computer-implemented method comprising:
presenting a two-dimensional (2D) or a three-dimensional (3D) model of a portion of a patient's pelvis;
determining a location of a registration device as docked to the 2D or 3D model of the portion of the patient's pelvis;
establishing a coordinate system for the 2D or 3D model of the portion of the patient's pelvis based on the location of the registration device as docked to the 2D or 3D model of the portion of the patient's pelvis;
determining trajectories of a plurality of bone screws for securing an implant to an acetabulum of the patient's pelvis, the trajectories determined relative to the coordinate system;
generating one or more files for presenting holograms of the trajectories of the plurality of bone screws for securing the implant to the acetabulum of the patient's pelvis; and
exporting the one or more files to a mixed reality (MR) head-mounted device (HMD) for presenting the holograms of the trajectories of the plurality of bone screws by the MR-HMD.

9. The computer-implemented method of claim 8, further comprising:
establishing a coordinate system for the registration device;
generating a transformation matrix between the coordinate system for the registration device and the coordinate system for the 2D or 3D model of the portion of the patient's pelvis; and
exporting the transformation matrix to the MR-HMD.

10. The computer-implemented method of claim 9, further comprising:
registering the patient's pelvis during a surgical procedure;
utilizing the transformation matrix to anchor the holograms of the trajectories of the plurality of bone screws relative to the patient's pelvis.

11. The computer-implemented method of claim 10, wherein the registration device includes a tracking element and the registering the patient's pelvis includes:
docking the registration device with the tracking element to the patient's pelvis; and
recognizing the tracking element of the registration device.

12. The computer-implemented method of claim 8, further comprising:
determining a length of at least one of the plurality of bone screws, wherein the length is determined to prevent the at least one of the plurality of bone screws from extending beyond the patient's pelvis;
generating one or more additional files for presenting additional holograms of the length of the at least one of the plurality of bone screws;
exporting the one or more additional files to the MR-HMD for presenting the additional holograms of the length of the at least one of the plurality of bone screws by the MR-HMD.

13. The computer-implemented method of claim 12, wherein the implant is an acetabular cup with two posterior screw holes and at least one additional screw hole and the determining the trajectories includes:
planning a position and an orientation of the acetabular cup within the acetabulum; and
with the acetabular cup at the planned position and orientation and with approximately 45 millimeter (mm) length bone screws placed at the two posterior screw holes and at the at least one additional screw hole, rotating the acetabular cup until bone screws at the two posterior screw holes are fully within the patient's pelvis;
wherein the determining the length of the at least one of the plurality of bone screws includes reducing the length of the bone screw at the at least one additional screw hole until it is fully within the patient's pelvis.
